(19) United States
(12) Patent Application Publication
(10) Pub. No.: US 2023/0265432 A1
(43) Pub. Date: Aug. 24, 2023

(54) METHODS AND COMPOSITIONS FOR TREATING ACUTE MYELOID LEUKEMIA

(71) Applicant: Sanford Burnham Prebys Medical Discovery Institute, La Jolla, CA (US)

(72) Inventors: Ze'ev A. RONAI, Fallbrook, CA (US); Ali KHATEB, San Diego, CA (US)

(21) Appl. No.: 18/048,382

(22) Filed: Oct. 20, 2022

Related U.S. Application Data
(60) Provisional application No. 63/270,461, filed on Oct. 21, 2021.

(51) Int. Cl. C12N 15/113; A61P 35/02; A61K 31/427; A61K 31/167; A61K 31/506; A61K 38/15; A61K 31/365; A61K 31/7072; A61K 31/4178; A61K 31/519; A61K 31/69; C12Q 1/6886; C12Q 1/6851; G01N 33/574

(52) U.S. Cl. CPC C12N 15/1135; A61P 35/02; A61K 31/427; A61K 31/167; A61K 31/506; A61K 38/15; A61K 31/365; A61K 31/7072; A61K 31/4178; A61K 31/519; A61K 31/69; C12Q 1/6886; C12Q 1/6851; G01N 33/57426; C12N 2310/531; C12Q 2600/158; C12Q 2600/106; G01N 2440/36

(57) ABSTRACT

The present disclosure provides compositions and methods for treating acute myeloid leukemia (AML) using a histone deacetylase (HDAC) inhibitor alone or incombination with a RING finger protein 5 (RNF5) inhibitor and/or a retinoblastoma binding protein 4 (RBBP4) inhibitor. Moreover, RNF5 and/or RBBP4 expression or protein levels in a patient can be measured and used to inform individualized treatment options and dosing regiments. For example, AML patients with lower levels of either RNF5 or RBBP4 may be stratified and treated with one or more HDAC inhibitors leading to improved therapeutic results.

### CROSS-REFERENCE

This application claims benefit of U.S. Provisional Patent Application No. 63/270,461, filed Oct. 21, 2021, which application is incorporated herein by reference in its entirety.

### STATEMENT AS TO FEDERALLY SPONSORED RESEARCH

This invention was made with government support under R35 CA197465 awarded by the National Institutes of Health. The government has certain rights in the invention.

### INCORPORATION BY REFERENCE OF SEQUENCE LISTING

The instant application contains a Sequence Listing which has been submitted electronically in XML format and is hereby incorporated by reference in its entirety. Said XML copy, created on May 12, 2023, is named 42256-797 201 SL.xml and is 36,274 bytes in size.

### BACKGROUND

Acute myeloid leukemia (AML) is a heterogeneous hematological cancer characterized by the accumulation of somatic mutations in immature myeloid progenitor cells. It remains incurable, largely due to its resistance to conventional chemotherapy treatments. Approximately one third of AML patients fail to achieve complete remission in response to chemotherapy, and 40-70% of those who do enter remission relapse within 5 years. Thus, there is an urgent need for more effective chemotherapies to treat AML.

### SUMMARY

Recognized herein is a need for novel pharmaceutical compositions and methods for treating acute myeloid leukemia (AML). The preferred pharmaceutical compositions for treating AML in a subject in need thereof, comprising administering to the subject a therapeutically effective amount of a pharmaceutical composition comprising a really interesting new gene (RING) finger protein 5 (RNF5) inhibitor, or a retinoblastoma binding protein 4 (RBBP4) inhibitor, or both. In some embodiments, the RNF5 inhibitor or the RBBP4 inhibitor comprises a short hairpin ribonucleic acid (RNA), a single guide RNA (sgRNA), or a small molecule. In some embodiments, RBBP4 inhibitor and the RNF5 inhibitor are in different pharmaceutical compositions. In some embodiments, the RBBP4 and the RNF5 inhibitor are administered at different times. In some embodiments, the pharmaceutical composition further comprises a histone deacetylase (HDAC) inhibitor. In some embodiments, the HDAC inhibitor is selected from the group consisting of TMP269, pimelic diphenylamide 106, mocetinostat, romidepsin, and N-acetyldinaline [CI-994]. In some embodiments, the pharmaceutical composition further comprises a compound that increases endoplasmic reticulum (ER) stress. In some embodiments, the compound is thapsigargin or tunicamycin. In some embodiments, the pharmaceutical composition comprises an inhibitor of endoplasmic reticulum associated protein degradation (ERAD). In some embodiments, the inhibitor of ERAD comprises Eeyarestatin I.

In some embodiments, the pharmaceutical composition further comprises an inhibitor of unfolded protein response (UPR). In some embodiments, the inhibitor of UPR comprises GSK2606414. In some embodiments, the pharmaceutical composition further comprises a proteasomal inhibitor. In some embodiments, the proteasomal inhibitor comprises bortezomib. In some embodiments, the method of treating AML further comprises measuring a biomarker in a biological sample obtained from the subject prior to administering to the individual the therapeutically effective amount of the pharmaceutical composition, wherein the measuring the biomarker comprises assaying mRNA expression level and/or protein level of RNF5, RBBP4, or ubiquitinated RBBP4.

In another aspect, provided here in is a method of treating acute myeloid leukemia (AML) in a subject comprising assaying an expression level or an amount of a biomarker in a biological sample obtained from the subject, administering to the subject a therapeutically effective amount of a first pharmaceutical composition when the expression level or the amount of the biomarker is higher than a first predetermined value, and administering to the subject a therapeutically effective amount of a second pharmaceutical composition when the expression level or the amount of the biomarker is lower than a second predetermined value; wherein the second pharmaceutical composition is different from the first pharmaceutical composition. In some embodiments, the biomarker comprises RNF5, RBBP4, or ubiquitinated RBBP4. In some embodiments, the first pharmaceutical composition comprises a RNF5 inhibitor, a RBBP4 inhibitor, a HDAC inhibitor, a UPR inhibitor, a proteasomal inhibitor, an ERAD inhibitor, or any combination thereof. In some embodiments, the first predetermined value is a threshold on an average value in a cohort of AML patients. In some embodiments, the therapeutically effective amount of the first pharmaceutical composition is proportional to the expression level or the amount of the biomarker measured in the subject.

Additional details on the composition and methods described herein can be found in the Detailed Description section of the current application, and in the published paper included in the current application, including its supplemental figures and tables. See Khateb, A., Deshpande, A., Feng, Y. et al. The ubiquitin ligase RNF5 determines acute myeloid leukemia growth and susceptibility to histone deacetylase inhibitors. Nat Commun 12, 5397 (2021), the content of which is incorporated by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

This patent application contains at least one drawing executed in color. Copies of this patent or patent application with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIGS. 1A-1J show expression of RNF5 in AML cell lines and patient samples. FIG. 1A shows RNF5 expression data obtained from the CCLE RNA-seq datasets. Transcripts per million (TPM) for protein-coding genes were calculated using RSEM software. Data is log 2-transformed, using a pseudo-count of 1 w. Box plot is sorted and colored by the average distribution of RNF5 expression in a lineage. Lineages are composed of a number of cell lines. The highest average distributions are shown at left in red. The line within a box represents the mean. FIG. 1B shows representative western blot analysis of RNF5 in peripheral blood mononuclear cells (PBMCs) from healthy and AML patients (Scripps Health). FIG. 1C shows relative abundance of RNF5 protein in PBMCs from AML patients (n=50) and healthy control subjects (n=6) from the Scripps Health Center. RNF5 abundance in U937 cells served as a reference for quantification (presented as the mean±SEM). P=0.008 by unpaired two-tailed t-test. FIG. 1D shows Kaplan-Meier survival curve analysis of AML patients stratified by top high (n=8) versus low (n=42) RNF5 protein (Scripps Health). P=0.05 by two-sided Mantel-Cox log-rank test. FIG. 1E shows Kaplan-Meier survival curve of AML patients stratified by top high (n=14) versus low (n=150) RNF5 transcript levels (TCGA dataset). P=0.009 by tow-sided Mantel-Cox log-rank test. FIG. 1F-1H show levels of RNF5 protein and actin in PBMCs from healthy controls and AML patients (Rambam Health Campus Center). D, diagnosis; PI, post induction; RLP, relapse; RMS, remission. Quantified data are presented as the mean±SEM. P=0.006 by unpaired two-tailed t-test. FIG. 1G shows relative abundance of RNF5 protein in PBMCs from AML patients (n=18) and healthy controls (n=5) from the Rambam Center. Quantified data are presented as the mean±SEM. P=0.006 by unpaired two-tailed t-test. FIGS. 1I-1J show relative abundance of RNF5 protein in AML patient samples (PBMCs) collected before and after induction treatment (FIG. 1I, n=8) or before and after relapse (FIG. 1J, n=5). Lines connect values for the same patient. P=0.054, P=0.0074 by paired two-tailed t-test. Source data are provided as a Source Data file.

FIGS. 2A-2L show RNF5 requirement for AML cell proliferation and survival. FIG. 2A shows growth assay of MOLM-13 and U937 cells transduced with empty vector (pLKO) or two different shRNF5 constructs. Cell growth was analyzed using CellTiter-Glo assay. FIG. 2B shows cell cycle analysis of MOLM-13 and U937 cell lines 5 days after transduction. FIG. 2C shows western blot analysis of cell cycle regulatory proteins in MOLM-13 and U937 AML cells 5 days after transduction. FIG. 2D shows representative images (left) and quantification (right) of colonies in soft agar from MOLM-13 and U937 assessed after 15 days in culture. FIG. 2E shows western blot analysis of apoptosis-related proteins in indicated lines 5 days after transduction (C.casp.3=cleaved caspase-3). FIG. 2F shows growth assay of U937 cells expressing doxycycline-inducible Flag-tagged RNF5 WT, RM or empty vector (EV). Cells were induced 48 h with doxycycline (1 μg/ml) and then transduced with either empty vector (pLKO) or shRNF5 for 5 days. FIG. 2G shows western blot analysis of indicated proteins in U937 transduced as described in FIG. 2F. FIG. 2H shows growth assay of MOLM-13 cells stably expressing Cas9 and transduced with control Renilla-targeting sgRNA or two RNF5-targeting sgRNAs. CRIPSR was performed based on CRIPSR knockout cell pool. FIG. 2I shows western blot analysis of indicated proteins in MOLM-13 cells described in FIG. 2H. FIG. 2J shows growth assay of PDX AML-669 cells after transduction with empty vector (pLKO) or two independent shRNF5 constructs. Quantified data are presented as the mean±SD of two independent experiment. FIGS. 2K-2L show RT-qPCR (FIG. 2K) and western blot (FIG. 2I) analyses confirming RNF5 KD in PDX AML-669 transduced as described in FIG. 2J. Quantified data are presented as the mean±SD (FIGS. 2A, 2F, and 2H) or SEM (FIGS. 2B and 2D) of n=5 (FIG. 2A (left)), n=6 (FIG. 2A (right), FIG. 2B (left)), or n=4 (FIG. 2B (right), FIGS. 2D, 2F, and 211) independent experiments. Western blot data are representative of three experiments. P values were determined using two-way ANOVA followed by Tukey's multiple comparison test (FIGS. 2A, 2F, and 2H) or paired two-tailed t-test (FIGS. 2B and 2D). Source data are provided as a Source Data file.

FIGS. 3A-3G show sensitization of AML cells to ER stress induced apoptosis upon inhibition of RNF5. FIGS. 3A-3B show western blot analysis of indicated proteins in MOLM-13 cells expressing empty vector (pLKO) or shRNF5 #1 and treated with thapsigargin (TG, 1 μM) (FIG. 3A) or tunicamycin (TM, 2 μg/ml) (FIG. 3B) for indicated times. FIG. 3C shows luminescence-based viability assay of MOLM-13 cells expressing inducible shRNF5 and treated with or without doxycycline (DOX, 1 μg/ml) for 3 days before treatment with TG (100 nM) for indicated times. FIG. 3D shows RT-qPCR analysis of CHOP and ATF3 mRNA in MOLM-13 cells expressing pLKO or shRNF5 #1 and treated with TG (100 nM) for indicated times. FIG. 3E shows western blot analysis of cleaved caspase-3 (C. casp.3) and PARP in MOLM-13 cells expressing pLKO or shRNF5 #1 and treated with bortezomib (BTZ, 5 nM) for indicated times. FIG. 3F shows fluorescence-based viability assay of HL-60 cells expressing pLKO or shRNF5 and treated with BTZ (5 nM) for indicated times. Cell viability was determined by flow cytometry of cells stained with annexin V conjugated to fluorescein isothiocyanate and propidium iodide. FIG. 3G shows luminescence viability assay of HL-60 cells expressing pLKO or shRNF5 and treated 48 h with indicated concentrations of BTZ. Data are presented as the mean±SD (FIGS. 3C, 3F, and 3G) or SEM (FIG. 3D) of n=3 (FIGS. 3C and 3G), n=5 (FIG. 3D (left)), or n=4 (FIG. 3D (right) and FIG. 3F) independent experiments. P values were determined using paired two-tailed t-test (FIGS. 3C, 3D, and 3F) or two-way ANOVA (FIG. 3G). ns: not significant. Source data are provided as a Source Data file.

FIGS. 4A-4H show impaired leukemia establishment and progression in vivo upon RNF5 suppression. FIG. 4A shows graph depicting growth in mice of luciferase-expressing U937-pGFL cells transduced with empty vector (pLKO) or inducible shRNF5. Bioluminescence was quantified to monitor disease burden. Data are presented as the mean±SD of 7 mice per group. P values were determined using unpaired two-tailed t-test. FIG. 4B shows Kaplan-Meier survival curves of mice injected with U937-pGFL cells expressing pLKO (n=7 mice/group) or inducible shRNF5 (n=7 mice/group). P<0.001 by two-sided Mantel-Cox log-rank test. FIG. 4C shows schematic representation of the experiment. Lin⁻Sca1⁺-Kit⁺ (LSK) cells were purified from bone marrow of WT or Rnf5^{-/-} mice, transduced in vitro with a GFP-tagged MLL-AF9 fusion gene, and then either analyzed by colony-forming assays in vitro or intravenously injected into sub-lethally irradiated WT C57BL/6 mice. FIG. 4D shows quantification of total colonies (left) or blast-like and differentiated colonies (right) of GFP-MLL-AF9-transformed WT or Rnf5^{-/-} cells after 7, 14, or 21 days in culture. Data are presented as the mean±SD of three independent experiments. P values were determined using paired two-tailed t-test. FIG. 4E shows representative pictures of colonies of GFP-MLL-AF9-transformed WT or Rnf5^{-/-} cells after 7 days in culture. Scale bar 200 μm. FIG. 4F shows Wright-Giemsa-staining of GFP-MLL-AF9-transformed WT or Rnf5^{-/-} cells after 7 days in culture. Scale bar 25 μm. FIG. 4G shows flow cytometric quantification of GFP+ cells in peripheral blood of mice intravenously injected with GFP-MLL-AF9-transformed WT (n=4) or Rnf5^{-/-} (n=4) cells at days 15 and 28 post-injection. Data are presented as the mean±SD. P=0.002 by unpaired two-tailed t-test. Gating strategy is provided in FIG. 15B. FIG. 4H shows Kaplan-Meier survival curves of mice injected with GFP-MLL-AF9-transformed WT or Rnf5^{-/-} cells. Data are from two independent experiments (n=4 mice/group per experiment). P<0.001 by two-sided Mantel-Cox log-rank test. Source data are provided as a Source Data file.

FIGS. 5A-5N show transcriptional and survival analysis of RNF5 or RBBP4 deficient AML cells. FIG. 5A shows Venn diagram analysis of RNA-seq results showing upregulated (red) and downregulated (green) genes in AML lines following RNF5 KD. Overlapping areas indicate commonly modulated genes. FIG. 5B shows Heatmap of RNA-seq data performed on pLKO or shRNF5 AML lines, as indicated beneath maps (see Methods). FIG. 5C shows RT-qPCR validation of genes deregulated by RNF5-KD. Data are presented as the mean±SD of n=5 or 6 (ANXA1) or n=3 (NCF1 and CDKN1A) independent experiments. FIG. 5D shows top ten drug screening results from the LINCS matched with transcriptomic data from shRNF5 MOLM-13 line. Values are overall z-scores from IPA Analysis Match database. HDAC1 inhibitor results are shown in red. FIG. 5E shows shRNF5 transduction of MOLM-13 or HL-60 promotes changes seen in NPC, HEPG2, A549 and PC3 cancer cell lines treated with the HDAC1 inhibitor mocetinostat. Z-score was calculated by IPA: A positive z-score predicts pathway activation; a negative z-score predicts inhibition. FIG. 5F shows results of LC-MS/MS analysis present log₂-transformed ratio of proteins in anti-Flag immunoprecipitates of RNF5-overexpressing versus control cells. Green, proteins significantly enriched in RNF5-overexpressing cells; blue, enriched proteins in the ERAD pathway. RNF5 and RBBP4 are indicated in red. FIG. 5G shows co-expression of RBBP4 and indicated RNF5 target genes in AML samples analyzed in cBioPortal using the TCGA database (Pearson correlation, P<0.0001, n=165). FIG. 5H shows WB of RBBP4 in PBMCs from healthy subjects and AML patients (Scripps Health). FIG. 5I shows overall survival rate (performed using GEPIA and TCGA) of AML patients expressing high (30%) or low (70%) levels of RBBP4 transcripts. TPM: Transcripts Per Million. HR: hazard ratio. FIG. 5J shows growth assay of MOLM-13 cells after transduction with indicated constructs. Data are presented as the mean±SD of n=3 independent experiments. FIG. 5K shows WB analysis of indicated proteins in MOLM-13 cells expressing indicated constructs. FIG. 5L shows RT-qPCR analysis of genes deregulated by RNF5-KD in MOLM-13 cells expressing the indicated constructs. Data are presented as the mean±SD of n=5 (RBBP4), n=4 (NCF1 and CDK1V1A), or n=3 (ANXA1) independent experiments. FIG. 5M shows bioluminescent images of representative mice 4 weeks following transplantation of U937-pGFL expressing the indicated constructs. FIG. 5N shows Kaplan-Meier survival curves of mice injected with U937-pGFL cells expressing indicated vectors. P=0.02 and P=0.001 by two-sided Mantel-Cox log-rank test. P values were determined using paired two-tailed t-test (FIGS. 5C and 5L) or two-way ANOVA followed by Tukey's multiple comparison test FIG. 5J. Source data are provided as a Source Data file.

FIGS. 6A-6N show ubiquitination of RBBP4 by RNF5 and regulation of RNF5 target genes. FIG. 6A shows schematic showing full-length and mutants forms of RNF5. FIG. 6B shows immunoprecipitation (IP) and Western blot (WB) analysis of HEK293T cells transfected with Flag-tagged forms of full-length RNF5 (WT), the catalytically inactive RING domain mutant (RM), or the C-terminal transmembrane domain deletion mutant (ACT). Cells were treated with MG132 (10 μm, 4 h) before lysis. FIG. 6C shows IP and WB of HEK293T cells co-expressing Myc-RBBP4 and Flag-tagged RNF5-WT treated with MG132 (10 μm, 4 h) before lysis. FIG. 6D shows IP and WB of ectopically expressed doxycycline-inducible Flag-tagged RNF5 and endogenous RBBP4 in MOLM-13 cells. Cells were incubated 2 days with or without doxycycline (1 μg/ml) and with MG132 (10 μm, 4 h) before lysis. FIG. 6E shows WB of anti-Myc IP from lysates of HEK293T cells co-expressing Myc-RBBP4, hemagglutinin-tagged ubiquitin (HA-Ub), and indicated Flag-tagged RNF5 constructs. Cells were treated with MG132 (10 μm, 4 h) before lysis. FIG. 6F shows WB of indicated proteins in MOLM-13 cells expressing empty vector (pLKO) or indicated shRNF5 construct. FIG. 6G shows WB analysis of indicated proteins in MOLM-13 cells expressing empty vector or doxycycline-inducible Flag-tagged RNF5. FIG. 6H shows WB of anti-Myc IP and lysates of HEK293T cells co-expressing Myc-RBBP4, Flag-tagged RNF5, and different HA-tagged ubiquitin mutants (K29, K11, K6, K27 and K33). MG132 (10 μm, 4 h) was added before lysis. FIG. 6I shows IP and WB for the interaction of RBBP4 with HDAC1, HDAC2, or EZH2 in MOLM-13 cells expressing indicated constructs. FIG. 6J shows IP and WB for RBBP4 interaction with HDAC1, HDAC2, or EZH2 in MOLM-13 cells expressing indicated constructs. MG132 (10 μm, 4 h) was added before lysis. FIG. 6K shows ChIP and qPCR reveal the enrichment of RBBP4 (normalized to input) at indicated gene promoters in MOLM-13 cells expressing indicated constructs. FIGS. 6L-6N show ChIP and qPCR reveal the enrichment of H3K9ac, H3K27ac, or H3K27me3 (normalized to input) at indicated gene promoters in MOLM-13 cells expressing indicated constructs. Data in FIG. 6K and FIG. 6L are presented as mean±SEM of n=4 (ANXA1) or n=3 (NCF1 and CDK1V1A) independent experiment. Data in FIG. 6M and FIG. 6N are mean of n=2 independent experiments. The P values were determined using paired two-tailed t-test (FIGS. 6K and 6L). Source data are provided as a Source Data file.

FIGS. 7A-7N show sensitization of AML cells to HDAC inhibitors by RNF5 inhibition. FIG. 7A shows schematic showing experimental design of the epigenetic screen. FIG. 7B shows Log₂-transformed ratios of the relative viability of doxycycline-induced (+Dox) versus uninduced (−Dox) U937 cells treated with compounds for 6 days. Red dots represent compounds that altered viability of RNF5-KD more than of uninduced cells, blue dots represent candidate HDAC inhibitors, and grey dots represent the remaining compounds tested. FIG. 7C shows viability of U937 cells expressing indicated constructs after treatment for 24 h with CI-994. FIG. 7D shows U937 cell viability after treatment with 3.5 nM FK228. FIG. 7E shows viability of U937 cells or MOLM-13 cells expressing indicated constructs 24 h following FK228 treatment. FIG. 7F shows WB of apoptotic markers in MOLM-13 and U937 cells expressing indicated constructs and incubated with or without FK228 (4 nM for 24 h). FIG. 7G shows viability of U937 cells expressing indicated constructs and treated for 24 h with FK228. EV-pLKO, control cells; EV-shRNF5, cells expressing empty vector and shRNF5; RNF5-pLKO, cells overexpressing RNF5 and pLKO vector; RNF5-shRNF5, cells overexpressing RNF5 and shRNF5. FIG. 7H shows viability of MOLM-13 cells expressing indicated constructs 24 h following FK228 treatment. FIG. 7I shows ChIP and qPCR indicating H3K9ac enrichment (normalized to input) at indicated gene promoters in MOLM-13 cells expressing indicated constructs. Data are presented as the mean±SD of two independent experiments. FIG. 7J shows RT-qPCR analysis of indicated genes in MOLM-13 cells expressing indicated constructs following FK228 treatment (4 nM, 15 h). FIG. 7K shows viability assay of 4 primary AML blasts (Scripps Health) 48 h following FK228 treatment. FIG. 7L shows WB analysis of RNF5 and RBBP4 in AML patient samples used for the ex-vivo drug analysis in FIG. 7L. FIG. 7M shows WB quantification of RNF5 protein levels in FIG. 7L normalized to actin. FIG. 7N shows Kaplan-Meier plot showing survival analysis of AML patients segregated based on a median synthetic lethality (SL) score. Co-occurrence of low HDAC and RNF5 transcript levels in a patient's tumor (high SL score; blue line), compared with the rest of the patients (low score, yellow line). Data are presented as the mean±SD of n=4 (FIGS. 7C and 7D), n=3 (FIGS. 7E, 7G, and 7J), or n=5 FIG. 7H experiments. P values were determined using paired two-tailed t-test (FIGS. 7D and 7J) or two-way ANOVA followed by Tukey's multiple comparison test (FIGS. 7C, 7E, 7G, and 7H). Source data are provided as a Source Data file.

FIGS. 8A-8J show inverse correlation between RNF5 protein and transcript levels and AML patient outcome. FIG. 8A shows CCLE data copy number analysis of the RNF5 locus across cancer cell lines from various tissue sources 1. Line within the box blot show the mean log 2 copy number for each tissue. FIG. 8B shows western blot (WB) analysis of RNF5 in lysates made from indicated cancer cell lines: AML, acute myeloid leukemia; CML, chronic myeloid leukemia; ALL, acute lymphoblastic leukemia; CLL, chronic lymphoblastic leukemia; MM, multiple myeloma; MCL, mantle cell lymphoma; and melanoma. FIGS. 8C-8D show abundance of RNF5 and histone H3 in PBMCs and CD34+ from healthy control (Healthy B and CD34+) subjects and AML patients from the Scripps Health Center. FIG. 8E shows RT-qPCR analysis of RNF5 mRNA in PBMCs from healthy control subjects (n=4) and AML patients (n=17) from the Scripps Health Center. Data are presented as the mean±SEM. P=0.829 by two-tailed unpaired t-test. FIG. 8F shows blast count percentages in AML samples expressing high (n=8) versus low (n=35) RNF5 protein. The horizontal band inside boxes indicates the median, the bottom and top edges of the box 25th-75th percentiles and the whiskers indicate the min to max. P=0.254 by two-tailed unpaired t-test. FIG. 8G shows Pearson correlation analysis between percentage of blasts and RNF5 protein levels in AML samples (n=43) from Scripps Health. P=0.586 by two-tailed Pearson Coefficient. FIG. 8H shows relative RNF5 protein levels in AML samples (Scripps Health Center) positive or negative for NPM1. Data are presented as the mean±SEM. P=0.082 (left) P=0.515 (right) by two-tailed unpaired t-test. FIG. 8I shows relative RNF5 protein levels in AML samples (Rambam Health) positive or negative for NPM1 or FLT3 mutations. Data are presented as the mean±SEM. P=0.926 (left) P=0.296 (right) by two-tailed unpaired t-test. FIG. 8J shows WB analysis of RNF5 protein in PBMCs from healthy donors or AML patients in the Rambam Center cohort. Arrow indicate RNF5 position. The upper band is unspecific.

FIGS. 9A-9H show RNF5 requirement for AML cell growth. FIG. 9A shows luminescence-based growth assay of U937 cells expressing empty vector (pLKO) or shRNF5 #3. FIG. 9B shows growth assay of Jurkat cells expressing pLKO or two different shRNF5 constructs. FIG. 9C shows growth assay of K562 cells expressing pLKO or three different shRNF5 constructs. Western blots below B and C show knockdown efficiency. FIG. 9D shows western blot analysis of MOLM-13 and U937 cells 5 days after transduction with pLKO or shRNF5 #3. Data are representative of three experiments. FIGS. 9E-9F show luminescence-based growth assays of HL-60 or THP-1 cells transduced with pLKO or shRNF5 #1. FIG. 9G shows plate images (left) and quantification (right) of HL-60 colonies in soft agar. Colonies were assessed after 14 days in culture. FIG. 9H shows western blot analysis of the indicated proteins in HL-60 and THP-1 cells 5 days after transduction with pLKO, shRNF5 #1, or shRNF5 #2. Data are representative of three experiments. Quantified data are presented as the mean±SD and are representative of n=3 (FIGS. 9A-9C) or n=4 (FIGS. 9E-9G) independent experiments. P values were determined using two-tailed paired t-test.

FIGS. 10A-10B show sensitization of AML cells to ER stress induced apoptosis by RNF5 KD. FIG. 10A shows luminescence growth assay of HL-60 cells expressing pLKO or shRNF5 after treatment with tunicamycin (2 μg/mL) for indicated times. Data are presented as mean±SEM of n=6 independent experiments. FIG. 10B shows RT-qPCR analysis of UPR-related genes in HL-60 cells treated with thapsigargin (1 μM) for indicated times. Data are presented as mean±SEM of n=5 (CHOP and sXBP1) or n=4 (ATF3) independent experiments. P values were determined using two-tailed paired t-test. Ns: not significant.

FIGS. 11A-11E show antagonization of leukemia establishment and progression in vivo by RNF5 suppression. FIG. 11A shows U937-pGFL cells expressing pLKO or inducible shRNF5 were treated 3 days with Dox (1 μg/mL) and then subjected to Western analysis to detect RNF5. GADPH served as a loading control. FIG. 11B shows bioluminescent images of representative mice injected with U937-pGFL expressing empty vector (pLKO) or inducible shRNF5 at days 18, 25 and 32. FIG. 11C shows RT-qPCR validation of RNF5 KD from splenocytes of mice injected with pLKO (n=3) or shRNF5 (n=3) cells. Data are presented as mean±SEM. FIG. 11D shows western blot analysis of p27 in lysates of splenocytes from mice injected with empty vector (pLKO) or shRNF5 cells. Ponceau staining served as loading control. FIG. 11E shows western blot analysis of RNF5 in lysates from WT and Rnf5−/−MLL-AF9 transformed cells. H3 served as loading control.

FIGS. 12A-12P show transcription modulation in AML cells by RNF5 activity. FIG. 12A shows top canonical pathways identified by Ingenuity Pathway Analysis comparing genes differentially expressed in indicated AML cell lines upon RNF5-KD. FIG. 12B shows RT-qPCR analysis of a select subset of genes identified as deregulated upon RNF5-KD by RNA-seq analysis. FIG. 12C shows top ten drug screening results from LINCS database matched with transcriptomic changes in shRNF5 HL-60 line. Values are overall z-scores from IPA Analysis Match database. HDAC1 inhibitor results are shown in red. FIG. 12D shows RNF5 interaction network generated from immunoprecipitation data and Cytoscape. Colors correspond to indicated pathways. FIG. 12E shows pathway enrichment analysis displaying gene counts (log 2 transformed) and the corresponding false discovery rate (−log 10 transformed) for each pathway. FIGS. 12F-12I show co-expression of RBBP4 (FIG. 12F), EZH2 (FIG. 12G), HDAC1 (FIG. 12H) or HDAC2 (FIG. 12I) mRNA and the indicated RNF5 target genes in AML analyzed in cBioPortal using data from TCGA. Pearson correlation, P<0.0001, n=165. FIG. 12J shows analysis of RBBP4 expression in different human cancers from the cBioPortal using data from TCGA. FIG. 12K shows western blot analysis of RBBP4 in PBMCs from healthy control subjects and AML patients from Scripps Health and Rambam Medical Centers cohorts. RMS, remission. FIG. 12L shows growth assay of U937 cells after transduction with empty vector (pLKO) or the indicated shRBBP4 constructs. Data are presented as the mean±SD of two independent experiments. FIG. 12M shows western blot analysis of indicated proteins in U937 cells expressing empty vector (pLKO) or two different shRBBP4 constructs. FIG. 12N shows western blot confirmation of RBBP4 KD in U937-pGFL cells used for the xenograft experiment. FIG. 12O shows RT-qPCR confirmation of RBBP4 KD in U937-pGFL cells used for the xenograft experiment. FIG. 12P shows western blot analysis of RBBP4 in lysates of splenocytes from mice injected with empty vector (pLKO) or shRBBP4 cells. Quantified data are presented as the mean±SD and are representative of at least three independent experiments unless stated otherwise. P values were determined using two-tailed paired t-test.

FIGS. 13A-13I show interaction and ubiquitination of RBBP4 by RNF5. FIG. 13A shows growth assay of K-562 cells following transduction with empty vector (pLKO) or the indicated shRBBP4 constructs. Western blot shows knockdown efficiency. Data are presented as the mean±SD of n=3 independent experiment. FIG. 13B shows growth assay of Jurkat cells after transduction with pLKO or the indicated shRBBP4 constructs. Western blot shows knockdown efficiency. Data are presented as the mean±SD of n=2 independent experiment. FIG. 13C shows western blot analysis of anti-Myc immunoprecipitates and lysates of HEK293T cells co-expressing Myc-RBBP4, HA-Ub, and shRNF5. Cells were treated with MG132 (10 μm) for 4 h before lysis. FIG. 13D shows western blot analysis of anti-RBBP4 immunoprecipitates and lysates of MOLM-13 cells expressing the indicated shRNF5 constructs. Cells were treated with MG132 (10 μm) for 4 h before lysis. Quantification of the ubiquitination smear relative to the amount of RBBP4 pull down is shown at the top. FIG. 13E shows western blot analysis of anti-Myc immunoprecipitates and lysates of HEK293T cells co-expressing Myc-RBBP4, HA-Ub, and the indicated Flag-tagged RNF5 constructs. Cells were treated with MG132 (10 μm) 4 h before lysis. FIG. 13F shows western blot analysis of indicated proteins in HEK293T cells transfected with Myc-RBBP4 and Flag-RNF5. FIG. 13G shows western blot analysis of RBBP4 and RNF5 in indicated fractions of MOLM-13 cells expressing pLKO or shRNF5 #1. CE, cytoplasmic extract; ME, membrane extract; NE, nuclear extract; CB, chromatin bound. Histone H3, HSP90, and calreticulin serve as controls for chromatin, cytosol, and membrane fractions, respectively. FIG. 13H shows immunofluorescence staining of RBBP4 (red) in control or shRNF5-expressing MOLM-13 cells. Nuclei were stained with DAPI (blue). Scale bar 60 μM. Western blot below shows RNF5-KD efficiency. FIG. 13I shows immunoprecipitation and Western blot analysis of the interaction of RBBP4 with HDAC1, HDAC2, or EZH2 in U937 cells expressing indicated constructs. Cells were treated with MG132 (10 μm) 4 h before lysis.

FIGS. 14A-14K show AML cell sensitization to HDAC inhibition by RNF5KD. FIG. 14A shows viability of HL-60 cells expressing pLKO or two shRNF5 constructs after treatment for 24 h with CI-994. Western blot below confirms RNF5-KD. FIG. 14B shows viability of MOLM-13 cells after treatment for 24 h with 3.5 nM FK228. FIGS. 14C-14D show viability of HL-60 (FIG. 14C) or THP-1(FIG. 14D) cells expressing pLKO or shRNF5 constructs after treatment for 24 h with FK228. Western blot below confirms RNF5-KD. FIG. 14E shows viability of MOLM-13 cells stably expressing Cas9 and transduced with control Renilla-or RNF5-targeting sgRNA and treated for 24 h with FK228. Western blot shows reduction in RNF5 levels. FIG. 14F shows viability of MOLM-13, U937, MV-4-11, THP-1, and HL-60 cells after treatment for 24 h with FK228. FIG. 14G shows viability of MV-4-11 cells expressing pLKO or shRNF5 constructs and treated for 24 h with indicated FK228 concentrations. Data are presented as the mean±SD of n=2 independent experiments. FIG. 14H shows viability of U937 cells expressing pLKO or shRBBP4 and treated for 24 h with the indicated FK228 concentrations. FIG. 14I shows RT-qPCR analysis of LIMK1 mRNA in MOLM-13 cells expressing empty vector (pLKO) or shRNF5 #1 and treated 15 h with 4nMFK228. Data are presented as the mean±SD of n=2 independent experiments. FIG. 14J shows viability of K-562 cells expressing pLKO or three shRNF5 constructs after treatment for 30 h with the indicated FK228 concentrations. Western blot confirms RNF5-KD. FIG. 14K shows viability of Jurkat cells expressing pLKO or two shRNF5 constructs after treatment for 24 h with indicated FK228 concentrations. Western blot confirms RNF5-KD. Quantified data are presented as the mean±SD of n=3 (FIGS. 14A, 14E-H, and 14J) or n=4 (FIGS. 14B-14D) independent experiments. P values were determined using two-tailed t-test (FIG. 14B) or two-way ANOVA (FIG. 14H) followed by Tukey's multiple comparison test (FIGS. 14A, 14C, 14E, and 14F).

FIGS. 15A-15C show gating strategies for FACS analysis experiments. FIG. 15A shows a representative example of gating used for sorting of U937-pGFL and transformed MLL-AF9 GFP+ cells. Related to FIGS. 4A and 4C. FIG. 15B shows gating strategy of GFP+ cells quantification in peripheral blood of mice intravenously injected with GFP-MLL-AF9-transformed cells (FIG. 4G). FIG. 15C shows gating strategy for Annexin-V/PI staining (FIG. 3F).

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The terminology used herein is for the purpose of describing particular cases only and is not intended to be limiting.

As used herein, the singular forms “a”, “an” and “the” are intended to include the plural forms as well, unless the context clearly indicates otherwise.

When a range of values is provided, it is to be understood that each intervening value between the upper and lower limit of that range, and any other stated or intervening value in that stated range is encompassed within the scope of the present disclosure. Where the stated range includes upper or lower limits, ranges excluding either of those included limits are also included in the present disclosure.

As used herein, the term “biomarker” generally refers to an indicator, e.g., predictive, diagnostic, and/or prognostic, which can be detected in a sample. The biomarker may serve as an indicator of a particular subtype of a disease or disorder (e.g., AML) characterized by certain, molecular, pathological, histological, and/or clinical features. In some embodiments, a biomarker is a gene. Biomarkers include, but are not limited to, polynucleotides (e.g., DNA, and/or RNA), polypeptides, polypeptide and polynucleotide modifications (e.g., posttranslational modifications), carbohydrates, and/or glycolipid-based molecular markers.

As used herein, the term “sample” generally refers to a composition that is obtained or derived from a subject and/or individual of interest that contains a cellular and/or other molecular entity that is to be characterized and/or identified, for example based on physical, biochemical, chemical and/or physiological characteristics. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and any combinations thereof.

As used herein, the term “effective amount” of an agent generally refers to an amount effective, at dosages and for periods of time necessary, to achieve the desired therapeutic or prophylactic result. A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects.

As used herein, the terms “treat”, “treating”, or “treatment”, include reducing, alleviating, abating, ameliorating, relieving, or lessening the symptoms associated with a disease, disease sate, or indication (e.g., addiction, such as opioid addiction, or pain) in either a chronic or acute therapeutic scenario. Also, treatment of a disease or disease state described herein includes the disclosure of use of such compound or composition for the treatment of such disease, disease state, or indication.

As used herein, the term “pharmaceutical formulation” or “pharmaceutical composition” generally refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the phrase “based on” generally means that the information about one or more biomarkers is used to inform a diagnosis decision, treatment decision, information provided on a package insert, or marketing/promotional guidance, etc.

As used herein, the term “subject,” generally refers to an individual from whom a biological sample is obtained. The subject may be a mammal or non-mammal. The subject may be human, non-human mammal, animal, ape, monkey, chimpanzee, reptilian, amphibian, avian, or a plant. The subject may be a patient. The subject may be displaying a symptom of a disease. The subject may be asymptomatic. The subject may be undergoing treatment. The subject may not be undergoing treatment. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer, cervical cancer, etc.) or an infectious disease.

The present invention demonstrates, inter alia, that the protein RNF5 plays an unusual and role in AML. Marking aberrant proteins for destruction, RNF5 binds with a second cell protein called RBBP4 to control expression of genes implicated in AML. These findings have important implications for improving AML patient outcomes. For example, if AML patients have low levels of RNF5 and/or RBBP4, they may respond better to treatment with HDAC inhibitors.

Increased Expression of RNF5 in AML Patient Samples Correlates with Poor Prognosis

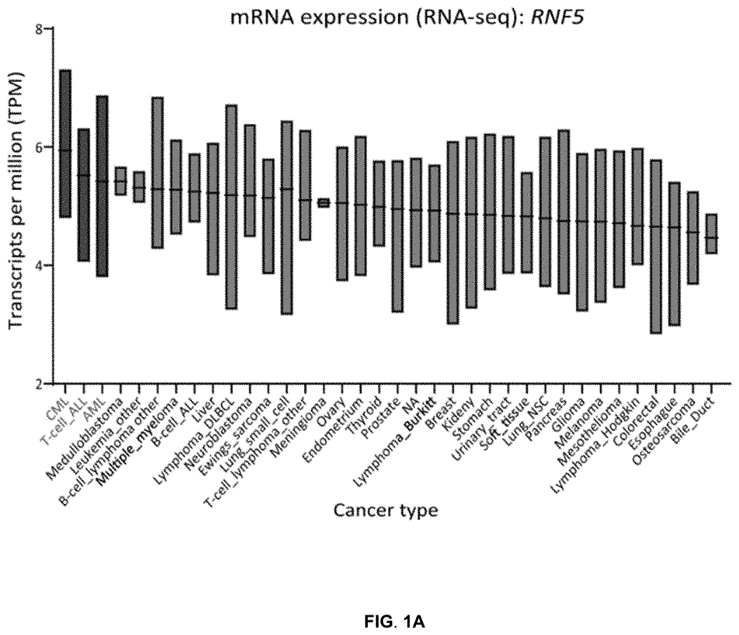

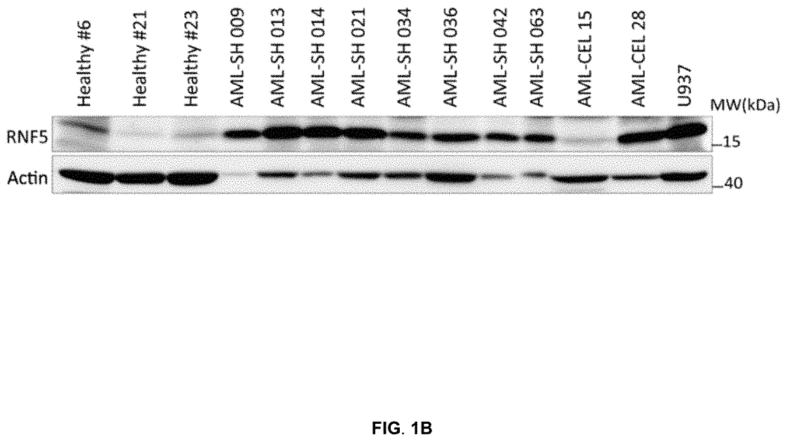

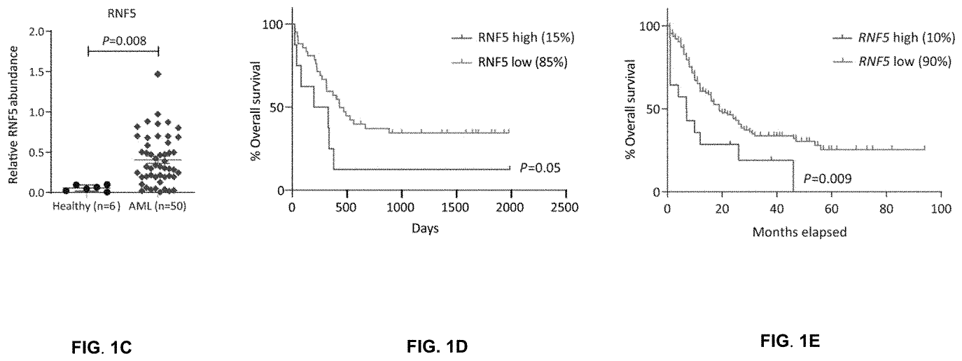

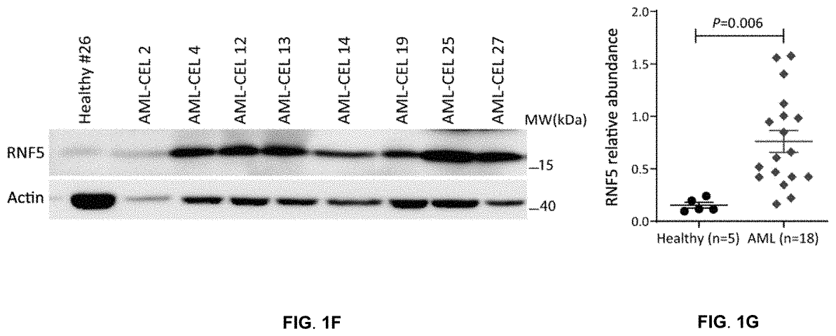

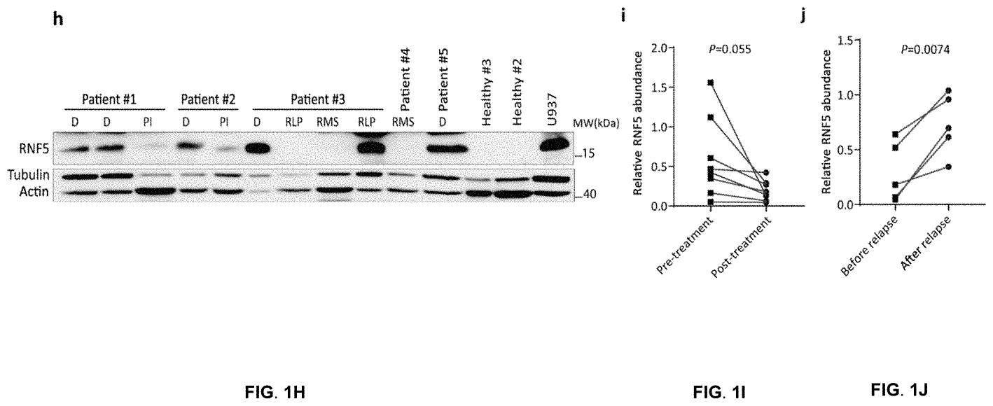

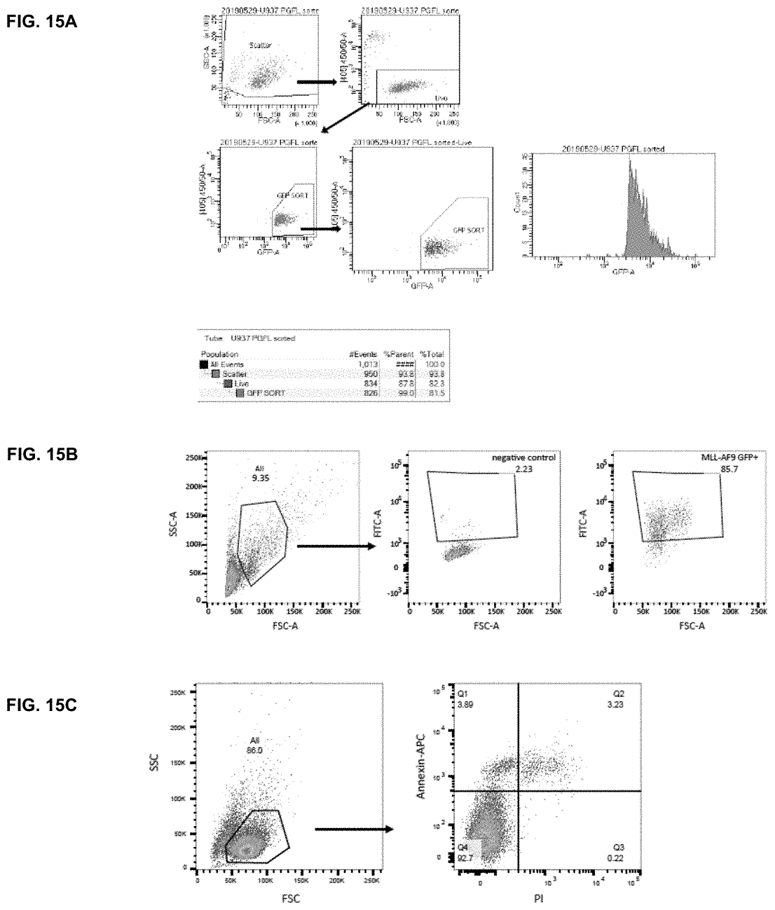

Analysis of RNA-seq datasets for various cancer cells in the Cancer Cell Line Encyclopedia database identified higher copy number and levels of RNF5 transcripts in AML, chronic myeloid leukemia (CIVIL), and T-cell acute lymphoblastic leukemia (T-ALL) relative to other tumor types (FIGS. 1A and 15A). Higher levels of RNF5 protein were confirmed in AML and CIVIL cell lines compared with other cancer lines (FIG. 15). To assess the clinical relevance of RNF5 expression in AML, levels of RNF5 mRNA and protein in peripheral blood mononuclear cells (PBMCs) from independent cohorts of AML patients were analyzed. Similar to results in AML lines, the average abundance of RNF5 protein was significantly higher in PBMCs from AML patients relative to control samples (CD34⁺ and PBMCs) (FIGS. 1B, 1C, 15C, and 15D). Patient cohort included equal number of females (24; median age=57.7) and males (24; median age=63.4; See Table 1). Given that RNF5 is a ubiquitin ligase, its transcript levels are not as reflective of activity as are protein levels, as self-degradation or other post translational modifications can alter RNF5 subcellular localization, availability and/or activity. Indeed, analysis of patient cohorts revealed a significant increase in RNF5 protein but not transcript levels in patients as compared to healthy subjects (FIGS. 1B, 1C, and 15E). Stratification of the 50 patients into two groups based on the top high (N=8, 15%) and low (n=42, 85%) RNF5 protein levels revealed that high abundance coincided with poor overall survival (P=0.05, FIG. 1D). Notably, this difference was not due to blast counts, as they did not differ significantly among patients showing high or low RNF5 levels (FIGS. 15F and 15G). Independent analysis of AML patients (n=154) based on The Cancer Genome Atlas (TCGA) dataset confirmed a significant positive correlation between high RNF5 expression (10%) and poor survival (P=0.009, FIG. 1E). Notably, there was no pattern of RNF5 abundance that either positively or negatively correlated with the presence of FLT3 or NPM1 mutations (FIGS. 1511 and 15I), suggesting that the significance of RNF5 activity to AML, may not depend on any specific oncogenic driver(s) or activation of particular signaling pathways.

TABLE 1
Deidentified patient data

| | |
|---|---|
| Scripps Health Center | Rambam Health Care Campus |

| Pt | Sex | Code | Pt | Sex |
|---|---|---|---|---|
| 2 | F | 90_CEL_24 | 1 | F |
| 4 | F | 123_CEL_24 | 1 | |
| 5 | M | 90_CEL_28 | 2 | M |
| 6 | M | 123_CEL_28 | 2 | |
| 7 | M | 90_CEL_20 | 3 | M |
| 8 | M | 90_CEL_29 | 3 | M |
| 9 | M | 90_CEL_25 | 3 | M |
| 10 | M | 90_CEL_19 | 4 | M |
| 11 | F | 90_CEL_4 | 4 | M |
| 12 | M | 90_CEL_9 | 4 | M |
| 14 | F | 90_CEL_16 | 5 | F |
| 15 | M | 90_CEL_2 | 5 | F |
| 16 | F | 90_CEL_26 | 5 | F |
| 17 | F | 90_CEL_23 | 5 | F |
| 18 | F | 90_CEL_3 | 6 | F |
| 19 | F | 90_CEL_8 | 6 | F |
| 20 | M | 90_CEL_17 | 6 | F |
| 21 | M | 90_CEL_14 | 6 | F |
| 22 | M | 90_CEL_6 | 6 | F |
| 23 | F | 90_CEL_11 | 7 | M |
| 27 | F | 90_CEL_12 | 8 | M |
| 28 | M | 90_CEL_13 | 8 | M |
| 29 | F | 90_CEL_10 | 8 | M |
| 30 | F | 90_CEL_22 | 9 | M |
| 31 | F | 93_CEL_21 | 9 | M |
| 32 | M | 90_CEL_5 | 10 | F |
| 34 | F | 90_CEL_7 | 11 | F |
| 36 | M | 90_CEL_1 | 11 | F |
| 37 | M | 90_CEL_18 | 11 | F |
| 38 | F | 90_CEL_15 | 11 | F |
| 40 | M | 90_CEL_30 | 11 | F |
| 42 | F | 90_CEL_27 | 11 | F |
| 43 | M | | | |
| 44 | M | | | |
| 45 | F | | | |
| 50 | M | | | |
| 51 | F | | | |
| 52 | M | | | |
| 58 | F | | | |
| 60 | M | | | |
| 61 | M | | | |
| 65 | M | | | |
| 67 | M | | | |
| 69 | F | | | |
| 72 | F | | | |

Assessment of an independent AML, patient cohort (from the Rambam Health Campus Center, Haifa, Israel which included multiple samples obtained from 5 females with median age of 59.4 and 6 males with median age of 57.3, as detailed in Table 1) confirmed higher levels of RNF5 protein in AML patient blood samples (n=18) relative to samples taken from healthy donors (n=5) (FIGS. 1F and 1G). Because this cohort included samples taken from patients both prior to and following therapy, RNF5 abundance before and after therapy and at remission or relapse stages were compared. Notably, RNF5 abundance markedly decreased following chemotherapy and during remission (n=8) (FIGS. 1H, 1I, and 15J). Conversely, RNF5 levels at diagnosis were similar to those seen in patients that either relapsed or were refractory to treatment (n=5) (FIGS. 1H and 1J). These results suggest that RNF5 levels in AML blasts may serve as a prognostic marker for AML.

### RNF5 is Required for AML Cell Proliferation and Survival

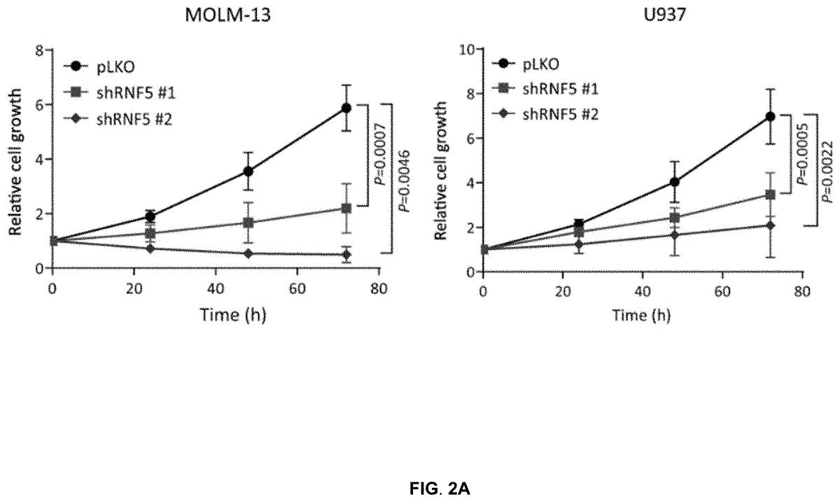

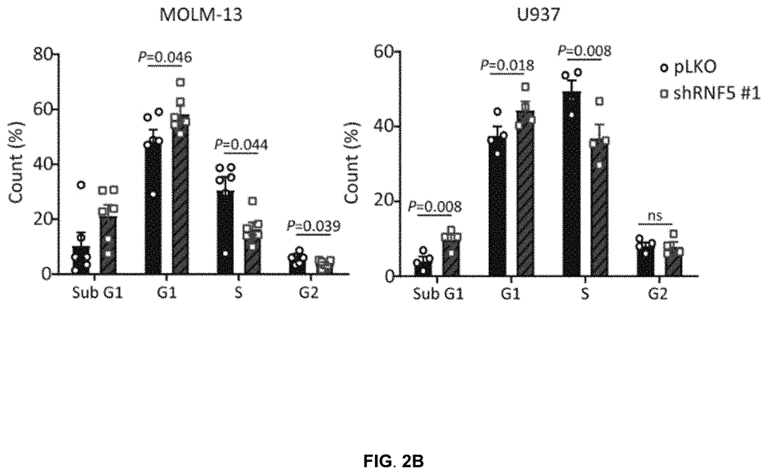

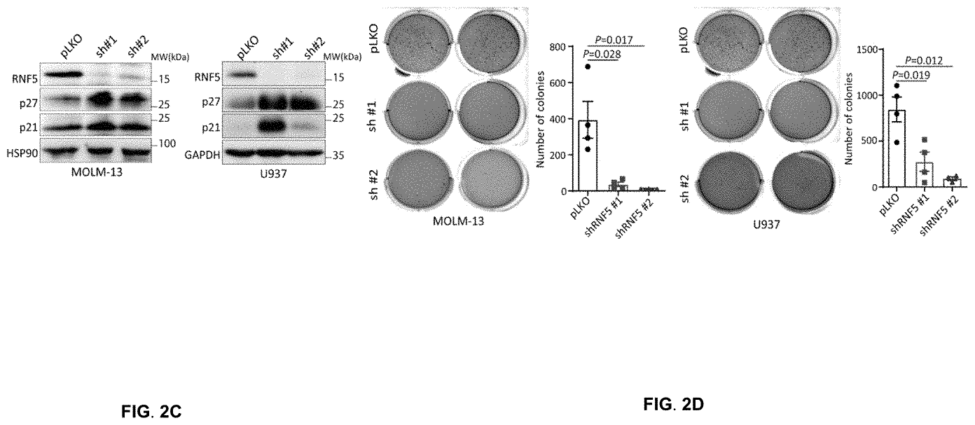

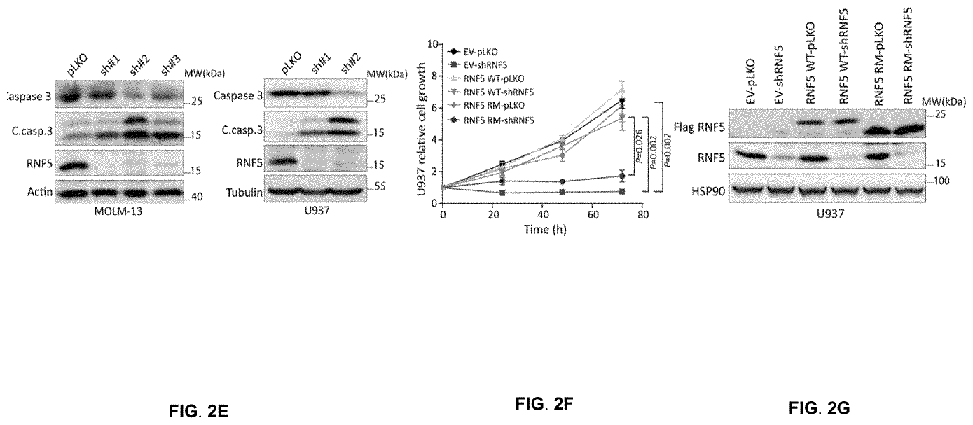

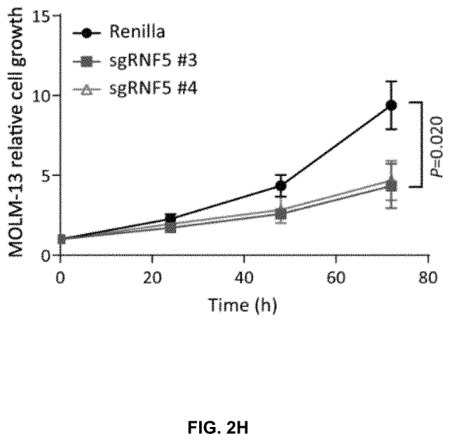

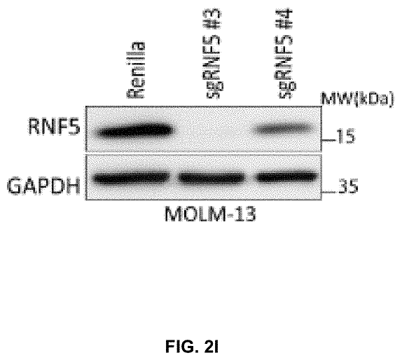

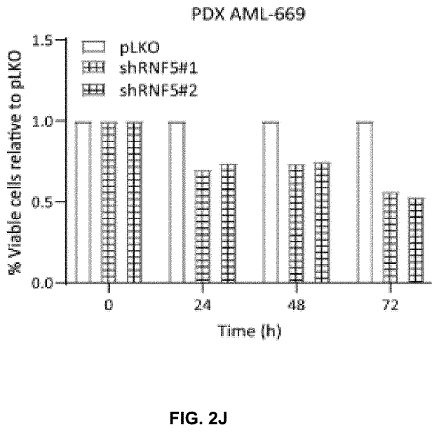

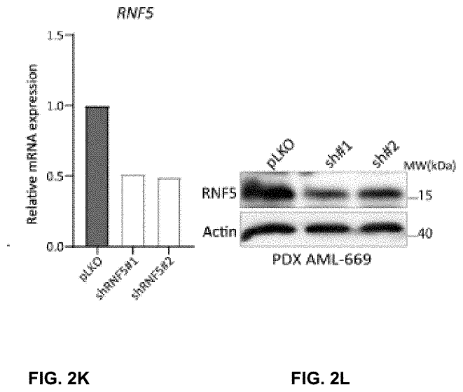

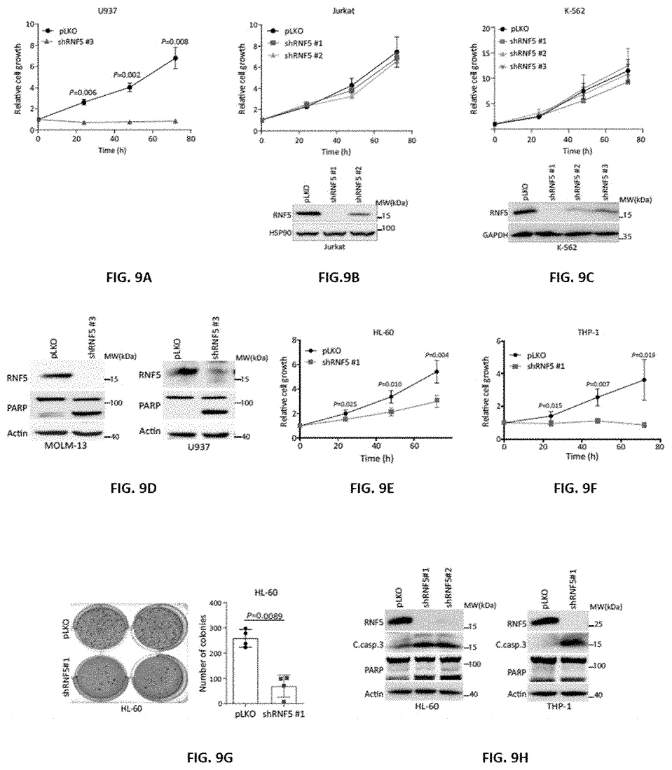

RNF5 knockdown (RNF5-KD) inhibits leukemia cell growth in vitro. Surprisingly, KD using RNF5-targeting short hairpin RNAs (shRNF5) decreased viability and attenuated growth of MOLM-13 and U937 AML lines (FIGS. 2A and 9A) but not of CIVIL (K-562) or T-ALL (Jurkat) lines (FIGS. 9B and 9C). RNF5 KD in MOLM-13 or U937 AML cells also promoted accumulation of cells in the G1 phase of the cell cycle (FIG. 2B), an effect accompanied by increases in levels of the cell cycle regulatory proteins p27 and p21 (FIG. 2C). Moreover, AML cells MOLM-13 and U937 with RNF5-KD showed reduced colony formation in soft agar relative to controls (FIG. 2D) and increased abundance of proteins associated with apoptosis, as reflected by assessing levels of cleaved forms of caspase-3 (FIG. 2E) and poly ADP ribose polymerase (PARP) (FIG. 9D). Effects of RNF5-KD on U937 and MOLM-13 cells were confirmed in two additional AML cell lines HL-60 and THP-1 (FIGS. 9E-9H). Importantly, re-expression of RNF5 WT, but not the catalytically inactive RING mutant (RNF5 RM), restored cell proliferation (FIGS. 2F and 2G), confirming the specificity of these phenotypes and suggesting that RNF5 catalytic activity is required for AML cell proliferation.

To verify changes seen upon KD, CRISPR-Cas9 gene editing technology was used to deplete RNF5 in MOLM-13 cells stably expressing Cas9 using RNF5-targeting guide RNAs (sgRNAs). Relative to control cells transduced with Renilla luciferase-targeting sgRNAs, cells transduced with RNF5-targeting sgRNAs showed impaired growth based on CellTiter-Glo luminescence assay (FIGS. 2H and 2I).

To further assess RNF5 function in AML, viability of xenografted patient-derived AML cells (PDX, AML-669) transduced with shRNF5 or control constructs was monitored. Using two independent shRNF5s (albeit limited KD efficiency), decreased viability of xenografted RNF5-KD cells relative to controls (FIGS. 2J-2I) was observed. These findings confirm the observations in AML cell lines and support the notion that RNF5 downregulation impairs proliferation of AML blasts.

### RNF5 Inhibition Enhances ER Stress-Induced Apoptosis of AML Cells

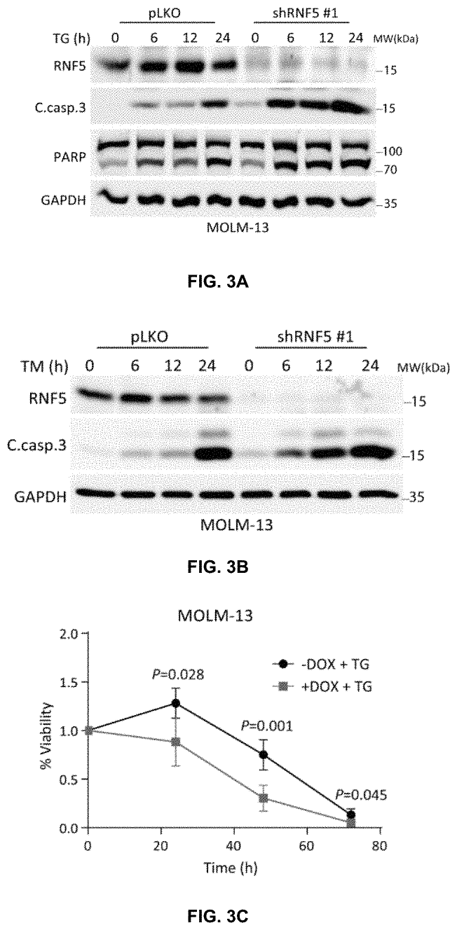

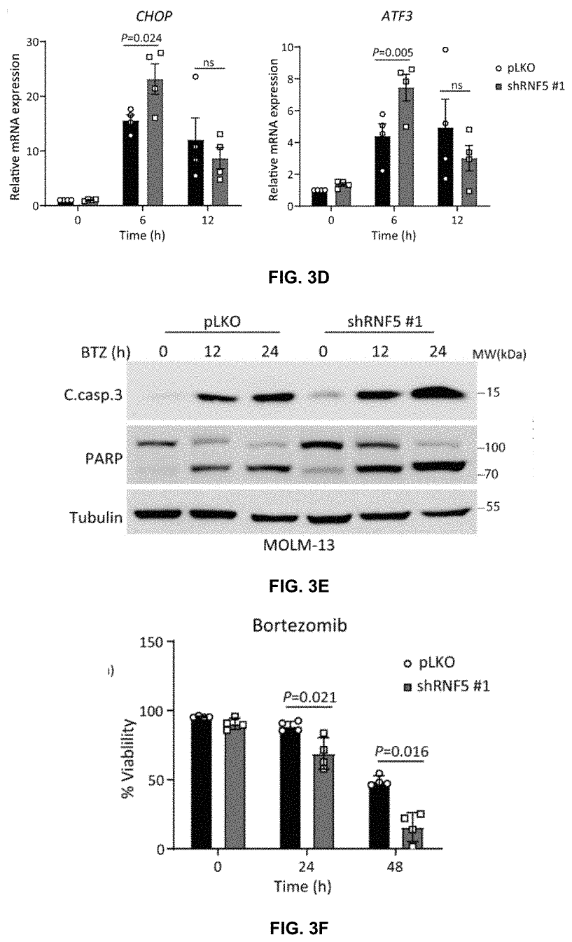

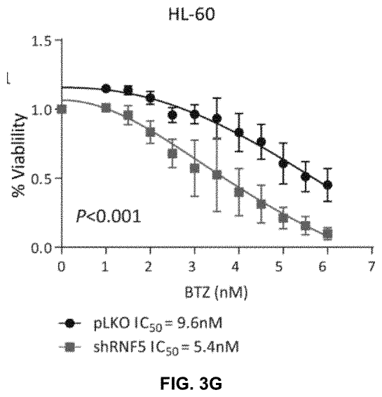

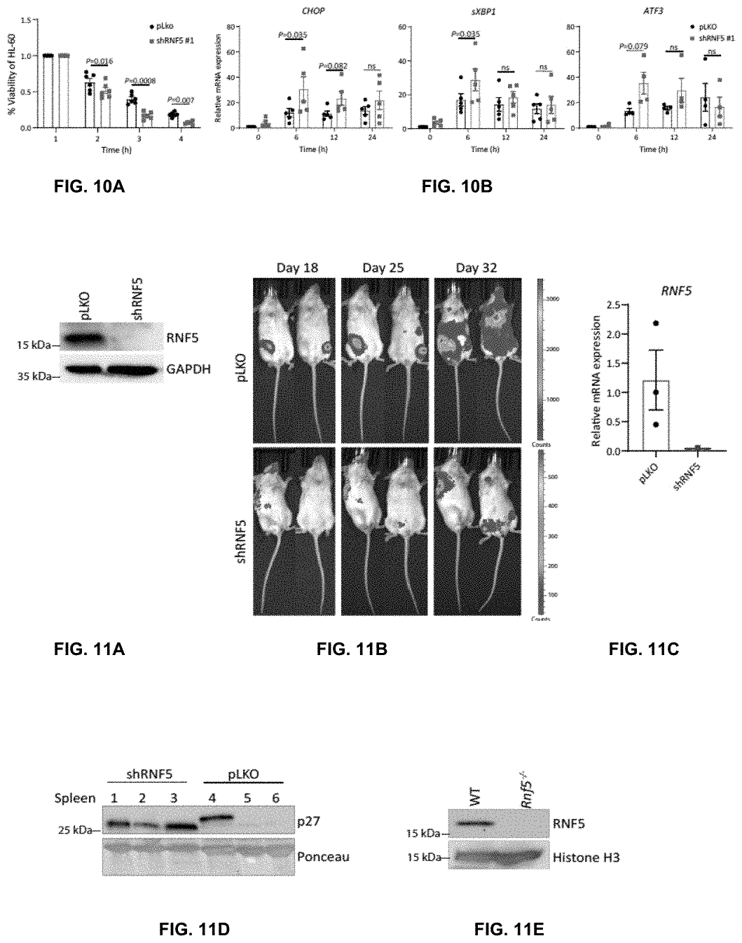

RNF5 functions as part of ERAD and the ER stress response. Changing RNF5 abundance alters the ER stress response in AML cells. RNF5-KD or control MOLM-13 cells were exposed to thapsigargin or tunicamycin to inhibit the ER Ca²⁺-ATPase (SERCA) or protein glycosylation, respectively, as a means to induce ER stress. Thapsigargin treatment of MOLM-13 RNF5-KD cells increased apoptotic markers to levels higher than those seen in control cells (FIGS. 3A and 3B). Thapsigargin treatment also decreased viability of MOLM-13 RNF5-KD cells to a greater extent than seen in control MOLM-13 WT RNF5 cells (FIG. 3C). Tunicamycin treatment also decreased viability of RNF5-KD HL-60 cells compared to tunicamycin-treated control HL-60 cells (FIG. 10A). Consistent with a function in ER stress, RNF5-KD increased levels of transcripts encoding key UPR components, including CHOP, ATF3, and sXBP1, in thapsigargin-treated MOLM-13 (FIG. 3D) and HL-60 cells (FIG. 10B), relative to mock-transduced controls.

Given the link between ER stress and proteasomal degradation, potential synergy between RNF5 KD and proteasomal inhibition was assessed. Indeed, RNF5-KD MOLM-13 cells treated with the proteasome inhibitor bortezomib (BTZ) showed increased levels of apoptotic markers such as cleaved forms of caspase-3 and PARP (FIG. 3E) and decreased viability (FIG. 3F) relative to control treated cells. Using annexin V and propidium iodide staining, which monitor degree of programmed cell death, RNF5-KD also enhanced apoptosis of BTZ-treated HL-60 cells (FIG. 3G), decreasing the BTZ IC₅₀ from 9.6 nM in controls to 5.4 nM in RNF5-KD cells. These data suggest that RNF5 plays a role in the response of AML cells to proteotoxic stress.

### RNF5 Loss Delays Leukemia Establishment and Progression

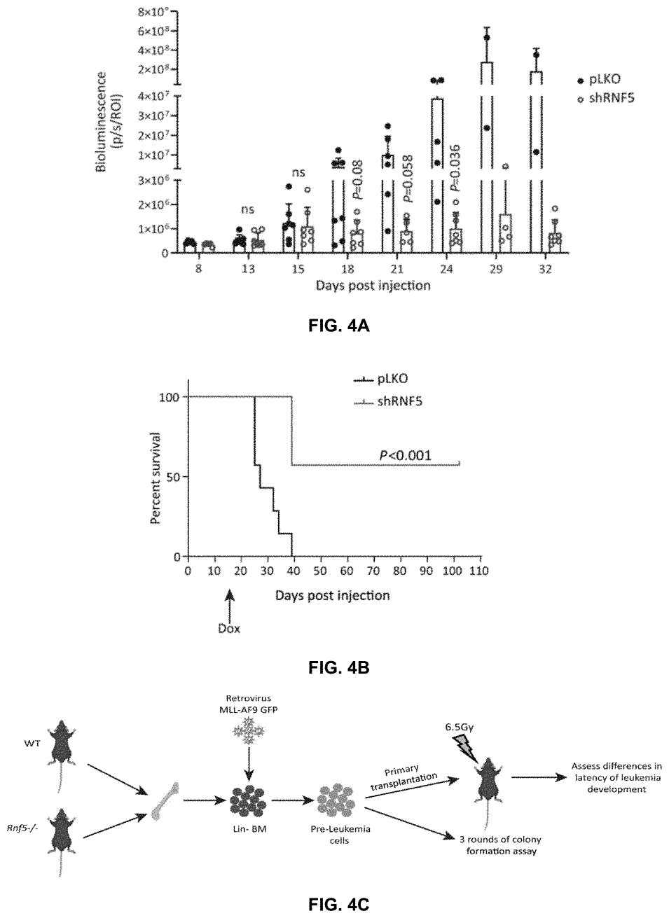

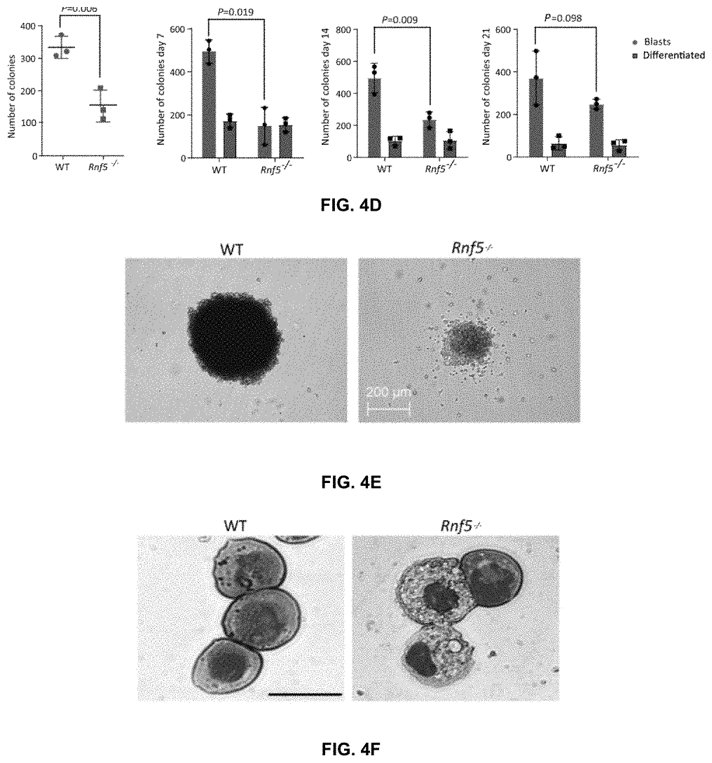

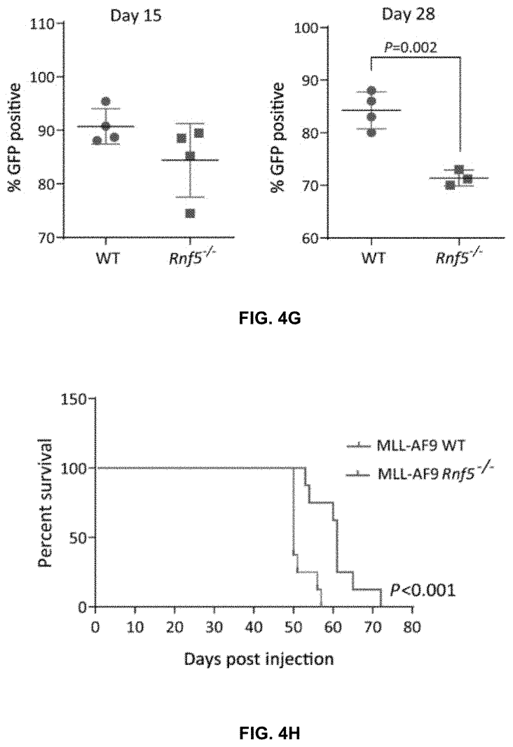

RNF5 activity modulates leukemia growth in vivo, as shown in a human AML xenograft model in which luciferase-expressing U937 cells (U937-pGFL) were transduced with doxycycline-inducible shRNF5 or control shRNA before being injected intravenously into NOD/SCID mice (FIG. 11A). Following leukemia establishment, as confirmed by bioluminescence, mice were fed a doxycycline-containing diet and monitored for disease progression and overall survival. Surprisingly, animals injected with RNF5-KD cells exhibited a markedly decreased leukemia burden and prolonged survival relative to control mice (FIGS. 4A, 4B, and 11B). RT-PCR analysis of splenocytes isolated from mice transplanted with RNF5 KD cells confirmed expression of shRNF5 (FIG. 11C). Western blot analysis of splenocyte lysates revealed more abundant expression of the cell cycle regulatory protein p27 in shRNF5 relative to control cells (FIG. 11D), consistent with in vitro data and with the delayed leukemia progression observed following RNF5 KD (FIGS. 4A and 4B). Collectively, these data indicate that RNF5 is required for AML cell proliferation in vivo.

RNF5 function in AML initiation was investigated using the MLL-AF9 model for in vitro and in vivo studies. The in vitro analysis used purified hematopoietic stem and progenitor (Lin-depleted) cells (HSPCs) from bone marrow of Rnf5^{-/-}, which exhibit normal development and hematopoiesis, and wild-type (WT) C57/BL6 mice. HSPCs from these mice were retrovirally transduced with a bicistronic construct harboring MLL-AF9 linked to a green fluorescent protein (GFP) marker. In assessing colony-forming capacity (CFC), compared to WT GFP-MLL-AF9 cells, Rnf5^{-/-} GFP-MLL-AF9 cells exhibited markedly reduced CFC in methylcellulose after 7, 14, and 21 days in culture and observed a striking reduction in the number of blast-like colonies (FIGS. 4D and 4E). These phenotypes are consistent with apparent terminal differentiation of Rnf5^{-/-} cells, as reflected by a greater cytoplasm/nucleus ratio and more vacuolated cytoplasm (FIGS. 4E and 4F).

To assess leukemogenesis in vivo, sub-lethally-irradiated WT C57/BL6 recipient mice were injected with GFP-MLL-AF9-transduced Rnf5^{WT} or Rnf5^{-/-} cells and monitored cell engraftment by flow cytometry for GFP-positive (GFP+) cells in peripheral blood (FIG. 4C). Analysis on days 15 and 28 post-injection identified fewer GFP+ cells in mice injected with GFP-MLL-AF9 Rnf5^{-/-} cells than in mice injected with GFP-MLL-AF9 Rnf5^{WT} cells, indicating a delay in leukemia development (FIG. 4G). Moreover, mice harboring GFP-MLL-AF9 Rnf5^{-/-} cells exhibited prolonged survival relative to mice injected with GFP-MLL-AF9 Rnf5^{WT} cells (FIG. 4H). Collectively, these data show that RNF5 loss decreases colony-forming capacity of MLL-AF9-transformed pre-leukemic cells in vitro and delays leukemia progression in vivo.

### RNF5 Activity Modulates Transcription in AML Cells

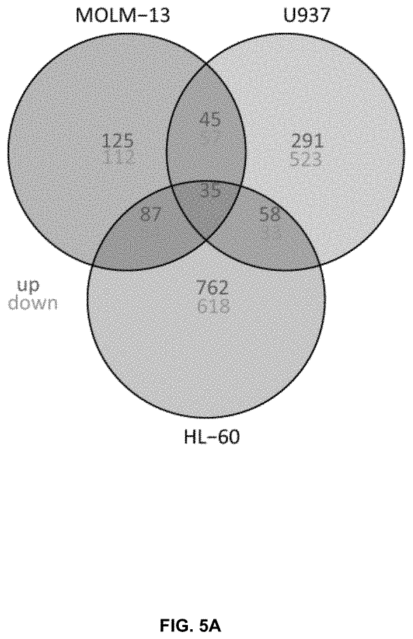

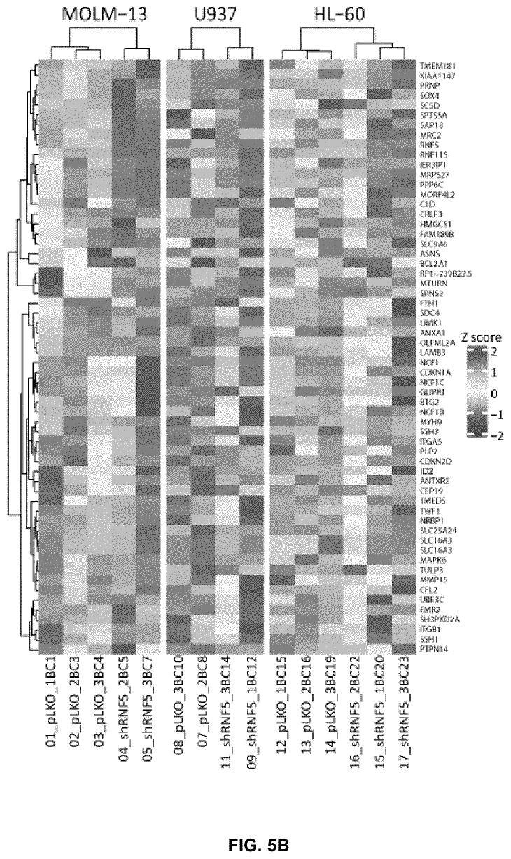

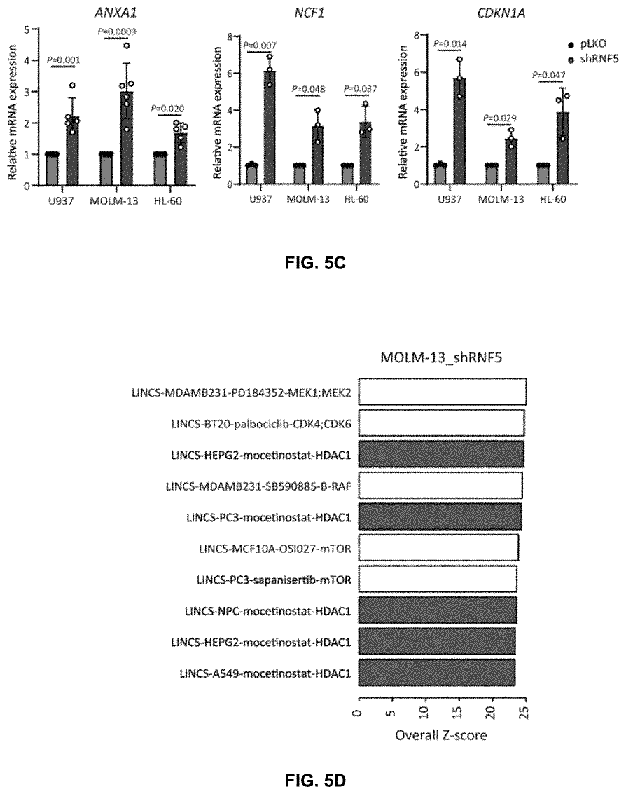

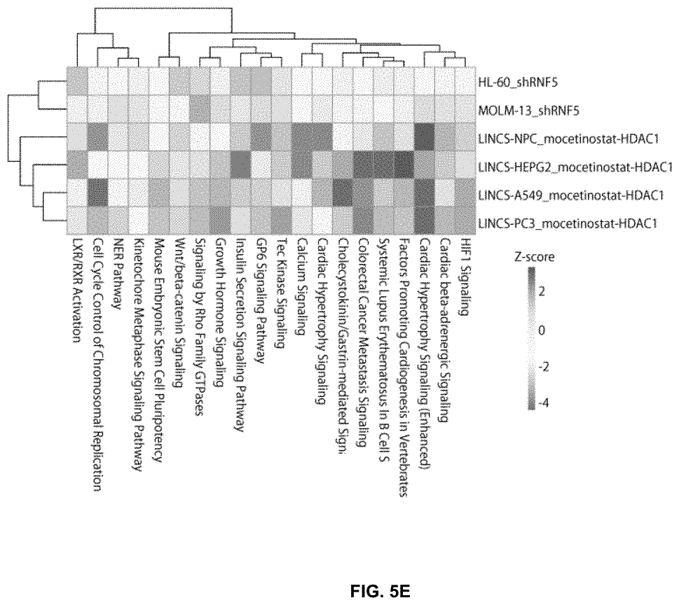

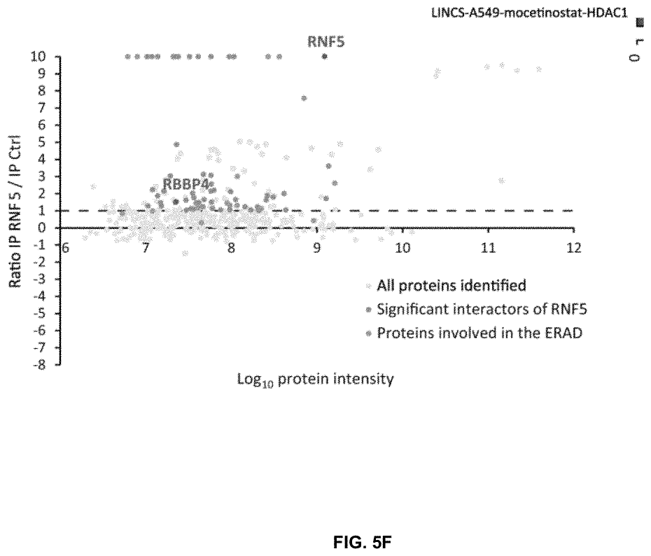

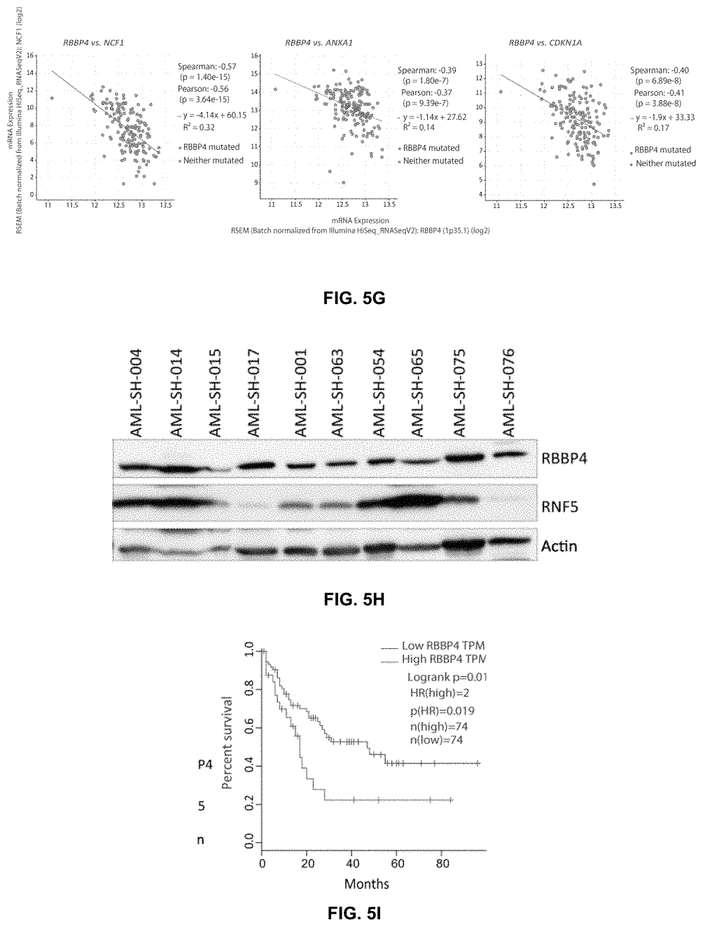

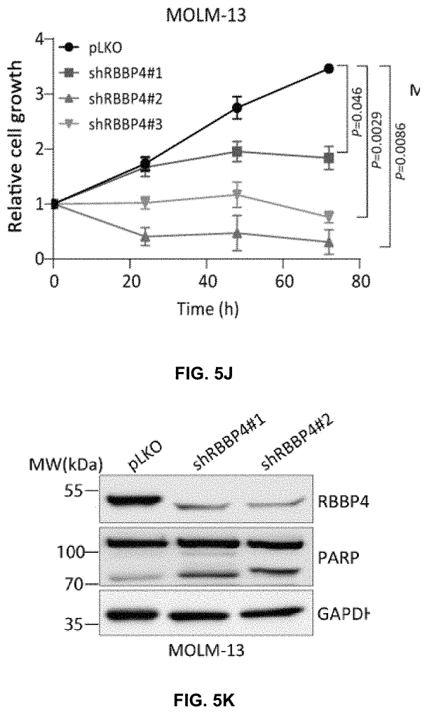

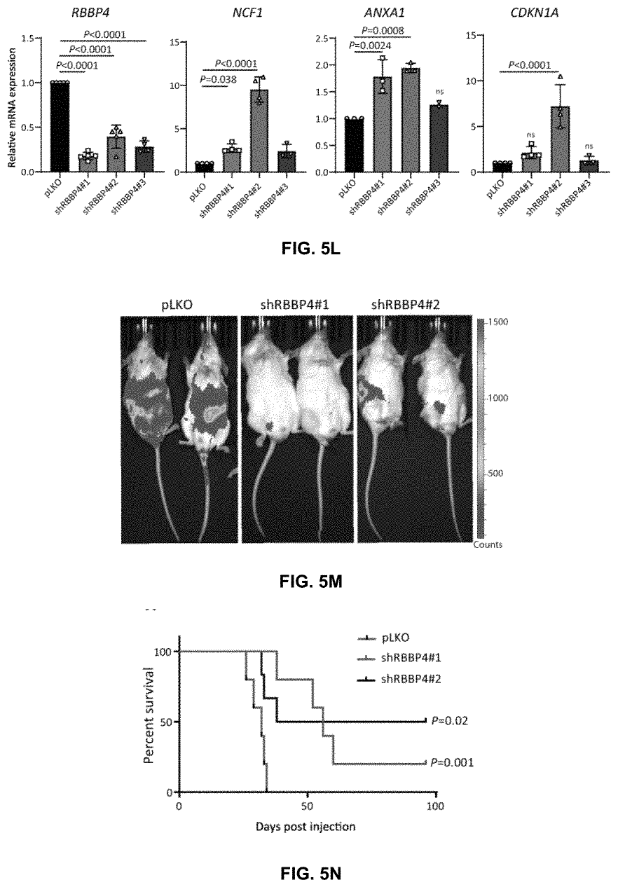

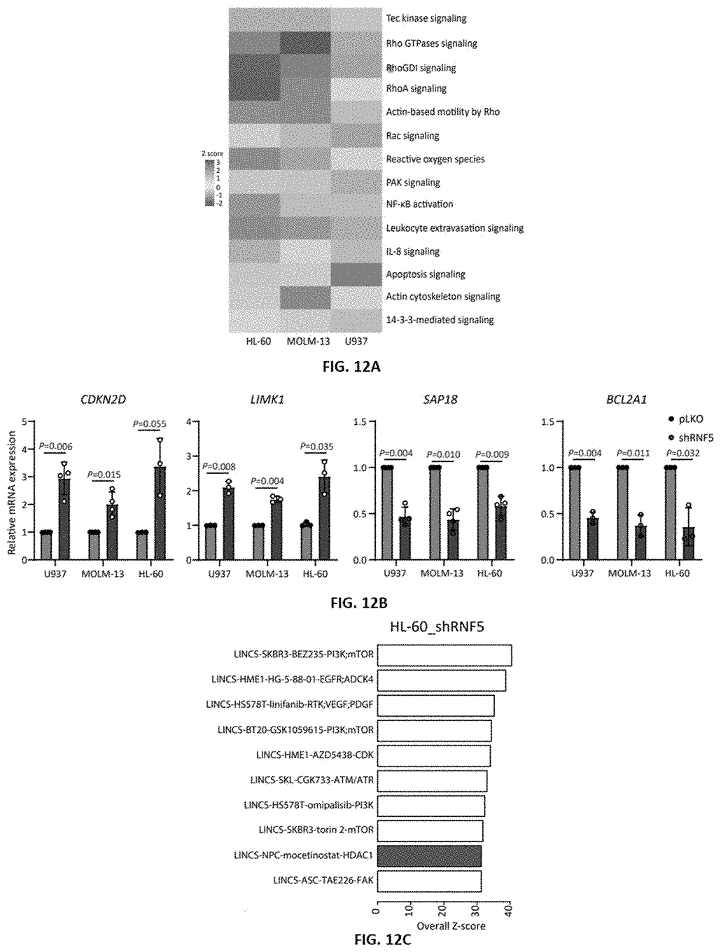

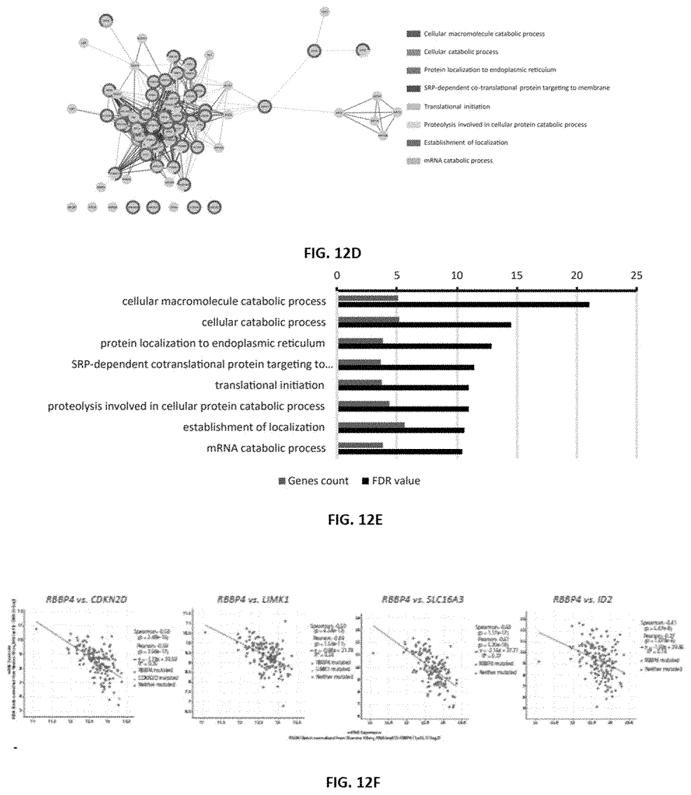

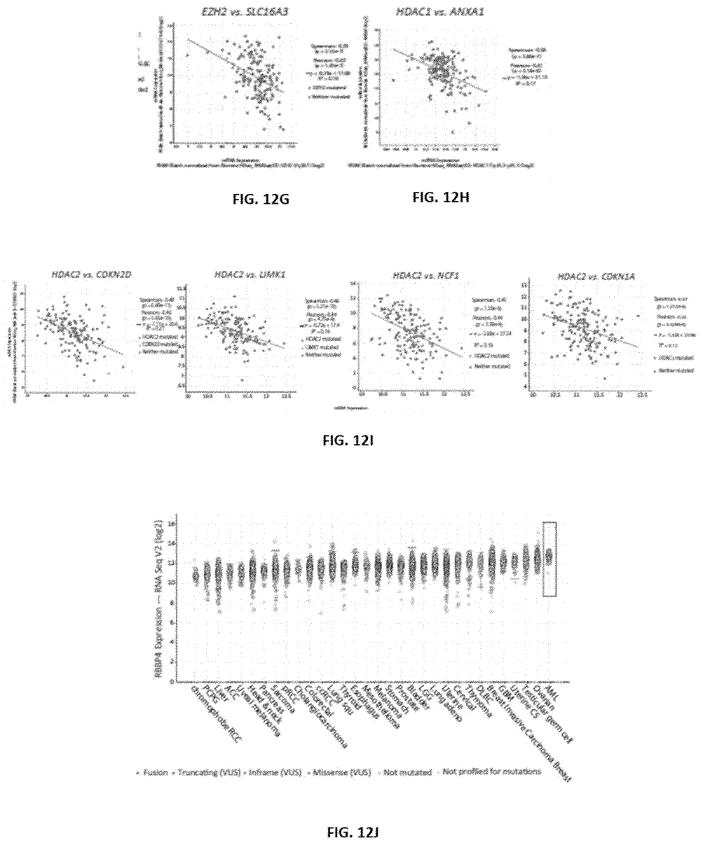

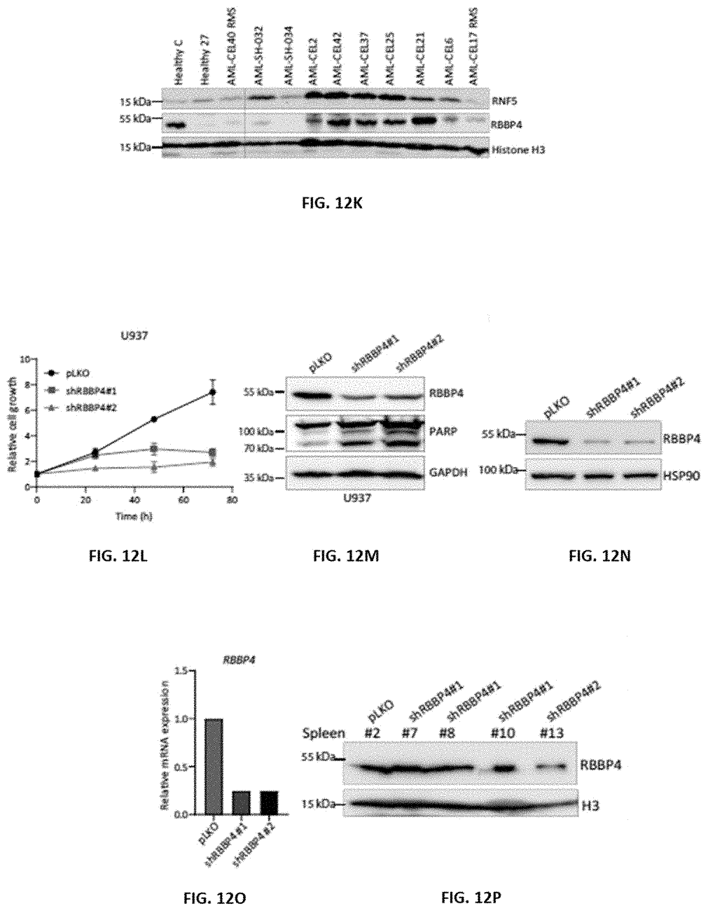

To identify pathways modulated by RNF5 activity in AML cells, transcriptional changes in MOLM-13, U937, and HL-60 AML lines expressing either RNF5-KD or control constructs were monitored. RNA sequencing (RNA-seq) analysis identified a total of 237, 814, and 1380 dysregulated genes in MOLM-13, U937 and HL-60, respectively, following RNF5 KD relative to control (RNF5-WT) cells (FIGS. 5A and 5B). Ingenuity Pathway Analysis identified selective enrichment of genes implicated in myeloid cell function such as NF-κB signaling, IL-8 signaling, reactive oxygen species, and several pathways related to cell migration such as Rho GTPase and Tec kinase signaling (FIG. 12A). Expression of a total of 59 genes (35 up- and 24 down-regulated) were significantly altered by RNSF KD in all three AML lines (FIGS. 5A and 5B). Among upregulated genes were CDKN1A and CDKN2D, which encode cell cycle inhibitors; LIMK1, which encodes a kinase functioning in regulation of the actin cytoskeleton; ANXA1, which encodes a calcium-binding protein functioning in metabolism, EGFR signaling and cell death programs; and NCF1, which encodes a subunit of NADPH oxidase (FIGS. 5C and 12B). Downregulated genes included antiapoptotic BCL2A1, and SAP18, which encodes a histone deacetylase complex subunit functioning in transcriptional repression (FIG. 12B). Moreover, such changes were consistent with phenotypic changes seen in RNF5-KD AML cell lines, such as reduced proliferation and increased apoptosis. Surprisingly, analysis of the Library of Integrated Network-Based Cellular Signatures (LINCS) drug screening database identified a notable overlap between transcriptomic changes induced by the HDAC1 inhibitor mocetinostat in various cancer cells and those seen in shRNF5-expressing MOLM-13 and HL-60 cells (FIG. 5D). Five and one out of top ten transcriptional changes identified in LINCS following HDAC inhibition overlapped with those seen following RNF5 KD in MOLM-13 and HL-60 cells, respectively (FIGS. 5D and 12C). Among commonly affected pathways were activation of GP6 and Rho GTPase signaling and repression of the nucleotide excision repair (NER) pathway (FIG. 5E). These observations suggest that RNF5 may regulate HDAC activity in AML cells.

RNF5 Interacts with and Ubiquitinates the Retinoblastoma Binding Protein 4

RNF5 elicits transcriptional changes through intermediate regulatory component(s). To identify RNF5-interacting proteins or substrates, liquid chromatography-tandem mass spectrometry (LC-MS/MS) was performed, and proteins immunoprecipitated from lysates of MOLM-13 cells expressing inducible Flag-tagged RNF5 were compared with those expressing empty vector. Among 65 RNF5-interacting proteins identified were previously reported substrates, such as 26S proteasome components, VCP and S100A8, as well as proteins implicated in AML development, such as DHX15 and gelsolin. Among the more abundant RNF5-bound proteins were components of ERAD, translation initiation, proteolytic and mRNA catabolic processes (FIGS. 5F, 12D and 12E).

Although none of the interacting proteins identified here were transcription factors, epigenetic modifications initiated by changes in RNF5 expression could also underlie changes in gene expression. In fact, one RNF5-interacting protein as the epigenetic regulator histone binding protein RBBP4 was identified (FIG. 5F). Analysis of transcriptome data from TCGA revealed an inverse correlation of RBBP4 expression with expression of genes upregulated in RNF5-KD cells (FIGS. 5G and 12F), suggesting that RNF5 positively controls RBBP4 transcriptional regulatory function. RBBP4 is a component of several chromatin assembly, remodeling, and nucleosome modification complexes, including PRC2 and the NuRD corepressor complex, which contains HDAC1 and HDAC2. Indeed, the inverse correlation between RBBP4 expression and RNF5-upregulated genes was mirrored when HDAC1, HDAC2 and EZH2 expression relative to RNF5-upregulated genes (FIGS. 12G-12I) were analyzed. Increased RBBP4 expression is also positively correlated with malignant phenotypes of several human tumors including AML. Analysis of tumor data in TCGA revealed high RBBP4 expression in AML, compared with other tumor types (FIG. 12J). Assessment of an AML patient cohort confirmed higher RBBP4 expression in samples from AML patients compared to healthy donors (FIGS. 5H and 12K). Stratification of AML patients based on RBBP4 expression indicated that high expression (the top 30%) correlated with poor overall survival (FIG. 5I).

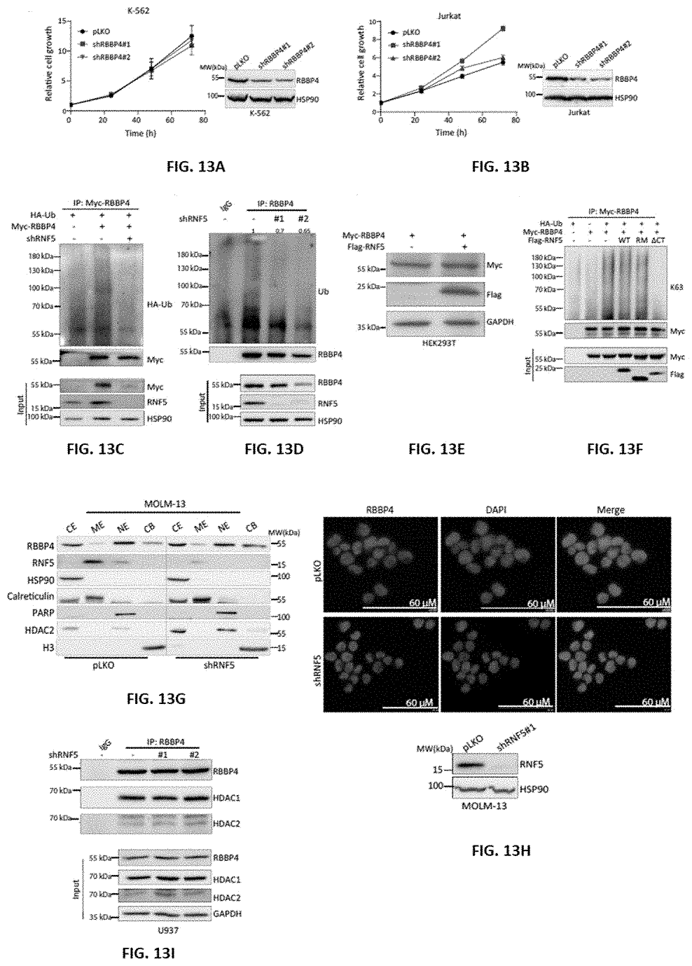

If RNF5 positively regulates RBBP4, RBBP4 KD should promote phenotypic changes in AML cells similar to RNF5 KD. Indeed, shRNA-based RBBP4 KD in MOLM-13 and U937 cells impaired their growth (FIGS. 5J and 12L), promoted PARP cleavage indicative of apoptosis (FIGS. 5K and 12M) and induced genes also induced by RNF5-KD (FIG. 5L). Furthermore, when examining RBBP4 function in vivo using the U937 xenograft model, mice harboring RBBP4-KD in xenografted cells showed delayed AML development and prolonged survival relative to WT-RBBP4 controls, phenocopying changes seen upon RNF5 KD (FIGS. 5M, 5N, 12N, and 12O). Notably, western blot analysis of cells from mice transplanted with RBBP4 KD cells showed that these cells retained RBBP4 expression (FIG. 12P). Since RBBP4 KD was confirmed prior to injection of these cells, it is likely that they emerged as escapers during in vivo selection (FIGS. 12N and 12O). The latter explains the shorter survival observed in mice that harbored the escapers, compared with mice that retained RBBP4 KD (FIG. 5N). Surprisingly, similar to outcomes seen in RNF5-KD AML cells, RBBP4-KD blocked growth of AML, but not CIVIL and T-ALL, cell lines (FIGS. 13A and 13B), confirming a link between RNF5 and RBBP4 in the context of AML.

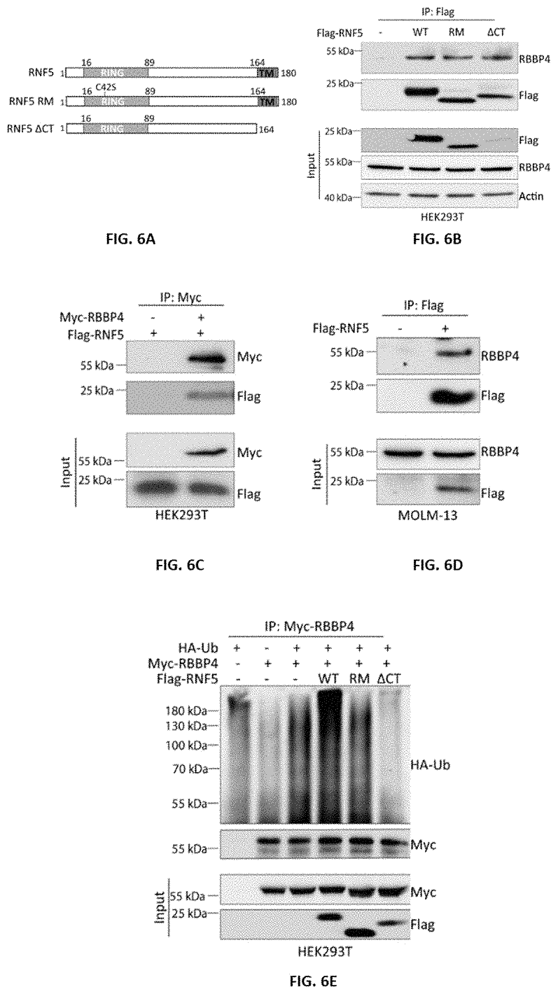

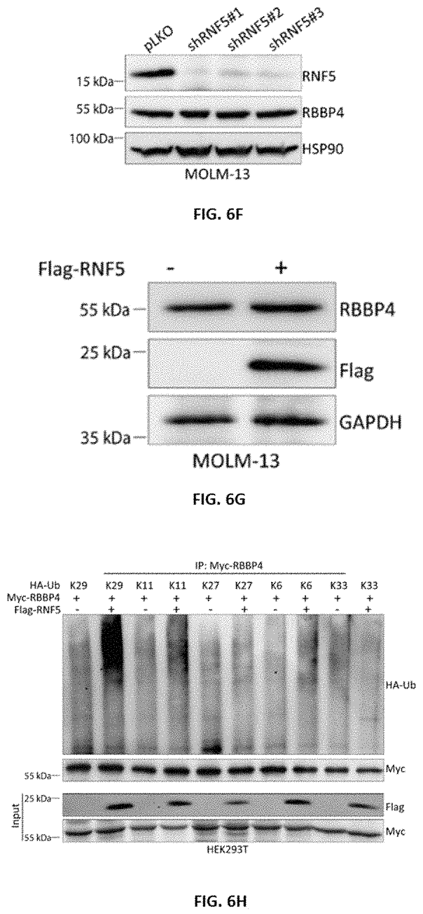

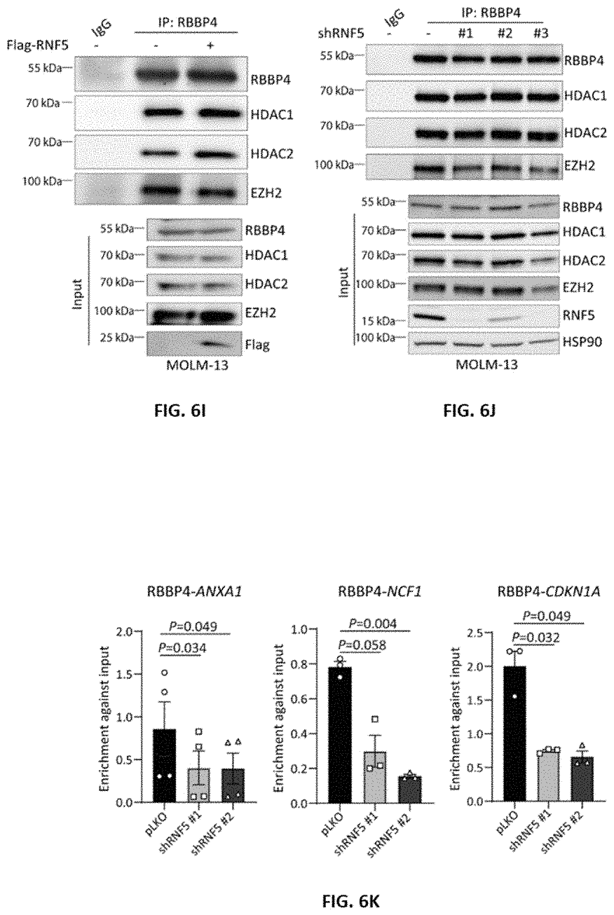

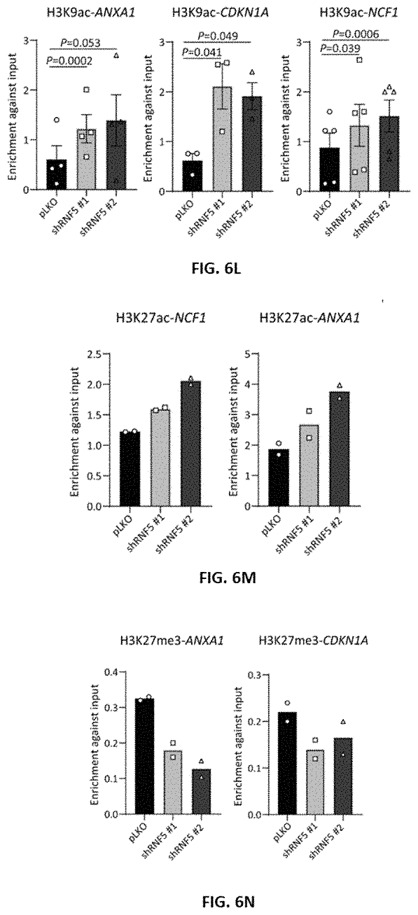

RNF5 is a transmembrane protein primarily associated with the ER, and its ubiquitin ligase domain is located in the cytosol. The interaction between RNF5 and RBBP4 in the HEK293T line was assessed by coimmunoprecipitation of ectopically-expressed WT RNF5, a catalytically inactive RING mutant (RNF5 RM), or a C-terminal transmembrane domain deletion mutant (RNF5 ACT) (FIG. 6A). Endogenous RBBP4 coimmunoprecipitated with all RNF5 constructs, suggesting that both the RING and transmembrane domains are dispensable for protein-protein interaction (FIG. 6B). Reciprocal IP using RBBP4 as bait confirmed interaction with RNF5 (FIG. 6C). Interaction between endogenous RBBP4 and ectopically expressed RNF5 was also confirmed in MOLM-13 cells (FIG. 6D). Next, the effects of RNF5 on RBBP4 ubiquitination was assessed. Co-expression of HA-tagged ubiquitin, Myc-tagged RBBP4, and Flag-tagged RNF5 constructs in HEK293T cells revealed that RBBP4 was ubiquitinated by WT RNF5, but not by RNF5 RM or RNF5 ACT (FIG. 6E), indicating that ubiquitin ligase activity (RING domain-dependent) and membrane association are both required for an RNF5-mediated increase in RBBP4 ubiquitination. Correspondingly, RNF5-KD in HEK293T or MOLM-13 cells decreased RBBP4 ubiquitination relative to controls (FIGS. 13C and 13D).

Notably, neither RNF5 overexpression nor RNF5 KD altered abundance of RBBP4 protein, suggesting that RBBP4 ubiquitination by RNF5 does not occur via formation of proteasome-targeting K48 ubiquitin chains and does not alter RBBP4 stability (FIGS. 6F, 6G, and 13E). Immunoprecipitation of RBBP4 and immunoblot using an antibody specific for the K63 chain topology revealed no notable differences in cells overexpressing any form of RNF5, suggesting that RNF5 does not induce K63 ubiquitination of RBBP4 (FIG. 13F). The linkage-specific polyubiquitin induced by RNF5 on RBBP4 was assessed by using mutant HA-ubiquitin constructs with only one lysine available for linkage (K-only mutants: K29, K11, K27, K6, and K33) in which all lysine residues except that indicated are mutated to arginine allowing a single type of homotypic chain. Changes in Myc-tagged RBBP4 ubiquitination in cells overexpressing Flag-tagged RNF5 were monitored. Poly-ubiquitination of RBBP4 was enhanced by RNF5 only in the presence of K29 ubiquitin (FIG. 6H), strongly suggesting that RNF5 induces K29-topology polyubiquitination of RBBP4.

### RNF5 Promotes Recruitment of RBBP4 to Gene Promoters

Because RNF5 activity does not alter RBBP4 stability, the next question to ask is whether RNF5 affects RBBP4 localization or interactions with other proteins. Subcellular fractionation in MOLM-13 cells and immunofluorescent analyses of nuclear and chromatin bound RBBP4 did not identify changes in RBBP4 localization following RNF5 KD (FIGS. 13G and 13H). Since RBBP4 is a component of PRC2 and complexes containing HDAC, the next question to ask is whether RNF5 activity alters formation of these complexes or their recruitment to target gene promoters. Neither overexpression nor KD of RNF5 affected RBBP4 interaction with HDAC1, HDAC2, or EZH2 (FIGS. 6I, 6J, and 13I), suggesting that RBBP4 ubiquitination by RNF5 is not required for assembly of RBBP4-containing these complexes.

Then, chromatin immunoprecipitation (ChIP) and quantitative PCR (qPCR) were used to investigate RBBP4 recruitment to promoters of genes regulated by either RNF5 or RBBP4. RNF5 KD decreased RPPB4 recruitment to ANXA1, NCF1, and CDKN1A promotors (FIG. 6K). Examination of histone modifications at promoters of these genes identified that RNF5 KD increased H3K9 and H3K27 acetylation (FIGS. 6L and 6M) and reduced H3K27 methylation (FIG. 6N), changes indicative of increased gene expression. These changes are consistent with their increased expression seen following RNF5 KD (FIG. 5C) and suggest that RNF5 control of gene expression in AML cells is mediated by RBBP4.

### RNF5 Inhibition Sensitizes AML Cells to HDAC Inhibitors

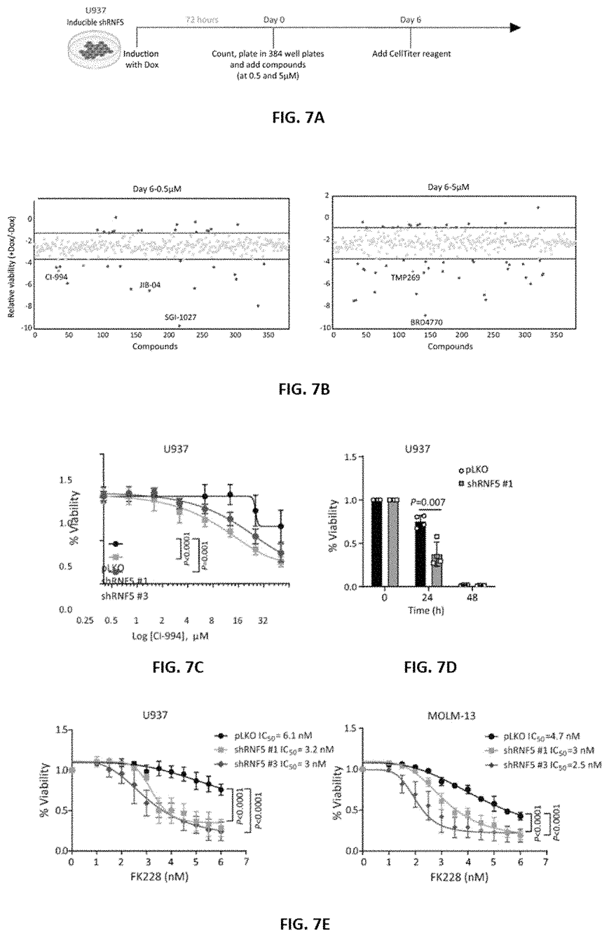

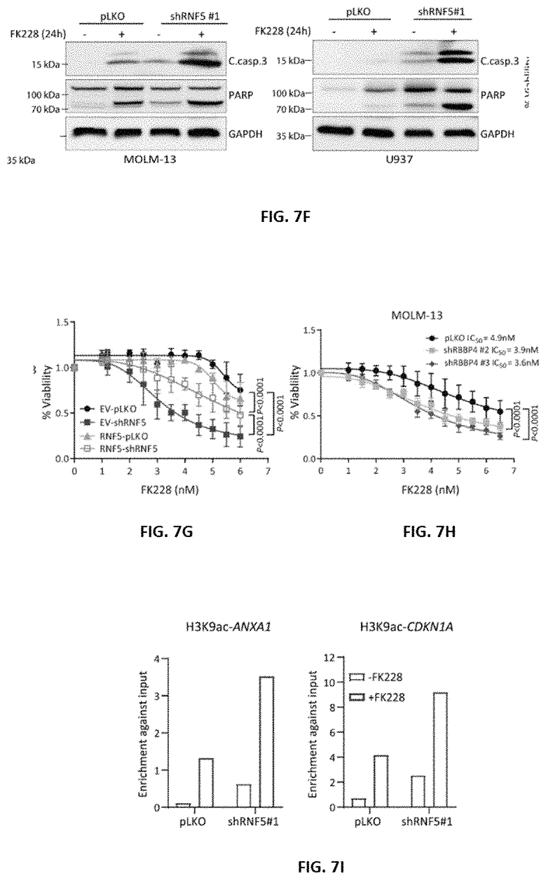

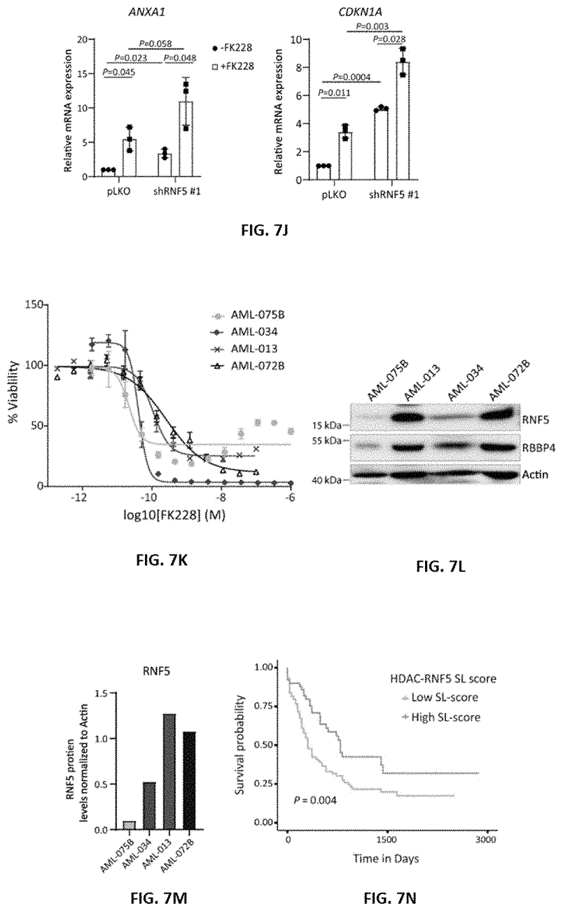

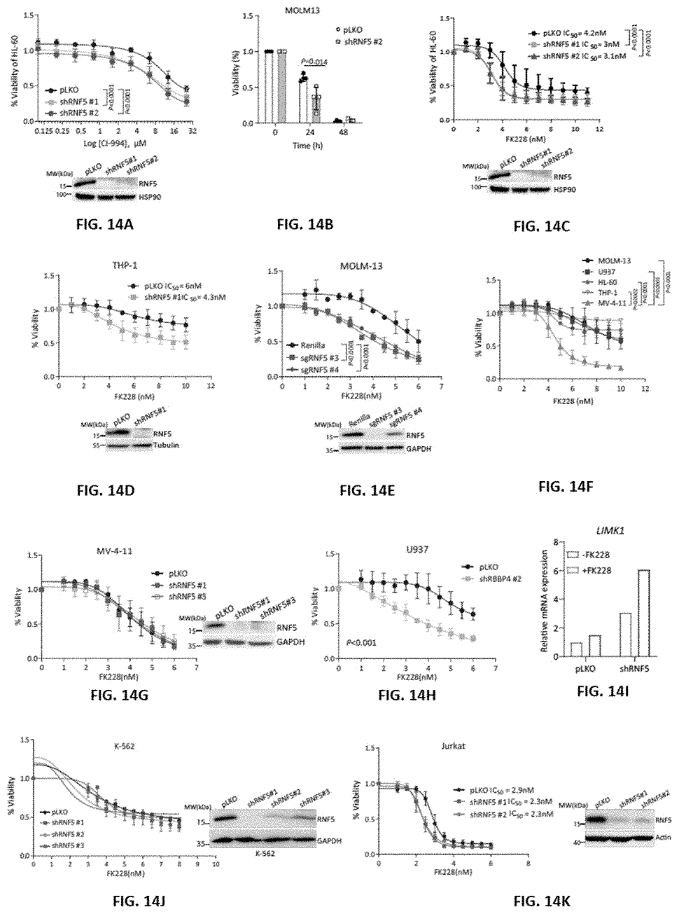

As independent support for the function of the RNF5-RBBP4 regulatory axis in promoting AML cell growth, synergistic interactions between RNF5 and epigenetic modulators were screened. To do so, the effect of 261 epigenetic inhibitors at two concentrations (See Table 2) was assessed on growth of U937 cells that stably express inducible shRNF5 (FIGS. 7A and 7B). Of epigenetic inhibitors tested, 49 decreased viability of shRNF5-expressing cells relative to control WT RNF5 AML cells (FIG. 7B). Among the 49 inhibitors were several hypomethylation agents, including several histone methyltransferases (such as G9a), histone demethylases (such as Jumonji histone demethylases) and HDAC inhibitors (such as TMP269, pimelic diphenylamide 106, and N-acetyldinaline [CI-994]). Because RBBP4 is a key component of the HDAC complex and given that RNF5 KD induces transcriptional changes comparable to HDAC1 inhibition (FIGS. 5D and 5E), possible synergy between RNF5 inhibition and HDAC inhibitors was assessed. To do so, HDAC inhibitor CI-994 was selected, which is in clinical trials against several cancers (https://www.drugbank.ca/drugs/DB12291), for additional validation. Indeed, U937 and HL-60 cells subjected to RNF5-KD exhibited a lower IC₅₀ for CI-994 in terms of cell viability relative to control cells (FIGS. 7C and 14A), suggesting that RNF5 KD sensitizes AML cells to HDAC inhibition.

TABLE 2
List of small molecule epigenetic modulators used to identify
possible synergy with RNF5 knockdown in AML cells

| Molecule Name | Bio-Activity | CAS Number |
|---|---|---|
| B2 | SIRT2 inhibitor | 115687-05-3 |
| Valproic acid | HDAC inhibitor | 99-66-1 |
| Piceatannol | SIRT activator | 10083-24-6 |
| Resveratrol | SIRT1 activator | 501-36-0 |
| Suramin·6Na | SIRT1 inhibitor | 129-46-4 |
| Triacetylresveratrol | SIRT1 activator | 42206-94-0 |
| Phenylbutyrate·Na | HDAC inhibitor | 1716-12-7 |
| NSC-3852 | HDAC inhibitor | 3565-26-2 |
| Nicotinamide | SIRT inhibitor | 98-92-0 |
| BML-266 | SIRT2 inhibitor | 96969-83-4 |
| AGK2 | SIRT2 inhibitor | 304896-28-4 |
| BIX-01294 | Histone methyl transferase inhibitor | 935693-62-2 |
| SAHA | HDAC inhibitor | 149647-78-9 |
| Anacardic acid | HAT inhibitor | 16611-84-0 |
| 5-Aza-2′- | DNA Me transferase inhibitor | 2353-33-5 |
| deoxycytidine | | |
| M-344 | HDAC inhibitor | 251456-60-7 |
| ITSA-1 | Inhibitor of TSA activity | 200626-61-5 |
| Scriptaid | HDAC inhibitor | 287383-59-9 |
| EX-527 | SIRT1 inhibitor | 49843-98-3 |
| Salermide | SIRT inhibitor | 1105698-15-4 |
| CI-994 | HDAC inhibitor | 112522-64-2 |
| BML-210 | HDAC inhibitor | 537034-17-6 |
| Tranylcypromine | Lysine demethylase inhibitor | 13492-01-8 |
| hemisulfate | | (H2SO4) |
| Trichostatin A | HDAC inhibitor | 58880-19-6 |
| 2,4- | Histone demethylase inhibitor | 499-80-9 |
| Pyridinedicarboxylic | | |
| Acid | | |
| Garcinol | HAT inhibitor | 78824-30-3 |
| Splitomicin | SIRT-2 inhibitor | 3/9/5690 |
| Apicidin | HDAC inhibitor | 183506-66-3 |
| Suberoyl bis- | HDAC inhibitor | 38937-66-5 |
| hydroxamic acid | | |
| Nullscript | Scriptaid Neg control | 300816-11-9 |
| Zebularine | DNA Me transferase inhibitor | 10/6/3690 |
| Isonicotinamide | nicotinamide antagonist | 1453-82-3 |
| Fluoro-SAHA | HDAC inhibitor | 149648-08-8 |
| Valproic acid | HDAC inhibitor | 106132-78-9 |
| hydroxamate | | |
| MC-1293 | HDAC inhibitor | 117378-93-5 |
| Butyrolactone 3 | HAT inhibitor | 778649-18-6 |
| CTPB | HAT inhibitor | 586976-24-1 |
| Oxamflatin | HDAC inhibitor | 151720-43-3 |
| Sirtinol | SIRT inhibitor | 410536-97-9 |
| BML-278 | SIRT1 actvator | 120533-76-8 |
| NCH-51 | HDAC inhibitor | 848354-66-5 |
| Aminoresveratrol | SIRT1 activator | 1224713-76-1 |
| sulfate | | |
| BML-281 | HDAC-6 inhibitor | 1045792-66-2 |
| Droxinostat | Droxinostat (CMH, 5809354) is a selective inhibitor of HDAC, | 99873-43-5 |
| | mostly for HDACs 6 and 8 with IC50 of 2.47 uM and 1.46 uM, | |
| | greater than 8-fold selective against HDAC3 and no inhibition to | |
| | HDAC1, 2, 4, 5, 7, 9, and 10. | |
| Azacitidine | Azacitidine is a nucleoside analogue of cytidine that | 320-67-2 |
| | specifically inhibits DNA methylation by trapping DNA | |
| | methyltransferases. | |
| INO-1001 (3- | INO-1001 is a potent inhibitor of PARP with IC50 of <50 nM | 3544-24-9 |
| Aminobenzamide) | in CHO cells and a mediator of oxidant-induced myocyte | |
| | dysfunction during reperfusion. Phase 2. | |
| 2-Methoxyestradiol | 2-Methoxyestradiol depolymerizes microtubules and blocks | 362-07-2 |
| (2-MeOE2) | HIF-1alpha nuclear accumulation and HIF-transcriptional | |
| | activity. Phase 2. | |
| Procainamide HCl | Procainamide HCl is a sodium channel blocker, and | 614-39-1 |
| | also a DNA methyltransferase inhibitor, used in the | |
| | treatment of cardiac arrhythmias. | |
| Quercetin | Quercetin is a natural flavonoid present in vegetables, fruit | 117-39-5 |
| | and wine and is a PI3K inhibitor with IC50 of 2.4-5.4 uM. | |
| AG-490 (Tyrphostin | AG-490 (Tyrphostin B42) is an inhibitor of EGFR with | 133550-30-8 |
| B42) | IC50 of 0.1 uM, 135-fold more selective for EGFR versus | |
| | ErbB2, also inhibits JAK2 with no activity to Lek, Lyn, Btk, | |
| | Syk and Src. | |
| RG108 | RG108 is an inhibitor of DNA methyltransferase with IC50 of | 48208-26-0 |
| | 115 nM, does not cause trapping of covalent enzymes. | |
| WHI-P154 | WHI-P154 is a potent JAK3 inhibitor with IC50 of 1.8 uM, no | 211555-04-3 |
| | activity against JAK1 or JAK2, also inhibits EGFR, Src, Abl, | |
| | VEGFR and MAPK, prevents Stat3, but not Stat5 | |
| | phosphorylation. | |
| JNJ-7706621 | JNJ-7706621 is pan-CDK inhibitor with the highest | 443797-96-4 |
| | potency on CDK1/2 with IC50 of 9 nM/4 nM and showing >6- | |
| | fold selectivity for CDK1/2 than CDK3/4/6. It also | |
| | potently inhibits Aurora A/B and has no activity on Plk1 | |
| | and Wee1. | |
| PJ34 | PJ-34 is a PARP inhibitor with EC50 of 20 nM and is equally | 344458-19-1 |
| | potent to PARP1/2. | |
| WP1066 | WP1066 is a novel inhibitor of JAK2 and STAT3 with IC50 | 857064-38-1 |
| | of 2.30 uM and 2.43 uM in HEL cells; shows activity to | |
| | JAK2, STAT3, STAT5, and ERK1/2 not JAK1 and JAK3. | |
| Entinostat (MS-275) | Entinostat (MS-275) strongly inhibits HDAC1 and HDAC3 | 209783-80-2 |
| | with IC50 of 0.51 uM and 1.7 uM, compared with HDACs 4, | |
| | 6, 8, and 10. Phase 1/2. | |
| Mocetinostat | Mocetinostat (MGCD0103) is a potent HDAC inhibitor with | 726169-73-9 |
| (MGCD0103) | most potency for HDAC1 with IC50 of 0.15 μM, 2- to 10- | |
| | fold selectivity against HDAC2, 3, and 11, and no activity to | |
| | HDAC4, 5, 6, 7, and 8. Phase 1/2. | |
| Belinostat (PXD101) | Belinostat (PXD101) is a novel HDAC inhibitor with IC50 of | 414864-00-9 |
| | 27 nM, with activity demonstrated in cisplatin-resistant tumors. | |
| | Phase 1/2. | |
| Panobinostat | Panobinostat (LBH589) is a novel broad-spectrum HDAC | 404950-80-7 |
| (LBH589) | inhibitor with IC50 of 5 nM. Phase 3. | |
| Entacapone | Entacapone inhibits catechol-O-methyltransferase(COMT) | 130929-57-6 |
| | with IC50 of 151 nM. | |
| Alisertib | Alisertib (MLN8237) is a selective Aurora A inhibitor with | 1028486-01-2 |
| (MLN8237) | IC50 of 1.2 nM. It has >200-fold higher selectivity for | |
| | Aurora A than Aurora B. Phase 3. | |
| Romidepsin (FK228, | Romidepsin (FK228, depsipeptide) is a potent HDAC1 | 128517-07-7 |
| Depsipeptide) | and HDAC2 inhibitor with IC50 of 36 nM and 47 nM, | |
| | respectively. | |
| S-Ruxolitinib | S-Ruxolitinib is the chirality of INCB018424, which is the first | 941678-49-5 |
| (INCB018424) | potent, selective, JAK1/2 inhibitor to enter the clinic with IC50 | |
| | of 3.3 nM/2.8 nM, >130-fold selectivity for JAK1/2 versus | |
| | JAK3. Phase 3. | |
| ZM 447439 | ZM 447439 is a selective and ATP-competitive inhibitor for | 331771-20-1 |
| | Aurora A and Aurora B with IC50 of 110 nM and 130 nM, | |
| | respectively. It is more than 8-fold selective for Aurora A/B | |
| | than MEK1, Src, Lck and has little effect againstCDK1/2/4, | |
| | [symbol] | |
| VX-680 (Tozasertib, | VX-680 (Tozasertib, MK-0457) is a pan-Aurora inhibitor, | 639089-54-6 |
| MK-0457) | mostly against Aurora A with Kiapp of 0.6 nM, less potent | |
| | towards Aurora B/Aurora C and 100-fold more selective for | |
| | Aurora A than 55 other kinases. Phase 2. | |
| Danusertib (PHA- | Danusertib (PHA-739358) is an Aurora kinase inhibitor for | 827318-97-8 |
| 739358) | Aurora A/B/C with IC50 of 13 nM/79 nM/61 nM, modestly | |
| | potent to Abl, TrkA, c-RET and FGFR1, and less potent to | |
| | Lck, VEGFR2/3, c-Kit, CDK2, etc. Phase 2. | |
| AT9283 | AT9283 is a potent JAK2/3 inhibitor with IC50 of 1.2 nM/1.1 | 896466-04-9 |
| | nM; also potent to Aurora A/B, Abl(T315I). Phase 1/2. | |
| Barasertib | AZD1152-HQPA (Barasertib) is a highly selective Aurora B | 722544-51-6 |
| (AZD1152-HQPA) | inhibitor with IC50 of 0.37 nM, ~100 fold more selective for | |
| | Aurora B over Aurora A. | |
| SNS-314 Mesylate | SNS-314 Mesylate is a potent and selective inhibitor of | 1146618-41-8 |
| | Aurora A, Aurora B and Aurora C with IC50 of 9 nM, 31 | |
| | nM, and 3 nM, respectively. It is less potent to Trk A/B, | |
| | Flt4, Fms, Axl, c-Raf and DDR2. Phase 1. | |
| CYC116 | CYC116 is a potent inhibitor of Aurora A/B with Ki of 8.0 | 693228-63-6 |
| | nM/9.2 nM, is less potent to VEGFR2 (Ki of 44 nM), with 50- | |
| | fold greater potency than CDKs, not active against PKA, | |
| | Akt/PKB, PKC, no effect on GSK-3alpha/beta, CK2, Plk1 and | |
| | SAPK2A. Phase 1. | |
| ENMD-2076 | ENMD-2076 has selective activity against Aurora A and Flt3 | 1291074-87-7 |
| | with IC50 of 14 nM and 1.86 nM, 25-fold selective for | |
| | Aurora A than over Aurora B and less potent to | |
| | VEGFR2/KDR and VEGFR3, FGFR1 and FGFR2 and | |
| | PDGFRalpha. Phase 2. | |
| Aurora A Inhibitor I | Aurora A Inhibitor I is a novel, potent, and selective | 1158838-45-9 |
| | inhibitor of Aurora A with IC50 of 3.4 nM. It is 1000-fold | |
| | more selective for Aurora A than Aurora B. | |
| PHA-680632 | PHA-680632 is potent inhibitor of Aurora A, Aurora B and | 398493-79-3 |
| | Aurora C with IC50 of 27 nM, 135 nM and 120 nM, | |
| | respectively. It has 10- to 200-fold higher IC50 for FGFR1, | |
| | FLT3, LCK, PLK1, STLK2, and VEGFR2/3. | |
| CCT129202 | CCT129202 is an ATP-competitive pan-Aurora inhibitor for | 942947-93-5 |
| | Aurora A, Aurora B and Aurora C with IC50 of 0.042 uM, | |
| | 0.198 uM and 0.227 uM, respectively. It is less potent to | |
| | FGFR3, GSK3beta, PDGFRbeta, etc. | |
| Hesperadin | Hesperadin potently inhibits Aurora B with IC50 of 250 | 422513-13-1 |
| | nM. It markedly reduces the activity of AMPK, Lck, | |
| | MKK1, MAPKAP-K1, CHK1 and PHK while it does not | |
| | inhibit MKK1 activity in vivo. | |
| NVP-BSK805 2HCl | NVP-BSK805 is a potent and selective ATP-competitive | 1092499-93-8 |
| | JAK2 inhibitor with IC50 of 0.5 nM, >20-fold selectivity | (free base) |
| | towards JAK1, JAK3 and TYK2. | |
| KW-2449 | KW-2449 is a multiple-targeted inhibitor, mostly for Flt3 with | 1000669-72-6 |
| | IC50 of 6.6 nM, modestly potent to FGFR1, Bcr-Abl and | |
| | Aurora A; little effect on PDGFRβ, IGF-1R, EGFR. Phase 1. | |
| LY2784544 | LY2784544 is a potent JAK2 inhibitor with IC50 of 3 nM, | 1229236-86-5 |
| | effective in JAK2V617F, 8- and 20-fold selective versus JAK1 | |
| | and JAK3. Phase 2. | |
| AZ 960 | AZ 960 is a novel ATP competitive JAK2 inhibitor with IC50 | 905586-69-8 |
| | and Ki of <3 nM and 0.45 nM, 3-fold selectivity of AZ960 for | |
| | JAK2 over JAK3. | |
| CYT387 | CYT387 is an ATP-competitive inhibitor of JAK1/JAK2 | 1056634-68-4 |
| | with IC50 of 11 nM/18 nM, ~10-fold selectivity versus | |
| | JAK3. Phase 1/2. | |
| Tofacitinib (CP- | Tofacitinib citrate (CP-690550 citrate) is a novel inhibitor of | 540737-29-9 |
| 690550, Tasocitinib) | JAK3 with IC50 of 1 nM, 20- to 100-fold less potent against | |
| | JAK2 and JAK1. | |
| TAK-901 | TAK-901 is a novel inhibitor of Aurora A/B with IC50 of 21 | 934541-31-8 |
| | nM/15 nM. It is not a potent inhibitor of cellular JAK2, c-Src or | |
| | Abl. Phase 1. | |
| TG101209 | TG101209 is a selective JAK2 inhibitor with IC50 of 6 nM, | 936091-14-4 |
| | less potent to Flt3 and RET with IC50 of 25 nM and 17 nM, ~30- | |
| | fold selective for JAK2 than JAK3, sensitive to JAK2V617F | |
| | and MPLW515L/K mutations. | |
| AMG-900 | AMG 900 is a potent and highly selective pan-Aurora kinases | 945595-80-2 |
| | inhibitor for Aurora A/B/C with IC50 of 5 nM/4 nM/1 nM. It | |
| | is >10-fold selective for Aurora kinases > p38 > Tyk2 > | |
| | JNK2 > Met > Tie2. Phase 1. | |
| MLN8054 | MLN8054 is a potent and selective inhibitor of Aurora A | 869363-13-3 |
| | with IC50 of 4 nM. It is more than 40-fold selective for | |
| | Aurora A than Aurora B. Phase 1. | |
| Baricitinib | Baricitinib is a selective JAK1 and JAK2 inhibitor with | 1187594-09-7 |
| (LY3009104, | IC50 of 5.9 nM and 5.7 nM, ~70 and ~10-fold selective | |
| INCB028050) | versus JAK3 and Tyk2, no inhibition to c-Met and Chk2. | |
| TG101348 | TG-101348 (SAR302503) is a selective inhibitor of JAK2 | 936091-26-8 |
| (SAR302503) | with IC50 of 3 nM, 35- and 334-fold more selective for | |
| | JAK2 versus JAK1 and JAK3. Phase 1/2. | |
| MK-5108 (VX-689) | MK-5108 (VX-689) is a highly selective Aurora A | 1010085-13-8 |
| | inhibitor with IC50 of 0.064 nM and is 220- and 190-fold | |
| | more selective for Aurora A than Aurora B/C, while it | |
| | inhibits TrkA with less than 100-fold selectivity. Phase 1. | |
| CCT137690 | CCT137690 is a highly selective inhibitor of Aurora A, | 1095382-05-0 |
| | Aurora B and Aurora C with IC50 of 15 nM, 25 nM and 19 | |
| | nM. It has little effect on hERG ion-channel. | |
| CEP-33779 | CEP33779 is a selective JAK2 inhibitor with IC50 of 1.8 | 1257704-57-6 |
| | nM, >40- and >800-fold versus JAK1 and TYK2. | |
| FG-4592 | FG-4592 is an HIF alpha prolyl hydroxylase inhibitor, | 808118-40-3 |
| | stabilizes HIF-2 and induces EPO production. Phase 2/3. | |
| CUDC-907 | CUDC-907 is a dual PI3K and HDAC inhibitor for PI3K | 1339928-25-4 |
| | and HDAC1/2/3/10 with IC50 of 19 nM and 1.7 nM/5 | |
| | nM/1.8 nM/2.8 nM, respectively. Phase 1. | |
| Olaparib (AZD2281, | Olaparib (AZD2281, KU0059436) is a selective inhibitor of | 763113-22-0 |
| Ku-0059436) | PARP1/2 with IC50 of 5 nM/1 nM, 300-times less effective | |
| | against tankyrase-1. Phase 1/2. | |
| IOX2 | IOX2 is a potent inhibitor of HIF-1alpha prolyl | 931398-72-0 |
| | hydroxylase-2 (PHD2) with IC50 of 21 nM, >100-fold | |
| | selectivity over JMJD2A, JMJD2C, JMJD2E, JMJD3, or | |
| | the 2OG oxygenase FIH. | |
| Veliparib (ABT-888) | Veliparib (ABT-888) is a potent inhibitor of PARP1 and PARP2 | 912444-00-9 |
| | with Ki of 5.2 nM and 2.9 nM, respectively. It is inactive to | |
| | SIRT2. Phase 1/2. | |
| AR-42 | AR-42 is an HDAC inhibitor with IC50 30 nM. | 935881-37-1 |
| Iniparib (BSI-201) | BSI-201 (Iniparib, SAR240550) is a PARP1 inhibitor with | 160003-66-7 |
| | demonstrated effectiveness in triple-negative breast cancer | |
| | (TNBC). Phase 3. | |
| PCI-24781 | PCI-24781 is a novel pan-HDAC inhibitor mostly targeting | 783355-60-2 |
| (Abexinostat) | HDAC1 with Ki of 7 nM, modest potent to HDACs 2, 3, 6, | |
| | and 10 and greater than 40-fold selectivity against HDAC8. | |
| | Phase 1/2. | |
| LAQ824 | LAQ824 (Dacinostat) is a novel HDAC inhibitor with | 404951-53-7 |
| (Dacinostat) | IC50 of 32 nM and can activate the p21 promoter. | |
| Quisinostat (JNJ- | JNJ-26481585 is a novel second-generation HDAC inhibitor | 875320-29-9 |
| 26481585) | with highest potency for HDAC1 with IC50 of 0.11 nM, modest | |
| | potent to HDACs 2, 4, 10, and 11; greater than 30-fold | |
| | selectivity against HDACs 3, 5, 8, and 9 and lowest potency to | |
| | HDACs 6 and 7. Phase 2. | |
| Rucaparib (AG- | Rucaparib (AG-014699, PF-01367338) is an inhibitor of | 459868-92-9 |
| 014699, PF- | PARP with Ki of 1.4 nM for PARP1, also showing binding | |
| 01367338) | affinity to eight other PARP domains. Phase 1/2. | |
| SRT1720 | SRT1720 is a selective SIRT1 activator with EC50 of 0.16 | 1001645-58-4 |
| | uM, but is >230-fold less potent for SIRT2 and SIRT3. | |
| CUDC-101 | CUDC-101 is a potent multi-targeted inhibitor against | 1012054-59-9 |
| | HDAC, EGFR and HER2 with IC50 of 4.4 nM, 2.4 nM, and | |
| | 15.7 nM, and inhibits class I/II HDACs, but not class III, Sir- | |
| | type HDACs. Phase 1. | |
| MC1568 | MC1568 is a selective HDAC inhibitor for maize HD1-A | 852475-26-4 |
| | with IC50 of 100 nM. It is 34-fold more selective for HD1-A | |
| | than HD1-B. | |
| Pracinostat (SB939) | SB939 is a potent pan-HDAC inhibitor with IC50 of 40-140 | 929016-96-6 |
| | nM with exception for HDAC6. It has no activity against the | |
| | class III isoenzyme SIRT I. Phase 2. | |
| Givinostat (ITF2357) | Givinostat (ITF2357) is a potent HDAC inhibitor for HDAC2, | 732302-99-7 |
| | HDAC1B and HDAC1A with IC50 of 10 nM, 7.5 nM and 16 | |
| | nM. Phase 1/2. | |
| AG-14361 | AG14361 is a potent inhibitor of PARP1 with Ki of <5 nM. It | 328543-09-5 |
| | is at least 1000-fold more potent than the benzamides. | |
| SGI-1776 free base | SGI-1776 is a novel ATP competitive inhibitor of Pim1 with | 1025065-69-3 |
| | IC50 of 7 nM, 50- and 10-fold selective versus Pim2 and | |
| | Pim3, also potent to Flt3 and haspin. Phase 1. | |
| Tubastatin A HCl | Tubastatin A is a potent and selective HDAC6 inhibitor | 1310693-92-5 |
| | with IC50 of 15 nM. It is selective (1000-fold more) | |
| | against all other isozymes except HDAC8 (57-fold more). | |
| PCI-34051 | PCI-34051 is a potent and specific HDAC8 inhibitor with | 950762-95-5 |
| | IC50 of 10 nM. It has greater than 200-fold selectivity over | |
| | HDAC1 and 6, more than 1000-fold selectivity over | |
| | HDAC2, 3, and 10. | |
| PFI-1 (PF-6405761) | PFI-1 is a selective BET (bromodomain-containing protein) | 1403764-72-6 |
| | inhibitor for BRD4 with IC50 of 0.22 uM. | |
| Sodium | Sodium Phenylbutyrate is a transcriptional regulators that act | 1716-12-7 |
| Phenylbutyrate | by altering chromatin structure via the modulation of HDAC | |
| | activity. | |
| AZD2461 | AZD2461 is a novel PARP inhibitor with low affinity | 1174043-16-3 |
| | for Pgp than Olaparib. Phase 1. | |
| Resminostat | Resminostat dose-dependently and selectively inhibits | 864814-88-0 |
| | HDAC1/3/6 with IC50 of 42.5 nM/50.1 nM/71.8 nM, less | |
| | potent to HDAC8 with IC50 of 877 nM. | |
| I-BET151 | I-BET151 (GSK1210151 A) is a novel selective BET | 1300031-49-5 |
| (GSK1210151A) | inhibitor for BRD2, BRD3 and BRD4 with IC50 of 0.5 uM, | |
| | 0.25 uM, and 0.79 uM, respectively. | |
| AZD1480 | AZD1480 is a novel ATP-competitive JAK2 inhibitor with | 935666-88-9 |
| | IC50 of 0.26 nM, selectivity against JAK3 and Tyk2, and to a | |
| | smaller extent against JAK1. Phase 1. | |
| XL019 | XL019 is a potent and selective JAK2 inhibitor with IC50 of | 945755-56-6 |
| | 2.2 nM, exhibiting >50-fold selectivity over JAK1, JAK3 and | |
| | TYK2. Phase 1. | |
| Tubacin | Tubacin is a highly potent and selective, reversible, cell- | 537049-40-4 |
| | permeable HDAC6 inhibitor with an IC50 of 4 nM, | |
| | approximately 350-fold selectivity over HDAC1. | |
| ZM 39923 HCl | ZM 39923 is an JAK1/3 inhibitor with pIC50 of 4.4/7.1, | 1021868-92-7 |
| | almost no activity to JAK2 and modestly potent to | |
| | EGFR; also found to be sensitive to transglutaminase. | |
| 3-Deazaneplanocin | 3-deazaneplanocin A (DZNeP), an analog of adenosine, is | 120964-45-6 |
| A (DZNeP) | a competitive inhibitor of S-adenosylhomocysteine | |
| | hydrolase with Ki of 50 pM. | |
| SMI-4a | SMI-4a is a potent inhibitor of Pim1 with IC50 of 17 | 438190-29-5 |
| | nM, modest potent to Pim-2, does not significantly | |
| | inhibit other serine/threonine- or tyrosine-kinases. | |
| (+)-JQ1 | (+)-JQ1 is a BET bromodomain inhibitor, with IC50 of 77 | 1268524-70-4 |
| | nM/33 nM for BRD4(1/2), binding to all bromodomains of | |
| | the BET family, but not to bromodomains outside the BET | |
| | family. | |
| BMN 673 | BMN 673 is a novel PARP inhibitor with IC50 of 0.58 nM. It | 1207456-01-6 |
| | is also a potent inhibitor of PARP-2, but does not inhibit PARG | |
| | and is highly sensitive to PTEN mutation. Phase 1. | |
| Pacritinib (SB1518) | Pacritinib (SB1518) is a potent and selective inhibitor of | 937272-79-2 |
| | Janus Kinase 2 (JAK2) and Fms-Like Tyrosine Kinase-3 | |
| | (FLT3) with IC50s of 23 and 22 nM, respectively. | |
| Rocilinostat (ACY- | Rocilinostat (ACY-1215) is a selective HDAC6 inhibitor with | 1316214-52-4 |
| 1215) | IC50 of 5 nM. It is >10-fold more selective for HDAC6 than | |
| | HDAC1/2/3 (class I HDACs) with slight activity against | |
| | HDAC8, minimal activity against HDAC4/5/7/9/11, Sirtuin1, | |
| | [symbol] | |
| UPF 1069 | UPF 1069 is a selective PARP2 inhibitor with IC50 of 0.3 nM. | 1048371-03-4 |
| | It is ~27-fold selective against PARP1. | |
| EPZ5676 | EPZ-5676 is an S-adenosyl methionine (SAM) competitive | 1380288-87-8 |
| | inhibitor of protein methyltransferase DOT1L with Ki of 80 | |
| | pM, demonstrating >37,000-fold selectivity against all other | |
| | PMTs tested, inhibits H3K79 methylation in tumor. Phase 1. | |
| GSK J4 HCl | GSK J4 HCl is a cell permeable prodrug of GSK J1, which is | 1797983-09-5 |
| | the first selective inhibitor of the H3K27 histone demethylase | |
| | JMJD3 and UTX with IC50 of 60 nM and inactive against a | |
| | panel of demethylases of the JMJ family. | |
| EPZ004777 | EPZ004777 is a potent, selective DOT1L inhibitor with | 1338466-77-5 |
| | IC50 of 0.4 nM. | |
| Bromosporine | Bromosporine is a broad spectrum inhibitor for bromodomains | 1619994-69-2 |
| | with IC50 of 0.41 uM, 0.29 uM, 0.122 uM and 0.017 uM for | |
| | BRD2, BRD4, BRD9 and CECR2, respectively. | |
| Lomeguatrib | Lomeguatrib is a potent inhibitor of O6-alkylguanine-DNA- | 192441-08-0 |
| | alkyltransferase with IC50 of 5 nM. | |
| I-BET-762 | I-BET-762 is an inhibitor for BET proteins with IC50 of ~35 | 1260907-17-2 |
| | nM, suppresses the production of proinflammatory | |
| | proteins by macrophages and blocks acute inflammation, | |
| | highly selective over other bromodomain-containing | |
| | proteins. | |
| RGFP966 | RGFP966 is an HDAC3 inhibitor with IC50 of 0.08 uM, | 1396841-57-8 |
| | exhibits >200-fold selectivity over other HDAC. | |
| SGC 0946 | SGC 0946 is a highly potent and selective DOT1L | 1561178-17-3 |
| | methyltransferase inhibitor with IC50 of 0.3 nM, is inactive | |
| | against a panel of 12 PMTs and DNMT1. | |
| SGI-1027 | SGI-1027 is a DNMT inhibitor with IC50 of 6, 8, 7.5 uM | 1020149-73-8 |
| | for DNMT1, DNMT3A, and DNMT3B. | |
| EPZ-6438 | EPZ-6438 is a potent, and selective EZH2 inhibitor with Ki | 1403254-99-8 |
| | and IC50 of 2.5 nM and 11 nM, exhibiting a 35-fold | |
| | selectivity versus EZH1 and >4,500-fold selectivity relative | |
| | to 14 other HMTs. | |
| RVX-208 | RVX-208 is a potent BET bromodomain inhibitor with | 1044870-39-4 |
| | IC50 of 0.510 uM for BD2, about 170-fold selectivity over | |
| | BD1. Phase 2. | |
| MM-102 | MM-102 is a high-affinity peptidomimetic MLL1 inhibitor | 1417329-24-8 |
| | with IC50 of 0.4 uM. | |
| RG2833 (RGFP109) | RG2833 (RGFP109) is a brain-penetrant HDAC inhibitor | 1215493-56-3 |
| | with IC50 of 60 nM and 50 nM for HDAC1 and HDAC3, | |
| | respectively. | |
| SGC-CBP30 | SGC-CBP30 is a potent CREBBP/EP300 inhibitor with | 1613695-14-9 |
| | IC50 of 21 nM and 38 nM, respectively. | |
| ME0328 | ME0328 is a potent and selective PARP inhibitor with | 1445251-22-8 |
| | IC50 of 0.89 uM for PARP3, about 7-fold selectivity over | |
| | PARP1. | |
| UNC669 | UNC669 is a potent and selective MBT (malignant brain | 1314241-44-5 |
| | tumor) inhibitor with IC50 of 6 uM for L3MBTL1, 5- and | |
| | 11-fold selective over L3MBTL3 and L3MBTL4. | |
| OTX015 | OTX015 is a potent BET bromodomain inhibitor with | 202590-98-5 |
| | EC50 ranging from 10 to 19 nM for BRD2, BRD3, and | |
| | BRD4. Phase 1. | |
| Nexturastat A | Nexturastat A is a potent and selective HDAC6 inhibitor | 1403783-31-2 |
| | with IC50 of 5 nM, >190-fold selectivity over other | |
| | HDACs. | |
| OG-L002 | OG-L002 is a potent and specific LSD1 inhibitor with IC50 | 1357302-64-7 |
| | of 20 nM, exhibiting 36- and 69-fold selectivity over | |
| | MAO-B and MAO-A, respectively. | |
| C646 | C646 is an inhibitor for histone acetyltransferase, and inhibits | 328968-36-1 |
| | p300 with a Ki of 400 nM. Preferentially selective for p300 | |
| | versus other acetyltransferases. | |
| UNC1215 | UNC1215 is a potent and selective MBT (malignant brain | 1415800-43-9 |
| | tumor) antagonist, which binds L3MBTL3 with IC50 of 40 | |
| | nM and Kd of 120 nM, 50-fold selective versus other | |
| | members of the human MBT family. | |
| IOX1 | IOX1 is a potent and broad-spectrum inhibitor of 2OG | 5852-78-8 |
| | oxygenases, including the JmjC demethylases. | |
| AZD1208 | AZD1208 is a potent, and orally available Pirn kinase | 1204144-28-4 |
| | inhibitor with IC50 of 0.4 nM, 5 nM, and 1.9 nM for | |
| | Pim1, Pim2, and Pim3, respectively. Phase 1. | |
| CX-6258 HCl | CX-6258 HCl is a potent, orally efficacious pan-Pim | 1353859-00-3 |
| | kinase inhibitor with IC50 of 5 nM, 25 nM and 16 nM for | |
| | Pim1, Pim2, and Pim3, respectively. | |
| CPI-203 | CPI-203 is a potent BET bromodomain inhibitor with IC50 | 1446144-04-2 |
| | of 37 nM for BRD4. | |
| TMP269 | TMP269 is a potent, selective class IIa HDAC inhibitor with | 1314890-29-3 |
| | IC50 of 157 nM, 97 nM, 43 nM and 23 nM for HDAC4, | |
| | HDAC5, HDAC7 and HDAC9, respectively. | |
| Filgotinib | Filgotinib (GLPG0634) is a selective JAK1 inhibitor with | 1206161-97-8 |
| (GLPG0634) | IC50 of 10 nM, 28 nM, 810 nM, and 116 nM for JAK1, | |
| | JAK2, JAK3, and TYK2, respectively. Phase 2. | |
| Isoliquiritigenin | A flavonoid found in licorice root that displays antioxidant, | 961-29-5 |
| | anti-inflammatory, and antitumor activities; induces quinone | |
| | reductase-1 with a concentration required to double activity of | |
| | 1.8 μM in mouse hepatoma cells. | |
| Ellagic Acid | A polyphenolic antioxidant that is abundant in many fruits, | 476-66-4 |
| | vegetables, plant bark, and peels; has anti-carcinogenic, anti- | |
| | mutagenic, anti-inflammatory, and organ-preserving | |
| | properties; blocks methylation of H3R17 by CARM1 without | |
| | significantly altering histone acetylase or DNA | |
| | methyltransferase activity. | |
| Sodium Butyrate | A short chain fatty acid that inhibits HDACs, induces growth | 156-54-7 |
| | arrest, differentiation and apoptosis in cancer cells, and | |
| | suppresses inflammation by reducing the expression of pro- | |
| | inflammatory cytokines. | |
| Etoposide | An inhibitor of topoisomerase II (IC50 = 60.3 μM); can have | 33419-42-0 |
| | much greater potencies when evaluated in cell-based | |
| | cytotoxicity assays (e.g., IC50 = 5.14 nM for MCF-7 cells); | |
| | can also inhibit nuclear receptor coactivator 3 (IC50 of 2.48 | |
| | μM). | |
| Tenovin-1 | A small molecule activator of p53 that decreases the | 380315-80-0 |
| | growth of BL2 Burkitt's lymphoma and ARN8 melanoma | |
| | cells; inhibits the deacetylase activity of purified human | |
| | SIRT1 and SIRT2. | |
| Gemcitabine | A nucleoside analog that arrests tumor growth and induces | 95058-81-4 |
| | apoptosis by inhibiting DNA replication and repair; inhibits | |
| | repair-mediated DNA demethylation inducing epigenetic | |
| | gene silencing and has broad antiretroviral activity. | |
| CPTH2 | Specifically inhibits Gcn5-dependent acetylation of histone | 357649-93-5 |
| (hydrochloride) | H3K14 at a concentration of 0.8 mM both in vitro and in vivo. | |
| UNC0638 | A potent, selective G9a and GLP HMTase inhibitor (IC50s = <15 | 1255580-76-7 |
| | and 19 nM, respectively); inhibits H3K9 dimethylation in | |
| | MDA-MB231 cells (IC50 = 81 nM) and demonstrates | |
| | favorable separation of functional and toxic effects. | |
| Phthalazinone | A potent inhibitor of Aurora A kinase (IC50 = 31 nM); does not | 88048-62-7 |
| pyrazole | inhibit Aurora B kinase at doses up to 100 μM; inhibits the | |
| | proliferation of HCT116, Colo205, and MCF-7 cells (IC50 = | |
| | 7.8, 2.9, and 1.6 μM, respectively). | |
| 4-iodo-SAHA | A hydrophobic derivative of the class I and class II HDAC | 1219807-87-0 |
| | inhibitor SAHA that demonstrates >60% inhibition of | |
| | HDAC1 and HDAC6 activity in a deacetylase activity assay; | |
| | inhibits proliferation of SK-BR-3 breast-derived, HT29 | |
| | colon-derived, and U937 leukemia cell lines with EC50 | |
| | values of 1.1, 0.95, and 0.12 μM, respectively. | |
| UNC0321 | A potent and selective G9a HMTase inhibitor (IC50 = 6 | 1238673-32-9 |
| (trifluoroacetate | nM; Ki = 63 pM); more than 40,000-fold selective for G9a | |
| salt) | over SET7/9, SET8, PRMT3, and JMJD2E. | |
| (−)-Neplanocin A | Potently and irreversibly inactivates SAH hydrolase (Ki = | 72877-50-0 |
| | 8.39 nM); has antitumor activity against mouse leukemia | |
| | L1210 cells and broad-spectrum antiviral activity. | |
| Cl-Amidine | An inhibitor of PAD4 deimination activity (IC50 = 5.9 μM) | 913723-61-2 |
| (trifluoroacetate) | that also inhibits PAD1 and PAD3 (IC50 = 0.8 and 6.2 μM, | |
| | respectively); dose dependently decreases the citrulline | |
| | content in serum and joints and reduces the development of | |
| | IgG autoantibodies in a CIA mouse model of inflammatory | |
| | arthritis. | |
| F-Amidine | Inhibits PAD4 activity (IC50 = 21.6 μM) as well as PAD1 | 877617-46-4 |
| (trifluoroacetate | and PAD3 activity (IC50s = 29.5 and 350 μM, | |
| salt) | respectively); cytotoxic to HL-60, MCF-7, and HT-29 | |
| | cancer cell lines (IC50s = 0.5, 0.5 and 1 μM, respectively). | |
| JGB1741 | A SIRT1-specific inhibitor (IC50 = 15 μM); inhibits | 1256375-38-8 |
| | metastatic breast cancer MDA-MB 231 cell proliferation | |
| | (IC50 = 512 nM), dose-dependently increasing p53 | |
| | acetylation and p53-mediated apoptosis in these cells. | |
| CCG-100602 | Inhibits RhoA/C-mediated, SRF-driven luciferase | 1207113-88-9 |
| | expression in PCS prostate cancer cells with an IC50 value | |
| | of 9.8 μM. | |
| CAY10669 | An inhibitor of the HAT PCAF (p300/CREB-binding | 1243583-88-1 |
| | protein-associated factor; IC50 = 662 μM), displaying a 2- | |
| | fold improvement in inhibitory potency over anacardic acid; | |
| | dose dependently inhibits histone H4 acetylation in HepG2 | |
| | cells in vitro at 30-60 μM. | |
| Delphinidin | A natural plant pigment which induces the release of nitric | 528-53-0 |
| (chloride) | oxide by vascular endothelium, causing vasorelaxation; | |
| | inhibits signaling through EGFRs, suppressing the | |
| | expression of ERa and inducing both apoptosis and | |
| | autophagy at a dose of 1-40 μM; inhibits the HAT activities | |
| | of p300/CBP (IC50 = ~30 μM). | |
| MI-2 | Potently binds menin, blocks the menin-MLL fusion | 1271738-62-5 |
| (hydrochloride) | protein interaction (IC50 = 0.45 μM), and induces | |
| | apoptosis in cells expressing MLL fusion proteins. | |
| MI-nc | A weak inhibitor of the menin-MLL fusion protein | 1359873-45-2 |
| (hydrochloride) | interaction (IC50 = 193 μM), intended as a negative control | |
| | compound for tests involving MI-2. | |
| Octyl-.alpha.- | A stable, cell-permeable form of a-ketoglutarate which | 876150-14-0 |
| ketoglutarate | accumulates rapidly and preferentially in cells with a | |
| | dysfunctional TCA cycle; stimulates PHD activity and | |
| | increases HIF-1a turnover when used at 1 mM; | |
| | competitively blocks succinate- or fumarate-mediated | |
| | inhibition of PHD. | |
| Daminozide | A selective inhibitor of the human 2-oxoglutarate (JmjC) | 1596-84-5 |
| | histone demethylases KDM2A, PHF8, and KDM7A (IC50s = | |
| | 1.5, 0.55, and 2.1 μM, respectively). | |
| GSK-J1 (sodium | A potent, cell impermeable inhibitor of the H3K27 histone | 1373422-53-7 |
| salt) | demethylases JMJD3 and UTX (IC50s = 18 and 56 μM, | |
| | respectively as measured by mass spectrometry; IC50 = 60 | |
| | nM in JMJD3 antibody-based assays). | |
| GSK-J2 (sodium | A pyridine regio-isomer of GSK-J1 which poorly inhibits | 1394854-52-4 |
| salt) | JMJD3 (IC50 > 100 μM), making it an appropriate negative | |
| | control for in vitro studies involving GSK-J1. | |
| GSK-J5 | A pyridine regio-isomer of the JMJD3 inhibitor GSK-J4; | 1797983-32-4 |
| (hydrochloride) | cell-permeable and hydrolyzed to a free base, which is a | |
| | weak inhibitor of JMJD3 (IC50 > 100 μM), making it an | |
| | ideal negative control molecule. | |
| HC Toxin | A cell-permeable, reversible inhibitor of HDACs (IC50 = 30 | 83209-65-8 |
| | nM). | |
| (+)-Abscisic Acid | A plant hormone with diverse roles in disease resistance, | 21293-29-8 |
| | plant development, and response to stresses; regulates gene | |
| | expression and may contribute to epigenetic changes at the | |
| | chromatin level. | |
| 4-pentynoyl- | An acyl-CoA donor that can be metabolically transferred | 50347-32-5 |
| Coenzyme A | onto lysine residues of proteins by lysine acetyltransferases; | |
| (trifluoroacetate | an azide-alkyne bioconjugation reaction, known as click | |
| salt) | chemistry, can then be used to tag the acetylated proteins | |
| | with fluorescent or biotinylated labels for subsequent | |
| | analysis. | |
| coumarin-SAHA | A fluorescent probe that competitively binds HDAC; | 1260635-77-5 |
| | demonstrates fluorescence excitation and emission maxima | |
| | of 325 and 400 nm, respectively, which is quenched by | |
| | 50% when bound to HDAC. | |
| SAHA-BPyne | A SAHA derivative with a benzophenone crosslinker and | 930772-88-6 |
| | an alkyne tag to be used for profiling HDAC activities in | |
| | proteomes and live cells; labels HDAC complex proteins | |
| | both in proteomes at 100 nM and in live cells at 500 nM; | |
| | IC50 = ~3 μM for inhibition of HDAC activity in HeLa cell | |
| | nuclear lysates in an HDAC activity assay. | |
| UNC0631 | A potent and selective inhibitor of G9a activity in vitro | 1320288-19-4 |
| | (IC50 = 4 nM) and G9a/GLP-mediated dimethylation of | |
| | histone 3 on lysine 9 in MDA-MB-231 cells (IC50 = 25 | |
| | nM). | |
| UNC0646 | A potent and selective inhibitor of G9a and GLP activities | 1320288-17-2 |
| | in vitro (IC50s = 6 and 15 nM, respectively) and G9a/GLP- | |
| | mediated dimethylation of histone 3 on lysine 9 in MDA- | |
| | MB-231 cells (IC50 = 26 nM). | |
| GSK4112 | A synthetic agonist for REV-ERBa (EC50 = 0.4 μM) that | 1216744-19-2 |
| | mimics the action of heme; at 10 μM inhibits the | |
| | expression of the circadian target gene bmal1 and reduces | |
| | glucose output by 30% in mouse primary hepatocytes by | |
| | repressing the expression of several gluconeogenic genes. | |
| Lestaurtinib | A staurosporine analog that potently inhibits JAK2 kinase | 111358-88-4 |
| | (IC50 = 1 nM) and downstream targets STAT5 (IC50 = 10- | |
| | 30 nM) and STAT3 in a human erythroleukemic cell line | |
| | expressing the JAK2V617F mutation; potently inhibits the | |
| | epigenetic kinase PRK1 (PKN1) in vitro (IC50 = 8.6 nM). | |
| Tenovin-6 | A analog of tenovin-1; elevates p53 activity in MCF-7 cells | 1011557-82-6 |
| | at 10 μM and reduces growth of ARN8 melanoma | |
| | xenograft tumors in SCID mice at a dose of 50 mg/kg. | |
| Chaetocin | A fungal mycotoxin that inhibits the Lys9-specific histone | 28097-03-2 |
| | methyltransferases SU(VAR)3-9 (IC50 = 0.8 μM), G9a | |
| | (IC50 = 2.5 μM), and DIM5 (IC50 = 3 μM). | |
| CBHA | HDAC1 and HDAC3 inhibitor (ID50 = 0.01 and 0.07 μM, | 174664-65-4 |
| | respectively, in vitro); induces apoptosis in nine different | |
| | neuroblastoma cell lines in culture (0.5-4.0 μM) and | |
| | completely suppresses neuroblastoma tumor growth in SCID | |
| | mice at 200 mg/kg. | |
| Mirin | An inhibitor of the DNA damage sensor MRN, inhibiting | 299953-00-7 |
| | MRN-dependent phosphorylation of histone H2AX (IC50 = | |
| | 66 μM); prevents activation of ATM by blocking the | |
| | nuclease activity of Mre11; induces G2 arrest, abolishes the | |
| | radiation-induced G2/M checkpoint, and prevents | |
| | homology-directed repair of DNA damage. | |
| 6-Thioguanine | A thio analog of the purine base guanine that incorporates | 154-42-7 |
| | into DNA during replication, inducing double-strand breaks | |
| | that destabilize its structure and result in cytotoxicity; used | |
| | as a chemotherapeutic for acute leukemia and other types of | |
| | cancer, including BRCA2-mutated tumors. | |
| SIRT1/2 Inhibitor IV | A cell-permeable inhibitor of SIRT1 (IC50 = 56 μM) and | 14513-15-6 |
| | SIRT2 (IC50 = 59 μM); less effectively inhibits SIRT5 | |
| | (IC50 > 300 μM) and has no effect on class I and II HDACs; | |
| | sensitizes H460 lung cancer cells to etoposide and | |
| | paclitaxel; blocks a SIRT1-dependent hypoxic response in | |
| | vivo. | |
| CAY10591 | An activator of SIRT1 that decreases TNF-a levels from 325 | 839699-72-8 |
| | pg/ml (control) to 104 and 53 pg/ml at 20 and 60 μM, | |
| | respectively; exhibits a significant dose-dependent effect on | |
| | fat mobilization in differentiated adipocytes. | |
| S- | An amino acid derivative and an intermediate, by-product, or | 979-92-0 |
| Adenosylhomocysteine | modulator of several metabolic pathways, including the | |
| | activated methyl cycle and cysteine biosynthesis; also a | |
| | product of SAM-dependent methylation of biological | |
| | molecules, including DNA, RNA, and histones, and other | |
| | proteins. | |
| HNHA | A cell-permeable inhibitor of HDAC activity (IC50 = 100 | 926908-04-5 |
| | nM). | |
| 2-Hydroxyglutaric | An a-hydroxy acid, overproduced in 2-hydroxyglutaric | 40951-21-1 |
| Acid (sodium salt) | aciduria; mutations in IDH1 and IDH2 cause these | |
| | enzymes to convert isocitrate to 2-hydroxyglutarate; | |
| | competitively inhibits a-ketoglutarate-dependent | |
| | dioxygenases, including lysine demethylases and DNA | |
| | hydroxylases. | |
| 3,3′- | Phytochemical from cruciferous vegetables that | 5/4/1968 |
| Diindolylmethane | demonstrates anticancer and chemopreventative effects (10- | |
| | 30 μM) involving the induction of Phase 2 enzymes, | |
| | promotion of apoptosis, induction of cell cycle arrest, | |
| | inhibition of cell proliferation, and inhibition of histone | |
| | deacetylases and DNA methylation activities. | |
| S-(5′-Adenosyl)-L- | A ubiquitous methyl donor involved in a wide variety of | 86867-01-8 |
| methionine chloride | biological reactions, including those mediated by DNA | |
| (hydrochloride) | and protein methyltransferases | |
| | A stable salt of SAM that is included in nutritional | |
| | supplements for oral use; reportedly ameliorates | |
| | depression, pain associated with osteoarthritis and | |
| | fibromyalgia, and liver toxicity. | |
| Pimelic | A slow, tight-binding inhibitor of class I HDACs, | 937039-45-7 |
| Diphenylamide 106 | progressively binding HDACs and remaining bound after | |
| | wash-out; inhibits class I HDACs (IC50 = 150, 760, 370, and | |
| | 5,000 nM for HDAC1, 2, 3, and 8, respectively) but not class | |
| | II HDACs (IC50 > 180 μM for HDAC4, 5, and 7). | |
| 2′,3′,5′-triacetyl-5- | A prodrug form of 5-azacytidine, an inhibitor of | 10302-78-0 |
| Azacytidine | DNA methyltransferaes, that may reverse | |
| | epigenetic changes. | |
| UNC0224 | A potent and selective G9a HMTase inhibitor (IC50 = 15 | 1197196-48-7 |
| | nM, Kd = 23 mM); more than 1,000-fold selective for G9a | |
| | over SET7/9 and SET8. | |
| Sinefungin | A nucleoside structurally related to SAH and SAM that | 58944-73-3 |
| | inhibits SET domain-containing methyltransferases (IC50 | |
| | values range from 0.120 μM). | |
| Pyroxamide | An inhibitor of HDAC, including HDAC1 (IC50 = 0.1-0.2 | 382180-17-8 |
| | μM); induces growth suppression and cell death of certain | |
| | types of cancer cells in culture. | |
| WDR5-0103 | A small molecule that binds a peptide-binding pocket on | 890190-22-4 |
| | WDR5 (Kd = 450 nM), inhibiting the catalytic activity of the | |
| | MLL core complex in vitro (IC50 = 39 μM). | |
| AMI-1 (sodium salt) | A cell permeable inhibitor of PRMTs; inhibits both yeast | 20324-87-2 |
| | Hmt1p and human PRMT1 (IC50 = 3.0 and 8.8 μM, | |
| | respectively); also effectively blocks the activity of PRMTs | |
| | 3, 4, and 6 but not that of lysine methyltransferases; inhibits | |
| | HIV-1 reverse transcriptase (IC50 = 5.0 μM). | |
| GSK343 | A selective, cell-permeable EZH2 inhibitor (IC50 = 4 nM) | 1346704-33-3 |
| | that has been shown to inhibit the trimethylation of H3K27 | |
| | in HCC1806 cells with an IC50 value of 174 nM. | |
| I-CBP112 | A selective inhibitor of CBP and EP300 which directly | 1640282-31-0 |
| (hydrochloride) | binds their bromodomains (Kds = 0.142 and 0.625 μM); | |
| | shows only weak cross reactivity with the bromodomains of | |
| | BET proteins and shows no interaction with other | |
| | bromodomains. | |
| UNC1999 | A selective, cell-permeable EZH2 inhibitor (IC50 = 2 | 1431612-23-5 |
| | nM) that has been shown to inhibit H3K27methylation in | |
| | MCF10A cells with an IC50 value of 124 nM. | |
| PFI-3 | Binds avidly and selectively to the structurally-similar | 1819363-80-8 |
| | bromodomains of SMARCA4 and PB1 (domain 5) with Kd | |
| | values of 89 and 48 nM, respectively; also interacts with the | |
| | bromodomain of SMARCA2; does not interact with other | |
| | bromodomains or with a panel of kinases. | |
| 2,4-DPD | A cell permeable, competitive inhibitor of HIF-PH with | 41438-38-4 |
| | effective concentrations in the low μM range. | |
| DMOG | A cell permeable, competitive inhibitor of HIF-1a prolyl | 89464-63-1 |
| | hydroxylase; stabilizes HIF-1a expression at normal oxygen | |
| | tensions in cultured cells at concentrations between 0.1 and | |
| | 1 mM. | |
| CAY10398 | An inhibitor of HDAC (IC50 = 10 μM) | 193551-00-7 |
| RSC-133 | Promotes the reprogramming of human somatic cells to | 1418131-46-0 |
| | pluripotent stem cells; increases the number of human | |
| | foreskin fibroblasts that express alkaline phosphatase when | |
| | used at 10 μM with four standard reprogramming factors; | |
| | down-regulates inducers of cellular senescence and inhibits | |
| | Dnmt1 and HDAC1. | |
| N-Oxalylglycine | A cell permeable inhibitor of a-ketoglutarate-dependent | 5262-39-5 |
| | enzymes, including JMJD2A, JMJD2C, and JMJD2E (IC50s = | |
| | 250, 500, and 24 μM, respectively); inhibits the prolyl | |
| | hydroxylase domain-containing proteins PHD1 and PHD2 | |
| | with IC50 values of 2.1 and 5.6 μM, respectively. | |
| Chidamide | An HDAC inhibitor that increases histone H3 acetylation | 743420-02-2 |
| | levels in LoVo and HT-29 colon cancer cells at | |
| | concentrations as low as 4 μM; dose-dependently decreases | |
| | the activation of several oncogenic signaling kinases and | |
| | induces cell cycle arrest in colon cancer cells. | |
| EPZ005687 | A potent, selective inhibitor of the lysine methyltranferase | 1396772-26-1 |
| | EZH2 (Ki = 24 nM), the enzymatic subunit of PRC2; blocks | |
| | trimethylation of the PRC2 target H3K27 (IC50 = 80 nM), | |
| | decreasing the proliferation of lymphoma cells carrying | |
| | mutant, but not wild-type, EZH2. | |
| AK-7 | A cell- and brain-permeable inhibitor of SIRT2 (IC50 = 15.5 | 420831-40-9 |
| | μM); dimishes neuronal cell death induced by mutant | |
| | huntingtin fragment in culture; down-regulates cholesterol | |
| | biosynthetic gene expression and reduces total cholesterol | |
| | levels in neurons in vivo. | |
| UNC0642 | A selective inhibitor of G9a and GLP methyltransferases that | 1481677-78-4 |
| | competitively inhibits binding of H3K9 substrates with a Ki = | |
| | 3.7 nM; reduces H3K9 dimethylation levels in MDA-MB- | |
| | 231 and PANC-1 cells (IC50s = 110 and 40 nM, | |
| | respectively); displays improved pharmacokinetic properties | |
| | relative to UNC0638. | |
| (R)-PFI-2 | A potent, cell-permeable inhibitor of SET7/9 (IC50 = 2 | 1627607-87-7 |
| (hydrochloride) | nM) that demonstrates greater than 1,000-fold selectivity | |
| | over a panel of 18 other methyltransferases. | |
| HPOB | A potent, selective inhibitor of HDAC6 (IC50 = 56 nM); | 1429651-50-2 |
| | induces acetylation of a-tubulin but not histones; enhances | |
| | the cytotoxicity of the broad spectrum HDAC inhibitor | |
| | SAHA against cancer cells in nude mice carrying an | |
| | androgen-dependent CWR22 human prostate cancer | |
| | xenograft. | |
| 2-hexyl-4-Pentynoic | Inhibits HDAC activity much more potently (IC50 = 13 μM) | 96017-59-3 |
| Acid | than valproic acid (IC50 = 398 μM); induces histone | |
| | hyperacetylation in cerebellar granule cells significantly at 5 | |
| | μM; induces the expression of Hsp70-1a and Hsp70-1b and | |
| | protects cerebellar granule cells from glutamate-induced | |
| | excitotoxicity. | |
| JIB-04 | A pyridine hydrazone that broadly inhibits Jumonji histone | 199596-05-9 |
| | demethylases (IC50 values are 230, 340, 435, 445, 855, and | |
| | 1100 nM for JARID1A, JMJD2E, JMJD2B, JMJD2A, | |
| | JMJD3 and JMJD2C, respectively); inhibits Jumonji | |
| | demethylase activity, alters gene expression, and blocks | |
| | viability of cancer cells both in vitro and in vivo. | |
| CAY10683 | A potent HDAC inhibitor that inhibits HDAC2 and HDAC6 | 1477949-42-0 |
| | with IC50 values of 0.119 and 434 nM; ineffective against | |
| | HDAC4 (IC50 = >1,000 nM); inhibits the growth of HCT- | |
| | 116 cells and HuT-78 cells (GI50 = 29.4 and 1.4 μM, | |
| | respectively) more effectively than human dermal | |
| | fibroblasts (GI50 = >100 μM). | |
| GSK 126 | A selective, SAM-competitive small molecule inhibitor of | 1346574-57-9 |
| | EZH2 methyltransferase activity (Ki = 0.57 nM; IC50 = 9.9 | |
| | nM versus that of EZH1: Ki = 89 nM; IC50 = 680 nM); | |
| | inhibits global H3K27me3 levels, inhibiting the | |
| | proliferation of EZH2 mutant DLBCL cell lines (IC50 = | |
| | 28-61 nM) as well as the growth of EZH2 mutant DLBCL | |
| | xenografts in mice receiving a daily dose of 50 mg/kg. | |
| MS-436 | A potent BRD4 bromodomain inhibitor that binds BD1 | 1395084-25-9 |
| | more avidly than BD2 (Ki values are 30-50 nM for BD1 and | |
| | 340 nM for BD2); also binds BD1 and BD2 of BRD3 (Kis = | |
| | 100 and 140 nM, respectively) as well as bromodomains of | |
| | other BET and non-BET proteins with low micromolar | |
| | affinities. | |
| 5-Methylcytidine | A modified nucleoside derived from 5-methylcytosine and is | 2140-61-6 |
| | a minor constituent of RNA as well as DNA for certain | |
| | organisms; used in epigenetics research, especially in studies | |
| | involving DNA methylation processes. | |
| AGK7 | An inactive control to be used in experiments with AGK2; | 304896-21-7 |
| | has IC50 values greater than 50 μM on SIRT1 and SIRT2 | |
| | and greater than 5 μM on SIRT3. | |
| 5-Methyl-2′- | A pyrimidine nucleoside used in epigenetics research to | 838-07-3 |
| deoxycytidine | investigate the dynamics of DNA methylation pattern in the | |
| | control of gene expression. | |
| B32B3 | A cell-permeable, ATP-competitive inhibitor of VprBP that | 294193-86-5 |
| | blocks phosphorylation of histone 2A at Thr120 in DU-145 | |
| | human prostate cancer cells (IC50 = 500 nM); strongly | |
| | suppresses the proliferation of DU-145 cells, which | |
| | overexpress VprBP, both in vitro and in xenograft tumors in | |
| | mice. | |
| GSK-LSD1 | An irreversible, mechanism-based inhibitor of LSD1 (IC50 = | 1431368-48-7 |
| (hydrochloride) | 16 nM); induces gene expression changes in various | |
| | cancer cell lines, inhibiting their proliferation (EC50s < | |
| | 5 nM). | |
| AZ 505 | A potent inhibitor of SMYD2 (IC50 = 0.12 μM) that is | 1035227-43-0 |
| | without effect on a panel of other protein lysine | |
| | methyltransferases. | |
| BRD73954 | A small molecule inhibitor that potently and selectively | 1440209-96-0 |
| | inhibits both HDAC6 and HDAC8 (IC50s = 36 and 120 nM, | |
| | respectively). | |
| CPI-360 | CPI-360 is a potent, selective, and SAM-competitive EZH1 | 1802175-06-9 |
| | inhibitor with IC50 of 102.3 nM, >100-fold selectivity | |
| | over other methyltransferases. | |
| Remodelin | Remodelin is a potent acetyl-transferase NAT10 inhibitor. | 1622921-15-6 |
| UNC0379 | UNC0379 is a selective, substrate competitive inhibitor of | 1620401-82-2 |
| | N-lysine methyltransferase SETD8 with IC50 of 7.9 μM, | |
| | high selectivity over 15 other methyltransferases. | |
| GSK2801 | GSK2801 is a selective bromodomains BAZ2A/B inhibitor | 1619994-68-1 |
| | with KD of 257 nM and 136 nM, respectively. | |
| CPI-169 | CPI-169 is a potent, and selective EZH2 inhibitor with | 1450655-76-1 |
| | IC50 of 0.24 nM, 0.51 nM, and 6.1 nM for EZH2 WT, | |
| | EZH2 Y641N, and EZH1, respectively. | |
| ORY-1001 (RG- | ORY-1001 (RG-6016) is an orally active and selective | 1431326-61-2 |
| 6016) | lysine-specific demethylase LSD1/KDM1A inhibitor with | |
| | IC50 of <20 nM, with high selectivity against related FAD | |
| | dependent aminoxidases. Phase 1. | |
| SP2509 | SP2509 is a selective histone demethylase LSD1 inhibitor | 1423715-09-6 |
| | with IC50 of 13nM, showing no activity against MAO-A, | |
| | MAO-B, lactate dehydrogenase and glucose oxidase. | |
| EI1 | EI1 is a potent and selective EZH2 inhibitor with IC50 of | 1418308-27-6 |
| | 15 nM and 13 nM for EZH2 (WT) and EZH2 (Y641F), | |
| | respectively. | |
| BRD4770 | BRD4770 is a histone methyltransferase G9a inhibitor with | 1374601-40-7 |
| | IC50 of 6.3 μM, and induces cell senescence. | |
| GSK503 | GSK503 is a potent and specific EZH2 | 1346572-63-1 |
| | methyltransferase inhibitor. | |
| GSK1324726A (I- | GSK1324726A (I-BET726) is a highly selective inhibitor of | 1300031-52-0 |
| BET726) | BET family proteins with IC50 of 41 nM, 31 nM, and 22 nM | |
| | for BRD2, BRD3, and BRD4, respectively. | |
| MI-3 (Menin-MLL | MI-3 (Menin-MLL Inhibitor) is a potent menin- | 1271738-59-0 |
| Inhibitor) | MLL interaction inhibitor with IC50 of 648 nM. | |
| MG149 | MG149 is a potent histone acetyltransferase inhibitor with | 1243583-85-8 |
| | IC50 of 74 μM and 47 μM for Tip60 and MOF,respectively. | |
| ML324 | ML324 is a selective inhibitor of jumonji histone | 1222800-79-4 |
| | demethylase (JMJD2) with IC50 of 920 nM. | |
| OF-1 | OF-1 is a potent inhibitor of BRPF1B and BRPF2 | 919973-83-4 |
| | bromodomain with K<sub>d</sub> of 100 nM and 500 nM, | |
| | respectively. | |
| 4SC-202 | 4SC-202 is a selective class I HDAC inhibitor with IC50 of | 910462-43-0 |
| | 1.20 μM, 1.12 μM, and 0.57 μM for HDAC1, HDAC2, and | |
| | HDAC3, respectively. Also displays inhibitory activity | |
| | against Lysine specific demethylase 1 (LSD1). Phase 1. | |
| NI-57 | NI-57 is a selective and potent inhibitor of BRPF | 1883548-89-7 |
| | (Bromodomain and PHD Finger) family of proteins | |
| | (BRPF1/2/3). NI-57 shows accelerated FRAP recovery at 1 | |
| | μM in the BRPF2 FRAP assay preventing binding of full- | |
| | length BRPF2 to chromatin. | |
| MS023 | MS023 is a potent and selective chemical probe for Type I | 1831110-54-3 |
| hydrochloride | protein arginine methyltransferases (PRMTs). MS023 is a | |
| | potent inihbitor of PRMTs 1, 3, 4, 6, and 8 (IC50 = 30, 119, | |
| | 83, 8, and 8 nM, respectively), which are responsible for | |
| | asymmetric dimethylation of arginine residues. MS023 is | |
| | active in cells. | |
| OICR-9429 | OICR-9429 is a cell penetrant, potent and selective | 1801787-56-3 |
| | antagonist of the interaction of WDR5 (WD repeat domain 5) | |
| | with peptide regions of MLL and Histone 3 that potently | |
| | binds to WDR5. OICR-9429 inhibits the interaction of | |
| | WDR5 with MLL1 and RbBP5 in cells. | |
| LLY-507 | LLY-507 is a potent and selective inhibitor of SMYD2 | 1793053-37-8 |
| | protein lysine methyltransferase (PKMT) with an in vitro | |
| | IC50 < 15 nM and >100-fold selectivity over other | |
| | methyltransferases and other non-epigenetic targets. LY- | |
| | 507 has been shown to inhibit p53K370 monomethylation | |
| | in cells with an IC50 ~ 600 nM. | |
| I-BRD9 | I-BRD9 is a selective cellular chemical probe for | 1714146-59-4 |
| | bromodomain-containing protein 9 (BRD9), thought to be | |
| | a member of the chromatin remodelling SWI/SNF BAF | |
| | complex, which plays a fundamental role in gene | |
| | expression regulation. I-BRD9 has a pIC50 value of 7.3 | |
| | with greater than 700-fold selectivity over the BET family | |
| | and 200-fold over the highly homologous bromodomain- | |
| | containing protein 7 (BRD7) and greater than 70-fold | |
| | selectivity against a panel of 34 bromodomains. | |
| SGC707 | SGC707 is a potent allosteric inhibitor of protein arginine | 1687736-54-4 |
| | methyltransferase 3 (PRMT3). SGC707 has an IC50 value | |
| | of 50 nM and >100-fold selectivity over other | |
| | methyltransferases and non-epigenetic targets. SGC707 | |
| | binds to PRMT3 with KD of 50 nM (ITC), and inhibits | |
| | histone methylation in cells with an IC50 value below 1 | |
| | μM. | |
| BAZ2-ICR | BAZ2-ICR is a chemical probe for BAZ2A/B bromodomains | 1665195-94-7 |
| | with >100-fold selectivity over other bromodomains, with | |
| | the exception of CECR2 (15-fold selectivity). BAZ2A is an | |
| | essential component of the nucleolar remodeling complex | |
| | (NoRC), which mediates recruitment of histone modifyine | |
| | enzymes and DNA methylase involved in the silencing of | |
| | ribosomal RNA transcription by RNA polymerase I. BAZ2B | |
| | is believed to be involved in regulating nucleosome | |
| | mobilization along linear DNA. BAZ2-ICR binds to BAZ2A | |
| | with a KD of 109 nM (ITC) and to BAZ2B with a KD of 170 | |
| | nM (ITC). BAZ2-ICR also shows accelerated Fluorescence | |
| A-366 | A-366 is an SGC chemical probe for G9a/GLP, developed in | 1527503-11-2 |
| | collaboration with Abbvie. A-366 is a potent, selective | |
| | inhibitor of the histone methyltransferase G9a. The IC50 | |
| | values for G9a inhbition in enzymatic and cell based assays | |
| | are 3.3 and approximately 3 μM, respectively. A-366 has little | |
| | or no detectable activity against a panel of 21 other | |
| | methyltransferases. | |
| MS049 | MS049 is a potent and selective inhibitor of protein arginine | 1502816-23-0 |
| hydrochloride | methyltransferases (PRMTs) PRMT 4 and PRMT6. MS049 | |
| | is active in cells. | |
| PFI-4 | PFI-4 is an SGC chemical probe for the bromodomains of | 900305-37-5 |
| | the BRPF (BRomodomain and PHD Finger containing) | |
| | scaffolding protein BRPF1B. The BRPF proteins | |
| | (BRPF1/2/3) assemble histone acetyltransferase (HAT) | |
| | complexes of the MYST transcriptional coactivator family | |
| | members MOZ and MORF. The BRPF1 protein is the | |
| | scaffold subunit of the MYST acetyltransferase complex, | |
| | which plays a crucial roles in DNA repair, recombination | |
| | and replication as well as transcription activation. Mutations | |
| | in MOZ, MORF, and BRPF1 have all been associated with | |
| | cancer. BRPF1 exists in 2 different isoforms: BRPF1A and | |
| | BRPF1B. PFI-4 specifically binds to BRPF1B with a Kd = | |
| | 13 nM as determined by ITC. It reduces recovery time in | |
| | triple BRD cell construct in FRAP and is potent in cells with | |
| | IC50 250nM, while showing no effect on BRPF1A. | |
| A-196 | A-196 is a potent and selective chemical inhibitor of | 1982372-88-2 |
| | SUV420H1 and SUV420H2 that inhibits the di- and | |
| | trimethylation of H4K20me in multiple cell lines. | |
| (+)-JQ1 | The human BET family, which includes BRD2, BRD3, | 1268524-69-1 |
| | BRD4 and BRDT, play a role in regulation of gene | |
| | transcription. (+)-JQ1 ((+)SGCBD01) is a selective BET | |
| | bromodomain (BRD) inhibitor that inhibits Brd4 | |
| | (Bromodomain-containing 4). Brd4 forms complexes with | |
| | chromatin via two tandem bromodomains (BD1 and BD2) | |
| | that bind to acetylated lysine residues in histones and Brd4 | |
| | association with acetylated chromatin is believed to regulate | |
| | the recruitment of elongation factor b and additional | |
| | transcription factors to specific promoter regions. The | |
| | nuclear protein in testis (NUT) gene can form fusions with | |
| | Brd4 that create a potent oncogene, leading to rare, but | |
| | highly lethal tumors referred to as NUT midline carcinomas | |
| | (NMC). JQ1 inhibits recruitment and binding of Brd4 to | |
| | TNFa and E-selectin promoter elements, and accelerates | |
| | recovery time in FRAP (fluorescence recovery after | |
| | photobleaching) assays using GFP-Brd4. Thus | |
| | JQ1/SGCBD01 is a useful tool to study the role of Brd4 in | |
| | transcriptional initiation. | |

[symbol]

indicates data missing or illegible when filed

The HDAC inhibitor romidepsin (also known as FK228) did not score positively in the screen. This was likely due to the relatively high concentrations tested, which were lethal to both RNF5-KD and control U937 cells. FK228 has been approved by the Food and Drug Administration (FDA) to treat peripheral T-cell lymphoma and has been investigated in preclinical studies as a potential treatment for AML. Therefore, FK228 was re-assessed at non-lethal concentrations (up to 6 nM for 24 h) using multiple AML cell lines. Notably, when combined with RNF5-KD, FK228 had an additive effect in decreasing cell viability (FIGS. 7D, 7E, and 14B-14D) and inducing apoptosis (FIG. 7F). It is confirmed that the additive effect of RNF5 loss plus treatment with HDACi in MOLM-13 cells made deficient in RNF5 using the CRISPR/Cas9 system (FIG. 14E). The additive effect on AML cell death of RNF5-KD plus FK228 treatment was lost following RNF5 re-expression (FIG. 7G), confirming a specific role for RNF5 in sensitizing AML cells to HDAC inhibition.

Of AML cell lines tested, the MV-4-11 line showed very low levels of RNF5 protein (FIG. 15B). MV-4-11 cells were also most sensitive to FK228 treatment (FIG. 14F), and RNF5 KD did not increase their sensitivity (FIG. 14G). These observations further support the importance of RNF5 abundance in the response of AML cells to HDAC inhibitors. Moreover, RBBP4 KD sensitized AML cells to FK228 treatment (FIGS. 7H and 14H), consistent with the findings that RNF5 positively regulates RBBP4. Notably, H3K9 acetylation at the promotors of RNF5- or RBBP4-regulated genes, such as ANXA1 and CDKN1A, increased following FK228 treatment and further increased upon RNF5 KD (FIG. 7I). The latter finding is consistent with increased ANXA1 and CDKN1A expression seem after treatment with FK228 alone or in combination with RNF5 KD (FIGS. 7J and 14I). Notably, RNF5 KD in the K562 (CIVIL) line did not sensitize cells to FK228, and RNF5 KD in Jurkat cells (T-ALL) only slightly enhanced cell sensitivity to FK228 (FIGS. 14J and 14K).

Next, to corroborate these findings in primary AML blasts, ex-vivo analysis of AML patients' samples (n=4) performed and their sensitivity to FK228 treatment was assessed. These samples were selected based on RNF5 and RBBP4 protein levels (2 high, 2 low). Surprisingly, and similar to phenotypes seen in the KD experiments, samples expressing low RNF5 and RBBP4 were more sensitive (AML-075B log IC₅₀=−10.7M, AML-037 log IC₅₀=−10.4M) to FK228 treatment, compared with high-expressing group (AML-013 log IC₅₀=−9.9M, AML-072B log IC₅₀=−9.6M) (FIGS. 7K-7M). Finally, the relevance of combined RNF5 loss and HDAC treatment in patients was analyzed using data from a bioinformatic pipeline that identifies clinically relevant synthetic lethal interactions. This analysis revealed a more favorable prognosis in patients with concomitant downregulation of both HDAC and RNF5 (FIG. 7N), substantiating the sensitization to HDAC in RNF5 low expressing specimens. Collectively, these findings suggest that RNF5 signaling is a critical determinant of AML cell sensitivity to HDAC inhibitors.

High mortality seen in patients with AML predominantly results from failure to achieve complete remission following chemotherapy, coupled with a high relapse rate. The current disclosure identifies an important role for the ubiquitin ligase RNF5 in AML and defines mechanistically how RNF5 contributes to this form of leukemia. The current disclosure establishes a function for RNF5 beyond its previously characterized activity in ERAD and proteostasis and demonstrates how it regulates gene expression programs governing AML development and response to HDAC inhibitors. The clinical relevance of RNF5 and RBBP4 to AML is supported by the findings based on patient samples and genetic mouse models. In mice, RNF5 or RBBP4 depletion inhibited AML progression and prolonged mouse survival (FIG. 4). In human, analysis of AML samples from two independent clinical cohorts revealed that high abundance of RNF5 protein, which is commonly seen in AML patient samples, correlates with poor prognosis. Those phenotypes are mediated via RNF5 interaction with the chromatin remodeling protein RBBP4, which results in its non-canonical (K29 topology) ubiquitination that promotes RBBP4 recruitment to specific gene promoters (among them, ANXA1, NCF1, and CDKN1A), and concomitant upregulation of genes implicated in AML maintenance. The finding that RNF5 modifies RBBP4 in a way that alters expression of AML-related genes is confirmed by ChIP analysis showing that RNF5 promotes recruitment of RBBP4 to gene promoters. Moreover, there is an inverse correlation between RBBP4 expression and expression of genes upregulated in RNF5 KD cells in TCGA database (FIG. 5). It is contemplated that genome-wide assessment of promoter-bound RBBP4 can be used to reveal additional genes whose transcription is regulated by RNF5-modified RBBP4. These additional genes can also be targets for pharmaceutical intervention, for example, by using inhibitors of these genes.

Independent support for a function for RNF5 in recruiting RBBP4 to transcriptional regulatory complexes comes from the finding that RNF5/RBBP4 abundance governs the sensitivity of AML cells to HDAC inhibitors. Correspondingly, transcriptional changes induced by RNF5 KD overlapped with those seen following treatment with HDAC1 inhibitors. Furthermore, AML primary blasts expressing low RNF5/RBBP4 levels were more sensitive to FK228 compared to high expressing blasts. Along these lines, synthetic lethal analysis also identified a favorable prognosis in a cohort of AML patients with low expression of both HDAC and RNF5 (FIG. 7). Thus, RNF5 or RBBP4 abundance may serve as useful markers for stratification of AML patients for treatment with HDAC inhibitors.

Notably, RNF5 is expressed at high levels in AML, CIVIL and T-ALL cell lines²⁰ but is critical for cell survival only in AML cells. In fact, the CCLE database reveals that CIVIL and T-ALL lines express on average higher levels of RNF5 than do AML lines. Nonetheless, K-562 (CIVIL) and Jurkat (T-ALL) lines subjected to RNF5 KD do not exhibit growth inhibition or undergo cell death, while similarly treated AML lines do. Likewise, inhibition of RBBP4 does not impact CIVIL or T-ALL cell but rather inhibits AML cell growth in a manner similar to that seen after RNF5 inhibition. Along these lines, RNF5 regulation and function are likely to be cell type and tissue dependent. RNF5 promotes melanoma growth via changes in immune and intestinal epithelial cells, while inhibits breast cancer growth through tumor-intrinsic expression of glutamine carrier proteins ^{7,8,38}.

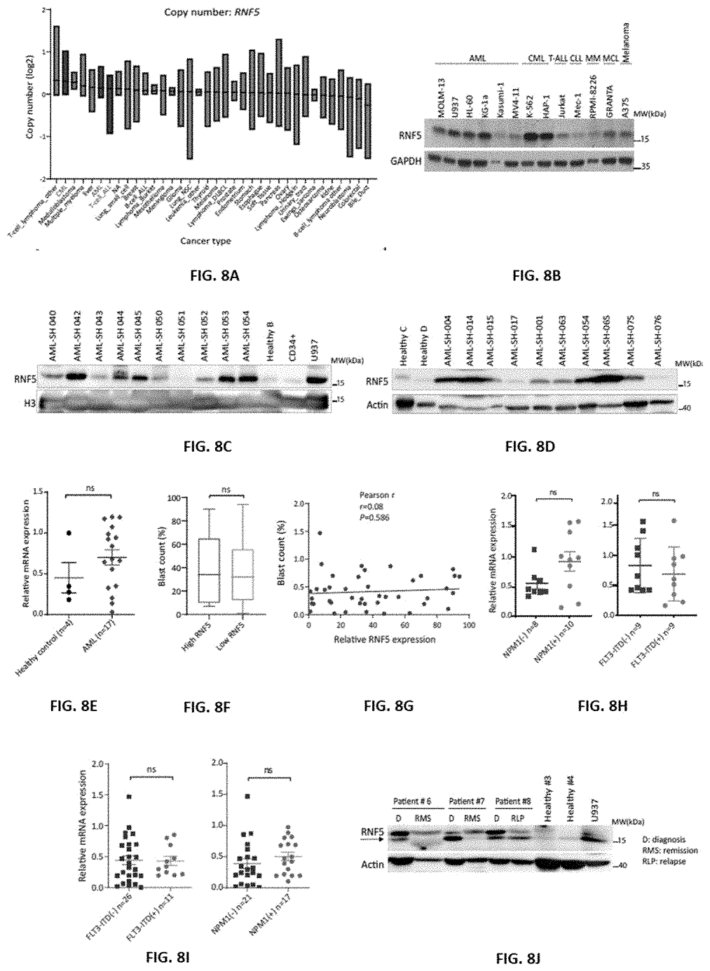

It is important to note that relatively high RNF5 expression in AML cell lines is likely due to high copy number, as shown by analysis of copy number alterations in various cancer cells²⁰. Analysis of the TCGA database reveals increases in RNF5 mRNA levels in 3% of patients. The patient cohorts revealed a significant increase in RNF5 abundance but not transcription levels (FIGS. 1B, 1C, and 8E). Thus, RNF5 overexpression (at the protein level) is attributable to relative increases in RNF5 protein stability, as supported by the assessments of two independent AML cohorts (FIG. 1). As a ubiquitin ligase, RNF5 activity is regulated primarily by self-degradation rather than transcription. However, the possibility that these increases are linked to a pre-existing mutation that could increase RNF5 abundance in AML patient PMBCs or to micro-vesicle-based cell-cell communication cannot be ruled out.

Given that RNF5 protein is ER-anchored, its interaction with a chromatin regulatory protein such as RBBP4 is unanticipated. This interaction may be trigger by one or more events, including, but not limited to: (i) the interaction may occur as the RBBP4 gene is translated, prior to nuclear translocation, a mechanism reported for other ubiquitin ligases³⁹, (ii) a post translational modification may promote nuclear localization of RNF5. For example, the possibility that RNF5 undergoes sumoylation should be considered given the high probability predicted using GPS-SUMO tool ⁴⁰; Finally, or (iii) RBBP4/RNF5 interaction may occur at specific phases of the cell cycle, for example, at entry to mitosis, when the nuclear envelop breaks down and nuclear contents are released to the cytoplasm.

In summary, genetic mouse models and clinical data in the current application establish a central role for the RNF5-RBBP4 axis in AML maintenance and responsiveness to HDAC inhibitors. The identification of a crosstalk between ubiquitination and epigenetic regulation offers a new paradigm for ERAD-independent RNF5 function in controlling RBBP4 activity and subsequent transcriptional networks implicated in AML. The current application also demonstrates the ability of HDAC inhibitors to treat AML, particularly AML expressing low levels of RNF5, and provides a method to stratify AML patients for treatment with HDAC inhibitors.

Animal studies. All animal experiments were approved by the Sanford Burnham Prebys Medical Discovery Institute's Institutional Animal Care and Use Committee (approval AUF 16-028). Animal care followed institutional guidelines. Rnf5^{-/-} mice were generated on a C57BL/6 background as described 23. C57BL/6 WT mice were obtained by crossing Rnf5^{+/-} mice. Female mice were maintained under controlled temperature (22.5° C.) and illumination (12 h dark/light cycle) conditions and were used in experiments at 6-10 weeks of age.

The xenograft model was established using U937 cells expressing the p-GreenFirel Lenti-Reporter Vector (pGFL). NOD/SCID (NOD.CB17-Prkdcscid/J) mice were obtained from the SBP Animal Facility. Mice were irradiated (2.5 Gy), and U937-pGFL cells (2×10⁴ per mouse) were injected intravenously. Leukemia burden was serially assessed using noninvasive bioluminescence imaging by injecting mice intraperitoneally (i.p.) with 150 mg/kg D-Luciferin (PerkinElmer, 122799) in phosphate-buffered saline (PBS, pH 7.4), anesthetizing them with 2-3% isoflurane, and imaging them on an IVIS Spectrum (PerkinElmer). For in vivo RNF5 KD experiments, at disease onset (day 15, as measured by bioluminescent imaging), mice were fed rodent chow containing 200 mg/kg doxycycline (Dox diet, Bio-Serv) to induce RNF5-KD. Mice were sacrificed upon signs of morbidity resulting from leukemic engraftment (>10% weight loss, lethargy, and ruffled fur).

Cell culture. Human HEK293T and A375 cells were obtained from the American Type Culture Collection (ATCC). U937 and K562 cells were kindly provided by Prof. Yuval Shaked; Kasumi-1 cells were from Prof. Tsila Zuckerman; and MV4-11, GRANTA, THP-1, and MEC-1 cells were from Dr. Netanel Horowitz. MOLM-13 cells were kindly provided by Dr. Ani Deshpande (SBP Discovery Institute, USA), KG-1a, HL-60, Jurkat, RPMI 8226, and HAP-1 cells were a kind gift from Prof. Ciechanover (Technion, Israel). MOLM-13, U937, THP-1, Kasumi, Jurkat, and RPMI-8226 cells were cultured in RPMI medium; HL-60, MV-4-11, K-562, MEC-1, HAP-1, and KG-1α cells were cultured in IMDM; and GRANTA, A375 and HEK293T cells were cultured in DMEM. All media were supplemented with 10% fetal bovine serum (FBS), 1% L-glutamine, penicillin (83 U/mL), and streptomycin (83 μg/mL) (Gibco). Cells were regularly checked for mycoplasma contamination using a luminescence-based kit (Lonza).

Primary AML cells. AML patient samples were obtained from Scripps MD Anderson, La Jolla, Calif. (IRB-approved protocol 13-6180) and written informed consent was obtained from each participant. Samples were also obtained from the Rambam Health Campus Center, Haifa, Israel (IRB-approved protocol 0372-17). Fresh blood samples were obtained by peripheral blood draw, PICC line, or central catheter. Filgrastim-mobilized peripheral blood cells were collected from healthy donors and cryopreserved in DMSO. PBMCs were isolated by centrifugation through Ficoll-Paque™ PLUS (17-1440-02, GE Healthcare). Residual red blood cells were removed using RBC Lysis Buffer for humans (Alfa Aesar, cat. #J62990) according to the manufacturer's instructions. The final PBMC pellets were resuspended in Bambanker serum-free freezing medium (Wako Pure Chemical Industries, Ltd.) and stored under liquid N2. Patients' characteristics are provided in Table 1.

MLL-AF9 patient-derived xenograft (PDX) samples (from the Jeremias Lab, Munich, Germany) were cultured in IMDM medium with 20% BIT (Stem cell Technologies), human cytokines and StemRegenin 1 (SR1) and UM171, as described 41. Cells were transduced with empty vector or different shRNF5 constructs as described below (see Transfections and transduction section) and plated in 100 uL per well of complete medium in 96-well plates. Growth was monitored every 24 h using CellTiter Glo reagent.

For the drug dose responses, FK228 was diluted in DMSO at 10 mM and serially diluted (1/3, ×13 concentrations) in a Labcyte Echo Low Dead Volume (LDV) plate. 25 nLs of drugs at 1000× concentration were spotted in quadruplicate in 384-well plates (Greiner #781098) using a Labcyte Echo 550 acoustic dispenser, and patient AML cells (described above) were seeded (2.5 k cells/well in 25 uLs) onto 3 plates with a Multidrop Combi Reagent Dispenser (Thermo). After 2 days, cell viability was assessed by adding 10 uLs/well of CellTiterGlo reagent (Promega #G7572) using a Multidrop Combi, and luminescence was read on an Envision plate reader (Perkin Elmer). Raw data was processed in Microsoft Excel, with cell viability values normalized to percentages relative to vehicle (0.1% DMSO) controls. Data were graphed and subjected to statistical analyses using GraphPad Prism software (v.9.1.1).

Antibodies and reagents. The RNF5 antibody was produced as described previously (1:1000) 7′23. Other antibodies used were: rabbit anti-cleaved caspase 3 (#9661, Cell Signaling Technology, 1:1000), rabbit anti-PARP (#9532, Cell Signaling Technology, 1:1000), mouse anti-RBBP4 (NBP1-41201, Novus Biologicals, 1:5000), mouse anti-glyceraldehyde 3-phosphate dehydrogenase (GAPDH; ab8245, Abcam, 1:10000), mouse anti-Tubulin (T9026, Sigma, 1:5000), mouse anti-Flag (F1804, Sigma, 1:2000), mouse anti-Myc-Tag (#2276, Cell Signaling Technology, 1:1000), mouse anti-HA (901501, Biolegend, 1:2000), rabbit anti-HDAC1 (#2062, Cell Signaling Technology, 1:1000), rabbit anti-HDAC2 (57156, Cell Signaling Technology, 1:1000), rabbit anti-Ezh2 (5246, Cell Signaling Technology, 1:1000), mouse anti-HSP90 (sc-13119, Santa Cruz Biotechnology, 1:1000), rabbit anti-p27, (#3688, Cell Signaling Technology, 1:1000), rabbit anti-p21 (#2947, Cell Signaling Technology, 1:1000), mouse anti-Ubiquitin (#3939, Cell Signaling Technology, 1:1000), rabbit anti-K63-linkage Specific Polyubiquitin (#5621, Cell Signaling Technology, 1:1000), rabbit anti-Actin (#4970, Cell Signaling Technology, 1:1000), rabbit anti-Histone H3 (#9717, Cell Signaling Technology, 1:1000), mouse anti-Caspase 3 (sc-56053, Santa Cruz Biotechnology, 1:1000), and mouse anti-Calregulin (sc-166837, Santa Cruz Biotechnology, 1:1000). HRP-conjugated secondary antibodies were from Jackson ImmunoResearch (goat-anti-mouse-HRP (AB_2338504) and goat-anti-rabbit-HRP (AB_2337938) and diluted 1:10000.

Romidepsin and N-acetyldinaline were purchased from Cayman Chemicals. Thapsigargin and tunicamycin were purchased from Sigma-Aldrich. MG132 was obtained from Selleckchem. Puromycin was purchased from Merck. Annexin V-APC and propidium iodide were from BioLegend.

Plasmids and constructs. Plasmids expressing Flag-RNF5-WT, Flag-RNF5-RM, and Flag-RNF5-ACT were described previously ^{5,7}. To generate doxycycline-inducible RNF5-WT, RNF5-RM, and RNF5-ACT overexpression vectors, coding sequences were amplified by PCR from pCDNA3.1-RNF5-WT, pCDNA3.1-RNF5-RM, and pCDNA3.1-RNF5-ACT, respectively, and the product was inserted into EcoRI-linearized pLVX TetOne-puro plasmid (Clontech) using the NEBuilder HiFi Assembly kit (New England BioLabs). Expression vectors encoding Myc-RBBP4 (#20715), HA-Ubiquitin (#18712), and HA-ubiquitin mutants (including K6 (#22900), K11 (#22901), K27 (#22902), K29 (#22903), and K33 (#17607)) were obtained from Addgene.

Gene silencing. Lentiviral pLKO.1 vectors expressing RNF5 or RBBP4-specific shRNAs were obtained from the La Jolla Institute for Immunology RNAi Center (La Jolla, Calif., USA). Sequences were: shRNF5 #1 (TRCN0000004785) GAGTGTCCAGTATGTAAAGCT (SEQ ID NO: 35), shRNF5 #2 (TRCN0000004788) CGGCAAGAGTGTCCAGTATGT (SEQ ID NO: 36), shRNF5 #3 GAGGATGGATTGAGAGAAT (SEQ ID NO: 37), and inducible shRNF5, which has the same sequence as shRNF5 #1. Sequences for RBBP4-specific shRNAs were: shRBBP4 #1 (TRCN0000286103) GCCTTTCTTTCAATCCTTATA (SEQ ID NO: 38), shRBBP4 #2 (TRCN0000293556) TGGTCATACTGCCAAGATATC (SEQ ID NO: 39), shRBBP4 #3 (TRCN0000293554) ATGCGTCACACTACGACAGTG (SEQ ID NO: 40).

Transfections and transduction. Transient transfections were carried out using CalFectin (SignaGen) according to the manufacturer's recommendations. Lentiviral particles were prepared using standard protocols. In brief, HEK293T cells were transfected with relevant vectors and the second-generation packaging plasmids AR8.2 and Vsv-G (Addgene). Virus-containing supernatants were collected 48 h later and then added in the presence of Polybrene to AML cells pre-seeded at ˜5×10⁵/well in 24-well plates (Sigma-Aldrich). After 8 h, cells were transferred to 10-cm culture dishes for an additional 24 h prior to experiments.

Western blotting. Cells were washed twice with cold PBS and lysed by addition of Tris-buffered saline (TBS)-lysis buffer (TBS [50 mM Tris-HCl pH 7.5, 150 mM NaCl], 0.5% Nonidet P-40, 1× protease inhibitor cocktail [Merck], and 1× phosphatase inhibitor cocktail ⁴² followed by incubation on ice for 20 min. Blood cells from healthy control subjects and AML patients were lysed using hot lysis buffer [100 mM Tris-HCl pH 7.5, 5% sodium dodecyl sulfate (SDS)] followed by incubation 5 min at 95° C. and sonication. Some samples were subjected to fractionation using a subcellular protein fractionation kit (Thermo Scientific Pierce), as indicated. Samples were resolved on SDS-PAGE and transferred to nitrocellulose membranes. Membranes were incubated for 1 h at room temperature with blocking solution (0.1% Tween 20/5% non-fat milk in TBS) and then overnight at 4° C. with primary antibodies. Membranes were washed with TBS and incubated 1 h at room temperature with appropriate secondary antibodies (Jackson ImmunoResearch). Finally, proteins were visualized using a chemiluminescence method (Image-Quant LAS400, GE Healthcare, or ChemiDoc MP imaging system, Bio-Rad). The uncropped scans for all western blot are provided in the Source Data file.

Immunoprecipitation. Cells were lysed in TBS-lysis buffer as described above, centrifuged for 10 min at 17,000 g, and incubated overnight at 4° C. with appropriate antibodies. Protein A/G agarose beads (Santa Cruz Biotechnology) were then added for 2 h at 4° C. Beads were pelleted by centrifugation, washed five times with TBS-lysis buffer, and boiled in Laemmli buffer to elute proteins. Finally, proteins were resolved on SDS-PAGE and subjected to Western blotting as described above.

LC-MS/MS. MOLM-13 cells were infected with doxycycline-inducible Flag-tagged RNF5-encoding or empty plasmids and expression was induced by addition of doxycycline (1 μg/mL) for 48 h. The proteasome inhibitor MG132 (10 μM) was added for 4 h prior to harvest. Cells were lysed in TBS-lysis buffer, and total cell lysates were incubated with anti-Flag-M2-agarose beads (Sigma-Aldrich) overnight at 4° C. Beads were washed with TBS-lysis buffer, and proteins were eluted from beads by addition of 3×Flag peptides (150 μg/mL, Sigma) for 1 h at 4° C. and then subjected to tryptic digestion followed by LC-MS/MS, as described⁴³.

Raw data were analyzed using MaxQuant (v1.5.5.0)⁴⁴ with default settings. Protein intensities were normalized using the median centering method. Fold-changes were calculated by dividing protein intensity of Flag immunoprecipitates from RNF5-overexpressing cells by the protein intensity of Flag immunoprecipitates from control cells. Thresholds were set at 2 for fold-change and 0.05 for p value obtained using a two-sided Welch's t-test. Proteins identified in all RNF5 immunoprecipitation replicates but in one or no control IP replicates were considered potential RNF5 interactors if their corresponding fold-change was at least 2. Data from the Crapome (version 2.0)⁴² repository were downloaded to filter potential contaminants. Cytoscape (version 3.8.1)₄₅ was used to generate the RNF5 interaction network and pathway enrichment analysis. Raw MS data were deposited in the MassIVE repository under the accession code MSV000083160.

Immunofluorescence microscopy. Cells were placed on coverslips on glass slides using a StatSpin cytofuge and fixed with 4% paraformaldehyde for 20 min at room temperature. Slides were then rinsed three times in PBS, and cells were permeabilized in 0.2% Triton X-100 in PBS for 5 min and blocked with 0.2% TX-100/10% FBS in PBS for 30 min. Primary antibodies were diluted in staining buffer (0.2% Triton X-100/2% FBS in PBS) and added to cells, and the slides were then incubated overnight at 4° C. in a humidified chamber. Slides were then washed three times in staining buffer, and secondary antibodies (Life Technologies) were diluted in staining buffer and added to slides for 1 h at room temperature in a humidified chamber shielded from light. Finally, slides were washed three times in staining buffer and mounted with Fluoroshield Mounting Medium containing 4′, 6-diamidino-2-phenylindole (DAPI; Sigma-Aldrich). Cells were analyzed using a fluorescence microscope (DMi8; Leica) with a 60× oil immersion objective. Images were processed using the 3D deconvolution tool from LASX software (Leica), and the same parameters were used to analyze all images.

Cell viability assay. Cell viability and growth were assayed using the CellTiter Glo kit (Promega) according to the manufacturer's recommendations. Cell lines were plated in white 96-well clear-bottomed plates (Corning) at a density of 7×10³ cells/well, and growth was monitored every 24 h using CellTiter Glo reagent. Viability was quantified by measuring luminescence intensity with an Infinite 2000 Pro reader (Tecan).

Cell cycle analysis. Distribution of cells in each phase of the cell cycle was analyzed by propidium iodide staining (Merck). Briefly, 1×10⁶ cells were washed twice with cold PBS and fixed in 70% ethanol in PBS at 4° C. overnight. Cells were washed, pelleted by centrifugation, and treated with RNase A (100 μg/mL) and propidium iodide (40 μg/mL) at room temperature for 30 min. Cell cycle distribution was assessed by flow cytometry (BD LSRFortessa™, BD Biosciences), and data was analyzed using FlowJo software.

Annexin V and propidium iodide staining. Cells were collected in FACS tubes, washed twice with ice-cold PBS, and resuspended in 100 μL PBS. Annexin V-APC (1.4 μg/mL) was added for 15 min at room temperature in the dark. Then, cells were washed in PBS and resuspended in 200 μL PBS, and then propidium iodide (50 μg/mL) was added. Finally, samples were analyzed by flow cytometry (BD LSRFortessa™, BD Biosciences). Gating strategy is provided in FIG. 15C.

Colony-forming assays. For the soft agar assay, a base layer was formed by mixing 1.5% soft agar (low-melting point agarose, Bio-Rad) and culture medium at a 1:1 ratio and placing the mixture in 6-well plates. Cells were resuspended in medium containing 0.3% soft agar and added to the base layer at 1×10⁴ (MOLM-13) or 5×10³ (U937) cells/well. Agar was solidified by incubation at 4° C. for 10 mins before incubation at 37° C. Plates were incubated at 37° C. in a humidified atmosphere for 12-18 days. Cells were then fixed overnight with 4% paraformaldehyde, washed with PBS, and stained with 0.05% crystal violet (Merck) for 20 min at room temperature and washed again with PBS. Plates were photographed and colonies were counted on the captured images.

For the methylcellulose assay, WT or Rnf5^{-/-}Lin⁺Sca1⁺c-Kit⁺ cells transformed with GFP-MLL-AF9 were resuspended in methylcellulose M3234 (Stem Cell Technologies) supplemented with 6 ng/mL IL-3, 10 ng/mL IL-6, and 20 ng/mL stem cell factor (PeproTech). Cells were then added to 35-mm dishes at 10³ cells/well and incubated for 6-7 days. Colonies were classified as compact and hypercellular (blast-like) or small and diffuse (differentiated). Virtually all colonies fell into one of these two categories.

RT-qPCR analysis. RNA was extracted using a GenElute Mammalian Total RNA Purification Kit (Sigma) according to standard protocols. RNA concentration was measured using a NanoDrop spectrophotometer (ThermoFisher). cDNA was synthesized from aliquots of 1 μg total RNA using a qScript cDNA Synthesis Kit (Quanta). Quantitative PCR was performed with SYBR Green I dye master mix (Roche) and a CFX connect Real-Time PCR System (Bio-Rad). Primer sequences are listed in Table 3. Primer efficiency was measured in preliminary experiments, and amplification specificity was confirmed by dissociation curve analysis.

TABLE 3
List of primers used for RT-qPCR analysis

| Gene | Forward | Reverse |
|---|---|---|
| RNF5 | AAAGCTGGGATCAGCAGAGA (SEQ | ATCACCAAATGGCTGGAATC (SEQ ID |
| | ID NO: 1) | NO: 2) |
| ANXA1 | ATACAGATGCCAGGGCTTTGTATGA | TGGGATGTCTAGTTTCCACCACACA |
| | (SEQ ID NO: 3) | (SEQ ID NO: 4) |
| H3A | AAGCAGACTGCCCGCAAAT (SEQ ID | GGCCTGTAACGATGAGGTTTC (SEQ ID |
| | NO: 5) | NO: 6) |
| SXBP1 | GCTGGCAGGCTCTGGGGAAG (SEQ | TGCTGAGTCCGCAGCAGGTG (SEQ ID |
| | ID NO: 7) | NO: 8) |
| CHOP | GGAAACAGAGTGGTCATTCCC (SEQ | CTGCTTGAGCCGTTCATTCTC (SEQ ID |
| | ID NO: 9) | NO: 10) |
| ATF3 | CCTCTGCGCTGGAATCAGTC (SEQ ID | TTCTTTCTCGTCGCCTCTTTTT (SEQ ID |
| | NO: 11) | NO: 12) |
| LIMK1 | CAAGGGACTGGTTATGGTGGC (SEQ | CCCCGTCACCGATAAAGGTC (SEQ ID |
| | ID NO: 13) | NO: 14) |
| CDKN2D | AGTCCAGTCCATGACGCAG (SEQ ID | ATCAGGCACGTTGACATCAGC (SEQ ID |
| | NO: 15) | NO: 16) |
| CDKN1A | TGTCCGTCAGAACCCATGC (SEQ ID | AAAGTCGAAGTTCCATCGCTC (SEQ ID |
| | NO: 17) | NO: 18) |
| BCL2A1 | CTGCACCTGACGCCCTTCACC (SEQ | CACATGACCCCACCGAACTCAAAGA |
| | ID NO: 19) | (SEQ ID NO: 20) |
| NCF1 | GGGGCGATCAATCCAGAGAAC (SEQ | GTACTCGGTAAGTGTGCCCTG (SEQ ID |
| | ID NO: 21) | NO: 22) |
| YWHAZ | ACTTTTGGTACATTGTGGCTTCAA | CCGCCAGGACAAACCAGTAT (SEQ ID |
| | (SEQ ID NO: 23) | NO: 24) |

Gene targeting using CRISPR/Cas9. RNF5 sgRNAs were cloned into the pKLV2-U6gRNA-(BbsI)-PGKpuro2ABFP-W lentiviral expression vector and transduced into Cas9-expressing cell lines. All gRNAs were cloned into the BbsI site of the gRNA expression vector as previously described 46. Briefly, HEK293T cells were co-transfected with pKLV2-U6gRNA-(BbsI)-PGKpuro2ABFP-W and ectopic packaging plasmids using CalFectin transfection reagent (SignaGen). Virus-containing supernatants were collected 48 h later. MOLM-13 cells were infected by addition of supernatants for 48 h. Cells were then selected with puromycin (0.5 μg/mL) for 48 h and viability was measured. The RNF5-targeting sgRNA sequences were: sgRNF5 #3 F-GCACCTGTACCCCGGCGGAA (SEQ ID NO: 25), and R-TTCCGCCGGGGTACAGGTGC (SEQ ID NO: 26), and sgRNF5 #4 F-GTTCCGCCGGGGTACAGGTG (SEQ ID NO: 27), and R-CACCTGTACCCCGGCGGAAC (SEQ ID NO: 28).

RNA-seq analysis. PolyA RNA was isolated using the NEBNext Poly(A) mRNA Magnetic Isolation Module, and bar-coded libraries were constructed using the NEBNext Ultra™ Directional RNA Library Prep Kit for Illumina (NEB, Ipswich, Mass.). Libraries were pooled and single end-sequenced (lx 75) on the Illumina NextSeq 500 using the High output V2 kit (Illumina, San Diego, Calif.). Quality control was performed using Fastqc (v0.11.5, Andrews S. 2010), reads were trimmed for adapters, low quality 5′ bases, and minimum length of 20 using CUTADAPT (v1.1). The number of reads per sample and the number of aligned reads suggested that read quality and number were good and that the data were valid for analysis. High-quality data were then mapped to human reference genome (hg19) using STAR mapping algorithm (version 2.5.2a) 47. Feature Counts implemented in Subread (v1.50)⁴⁸ was used to count the sequencing reads from mapped BAM files. Analyses of differentially expressed genes was subsequently performed using a negative binomial test method (edgeR, v3.34.0)⁴⁹ implemented using SARTools R Package (v1.2.0) 5°. A list of the differentially expressed genes was exported into excel file, and pathway analysis was performed by uploading the lists of differentially expressed genes to IPA (http://www.ingenuity.com) using the following criteria: |log 2(fold change)|>0.4 and P value <0.05. P values were determined using “Negative Binomial Generalized Linear Model (two sided)” to generate DEGs list. Multiple comparisons were also applied based on the “Benjamini & Hochberg” method. LINCS database 51 and other public data sets were processed by IPA. Molecular signatures for canonical pathways, upstream regulators, and causal networks were generated for each data set by IPA. Enrichment results in this study were compared to the LINCS molecular signatures by Analysis Match using z-scores developed by IPA. The z-scores represent how well activated or inhibited entities match data sets (% similarity). The top matched experiments in LINCS were selected by ranking the overall z-scores.

Chromatin immunoprecipitation (ChIP). ChIP analysis was performed using a ChIP Assay Kit (Millipore) following the manufacturer's instructions. Cells were fixed in 1% formaldehyde in PBS for 10 minutes at 37° C. Briefly, 1×10⁶ cells were used for each reaction. Cells were fixed in 1% formaldehyde at 37° C. for 10 minutes, and nuclei were isolated with nuclear lysis buffer (Millipore) supplemented with a protease inhibitor cocktail (Millipore). Chromatin DNA was sonicated and sheared to a length between 200 bp and 1000 bp. Sheared chromatin was immunoprecipitated at 4° C. overnight with anti-H3K9ac (9649, Cell Signaling Technology), anti-H3K27ac (ab3594, Abcam), anti-H3K27me3 (9733, Cell Signaling Technology), anti-RBBP4 (NBP1-41201, Novus). IgG was used as a negative control and anti-RNA polII (Millipore) served as a positive control antibody. Protein A/G bead-antibody/chromatin complexes were washed with low salt buffer, high salt buffer, LiCl buffer, and then TE buffer to eliminate nonspecific binding. Protein/DNA complexes were reverse cross-linked, and DNA was purified using NucleoSpin®. Levels of ChIP-purified DNA were determined by qPCR (see Table 4 for primer sequences). Relative enrichments of the indicated DNA regions were calculated using the Percent Input Method according to the manufacturer's instructions and are presented as % input.

TABLE 4
List of primers used for ChIP analysis

| Gene | Forward | Reverse |
|---|---|---|
| ANXA1 | TCACTTTGTTTTTGGACATAGCTGA | CCACACCTAGCAACCAGAAGTTAG |
| | (SEQ ID NO: 29) | (SEQ ID NO: 30) |
| NCF1 | TCATGCCTGTAATCCCAACA (SEQ ID | CTCTGCCTTCCAGGTTCAAG (SEQ ID |
| | NO: 31) | NO: 32) |
| CDKN1A | GGTGTCTAGGTGCTCCAGGT (SEQ ID | GCACTCTCCAGGAGGACACA (SEQ ID |
| | NO: 33) | NO: 34) |

Small molecule epigenetic regulator screen. Aliquots of compounds (10 mM in DMSO) from a library of 261 epigenetic regulators were dispensed at final concentrations of 0.5 μM or 5 μM into the wells of a Greiner (Monroe, N.C., Cat #781080) 384-well TC-treated black plate using a Labcyte Echo 555 acoustic pipette (Labcyte, San Jose, Calif.). U937 cells expressing an inducible shRNF5 vector were induced with doxycycline for 72 h and dispensed into the prepared plates at a density of 5×10²/well in 50μ.L RPMI-based culture medium (described above) using a Multidrop Combi (Thermo Fisher Scientific, Pittsburgh, Pa.). Plates were briefly centrifuged at −100 g and incubated at 37° C. with 5% CO₂ for 6 more days using MicroClime Environmental lids (Labcyte, San Jose, Calif.). Plates were placed at room temperature for 30 min to equilibrate, 20 μL/well CellTiter-Glo Luminescent Cell Viability Assay reagent (Promega, Madison, Wis.) was added using a Multidrop Combi, and plates were analyzed with an EnVision multimode plate reader (PerkinElmer, Waltham, Mass.).

For the analysis, the intensity of induced shRNF5-expressing cells was divided by the intensity of uninduced cells. Ratios were log₂ transformed and thresholds were calculated based on distribution of the log₂ ratios. The upper threshold was calculated as the Q3+1.5×Q, where Q3 is the third quartile and IQ is the interquartile. The lower threshold was calculated as the Q1-1.5×IQ, where Q1 is the first quartile. Ratios outside these thresholds were considered outliers from the global ratio distribution and thus were potential candidates for having a differential effect on RNF5-KD or control cells.

MLL-AF9-mediated transformation of bone marrow cells and generation of MLL-AF9-leukemic mice. HEK293T cells were co-transfected with Murine Stem Cell Virus (MSCV)-based MLL-AF9 IRES-GFP ²² and ectopic packaging plasmids. Viral supernatants were collected 48 h later and added to Lin⁻Sca-1⁺c-Kit⁺ cells isolated from bone marrow of WT or Rnf5^{-/-} C57BL/6 mice. Transduced cells were maintained in DMEM supplemented with 15% FBS, 6 ng/mL IL-3, 10 ng/mL IL-6, and 20 ng/mL stem cell factor, and transformed cells were selected by sorting for GFP⁺ cells. To generate “primary AML mice,” GFP-MLL-AF9-transduced cells were resuspended in PBS at 1×10⁶ cells/200 μL and injected intravenously into sub-lethally irradiated (650 Rad) 6- to 8-week-old C57BL/6 female mice.

Statistical analysis. Differences between two groups were assessed using two-tailed unpaired or paired t-tests or Wilcoxon rank-sum test, and differences between group means were evaluated using t-tests or ANOVA. Two-way ANOVA with Tukey's multiple comparison test was used to evaluate experiments involving multiple groups. Survival was analyzed by the Kaplan-Meier method and evaluated with a log-rank test. All data were analyzed using GraphPad Prism version 8 or 9 (GraphPad, La Jolla, Calif., USA) and expressed as means±SD or SEM. P<0.05 was considered significant. NS stands for not statistically significant.

### REFERENCES

1 Cancer Genome Atlas Research, N. et al. Genomic and epigenomic landscapes of adult de novo acute myeloid leukemia. N Engl J Med 368, 2059-2074, doi:10.1056/NEJMoa1301689 (2013).
2 Mrozek, K., Heerema, N. A. & Bloomfield, C. D. Cytogenetics in acute leukemia. Blood Rev 18, 115-136, doi:10.1016/50268-960X(03)00040-7 (2004).
3 Shen, Y. et al. Gene mutation patterns and their prognostic impact in a cohort of 1185 patients with acute myeloid leukemia. Blood 118, 5593-5603, doi:10.1182/blood-2011-03-343988 (2011).
4 Dohner, H. et al. Diagnosis and management of acute myeloid leukemia in adults: recommendations from an international expert panel, on behalf of the European LeukemiaNet. Blood 115, 453-474, doi:10.1182/blood-2009-07-235358 (2010).
5 Tcherpakov, M. et al. Regulation of endoplasmic reticulum-associated degradation by RNF5-dependent ubiquitination of JNK-associated membrane protein (JAMP). J Biol Chem 284, 12099-12109, doi:10.1074/jbc.M808222200 (2009).
6 Younger, J. M. et al. Sequential quality-control checkpoints triage misfolded cystic fibrosis transmembrane conductance regulator. Cell 126, 571-582, doi:10.1016/j.cell.2006.06.041 (2006).
7 Bromberg, K. D. et al. Increased expression of the E3 ubiquitin ligase RNF5 is associated with decreased survival in breast cancer. Cancer Res 67, 8172-8179, doi:10.1158/0008-5472.CAN-07-0045 (2007).
8 Jeon, Y. J. et al. Regulation of glutamine carrier proteins by RNF5 determines breast cancer response to ER stress-inducing chemotherapies. Cancer Cell 27, 354-369, doi:10.1016/j.ccell.2015.02.006 (2015).
9 Kuang, E. et al. Regulation of ATG4B stability by RNF5 limits basal levels of autophagy and influences susceptibility to bacterial infection. PLoS Genet 8, e1003007, doi:10.1371/journal.pgen.1003007 (2012).
10 Fujita, Y. et al. Regulation of S100A8 Stability by RNF5 in Intestinal Epithelial Cells Determines Intestinal Inflammation and Severity of Colitis. Cell Rep 24, 3296-3311 e3296, doi:10.1016/j.celrep.2018.08.057 (2018).
11 Garcia-Manero, G. et al. Phase 1 study of the oral isotype specific histone deacetylase inhibitor MGCD0103 in leukemia. Blood 112, 981-989, doi:10.1182/blood-2007-10-115873 (2008).
12 Masetti, R., Serravalle, S., Biagi, C. & Pession, A. The role of HDACs inhibitors in childhood and adolescence acute leukemias. J Biomed Biotechnol 2011, 148046, doi:10.1155/2011/148046 (2011).
13 Redner, R. L., Wang, J. & Liu, J. M. Chromatin remodeling and leukemia: new therapeutic paradigms. Blood 94, 417-428 (1999).
14 Bhalla, K. N. Epigenetic and chromatin modifiers as targeted therapy of hematologic malignancies. J Clin Oncol 23, 3971-3993, doi:10.1200/JCO.2005.16.600 (2005).
15 Millard, C. J. et al. The structure of the core NuRD repression complex provides insights into its interaction with chromatin. Elife 5, e13941, doi:10.7554/eLife.13941 (2016).
16 Zhang, Y., Iratni, R., Erdjument-Bromage, H., Tempst, P. & Reinberg, D. Histone deacetylases and SAP18, a novel polypeptide, are components of a human Sin3 complex. Cell 89, 357-364, doi:10.1016/s0092-8674(00)80216-0 (1997).
17 Guo, Q. et al. Expression of HDAC1 and RBBP4 correlate with clinicopathologic characteristics and prognosis in breast cancer. Int J Clin Exp Pathol 13, 563-572 (2020).
18 Li, Y. D., Lv, Z., Xie, H. Y. & Zheng, S. S. Retinoblastoma binding protein 4 up-regulation is correlated with hepatic metastasis and poor prognosis in colon cancer patients. Hepatobiliary Pancreat Dis Int 18, 446-451, doi:10.1016/j.hbpd.2019.08.006 (2019).
19 Kitange, G. J. et al. Retinoblastoma Binding Protein 4 Modulates Temozolomide Sensitivity in Glioblastoma by Regulating DNA Repair Proteins. Cell Rep 14, 2587-2598, doi:10.1016/j.celrep.2016.02.045 (2016).
20 Heckl B C, Carlet M, Vick B, Roolf C, Alsadeq A, Grunert M, Liu W H, Liebl A, Hiddemann W, Marschalek R, Schewe D M, Spiekermann K, Junghanss C, Jeremias I. Frequent and reliable engraftment of certain adult primary acute lymphoblastic leukemias in mice. Leuk Lymphoma. March (2019). 60(3):848-851. doi: 10.1080/10428194.2018.1509314. Epub 2018 Sep. 20.
21 Breckenridge, D. G., Germain, M., Mathai, J. P., Nguyen, M. & Shore, G. C. Regulation of apoptosis by endoplasmic reticulum pathways. Oncogene 22, 8608-8618, doi:10.1038/sj.onc.1207108 (2003).
22 Krivtsov, A. V. et al. Transformation from committed progenitor to leukaemia stem cell initiated by MLL-AF9. Nature 442, 818-822, doi:10.1038/nature04980 (2006).
23 Delaunay, A. et al. The ER-bound RING finger protein 5 (RNF5/RMA1) causes degenerative myopathy in transgenic mice and is deregulated in inclusion body myositis. PLoS One 3, e1609, doi:10.1371/journal.pone.0001609 (2008).
24 Pan, L. et al. DHX15 is associated with poor prognosis in acute myeloid leukemia (AML) and regulates cell apoptosis via the NF-kB signaling pathway. Oncotarget 8, 89643-89654, doi:10.18632/oncotarget.20288 (2017).
25 Tabe, Y. et al. Novel role of HDAC inhibitors in AML1/ETO AML cells: activation of apoptosis and phagocytosis through induction of annexin A1. Cell Death Differ 14, 1443-1456, doi:10.1038/sj.cdd.4402139 (2007).
26 Watek, M. et al. Hypogelsolinemia in Patients Diagnosed with Acute Myeloid Leukemia at Initial Stage of Sepsis. Med Sci Monit 25, 1452-1458, doi:10.12659/MSM.911904 (2019).
27 Kuzmichev, A., Nishioka, K., Erdjument-Bromage, H., Tempst, P. & Reinberg, D. Histone methyltransferase activity associated with a human multiprotein complex containing the Enhancer of Zeste protein. Genes Dev 16, 2893-2905, doi:10.1101/gad.1035902 (2002).
28 Allen, H. F., Wade, P. A. & Kutateladze, T. G. The NuRD architecture. Cell Mol Life Sci 70, 3513-3524, doi:10.1007/s00018-012-1256-2 (2013).
29 Casas, S. et al. Changes in apoptosis-related pathways in acute myelocytic leukemia. Cancer Genet Cytogenet 146, 89-101, doi:10.1016/s0165-4608(03)00102-x (2003).
30 Morito, D. et al. Gp78 cooperates with RMA1 in endoplasmic reticulum-associated degradation of CFTRDeltaF508. Mol Biol Cell 19, 1328-1336, doi:10.1091/mbc.E07-06-0601 (2008).
31 Xu, C. et al. Binding of different histone marks differentially regulates the activity and specificity of polycomb repressive complex 2 (PRC2). Proc Natl Acad Sci USA 107, 19266-19271, doi:10.1073/pnas.1008937107 (2010).
32 Liu, Z. et al. Structural basis of plant homeodomain finger 6 (PHF6) recognition by the retinoblastoma binding protein 4 (RBBP4) component of the nucleosome remodeling and deacetylase (NuRD) complex. J Biol Chem 290, 6630-6638, doi:10.1074/jbc.M114.610196 (2015).
33 Duque-Afonso, J. et al. The HDAC class I-specific inhibitor entinostat (MS-275) effectively relieves epigenetic silencing of the LAT2 gene mediated by AML1/ETO. Oncogene 30, 3062-3072, doi:10.1038/onc.2011.32 (2011).
34 Clarke, K. et al. The histone deacetylase inhibitor Romidepsin induces as a cascade of differential gene expression and altered histone H3K9 marks in myeloid leukaemia cells. Oncotarget 10, 3462-3471, doi:10.18632/oncotarget.26877 (2019).
35 Argnani, L., Broccoli, A. & Zinzani, P. L. Cutaneous T-cell lymphomas: Focusing on novel agents in relapsed and refractory disease. Cancer Treat Rev 61, 61-69, doi:10.1016/j.ctrv.2017.10.007 (2017).
36 Byrd, J. C. et al. A phase 1 and pharmacodynamic study of depsipeptide (FK228) in chronic lymphocytic leukemia and acute myeloid leukemia. Blood 105, 959-967, doi:10.1182/blood-2004-05-1693 (2005).
37 Lee, J. S. et al. Harnessing synthetic lethality to predict the response to cancer treatment. Nat Commun 9, 2546, doi:10.1038/s41467-018-04647-1 (2018).
38 Li, Y. et al. Gut microbiota dependent anti-tumor immunity restricts melanoma growth in Rnf5(−/−) mice. Nat Commun 10, 1492, doi:10.1038/s41467-019-09525-y (2019).
39 Foot, N., Henshall, T. & Kumar, S. Ubiquitination and the Regulation of Membrane Proteins. Physiol Rev 97, 253-281, doi:10.1152/physrev.00012.2016 (2017).
40 Zhao, Q. et al. GPS-SUMO: a tool for the prediction of sumoylation sites and SUMO-interaction motifs. Nucleic Acids Res 42, W325-330, doi:10.1093/nar/gku383 (2014).
41 Pabst, C. et al. Identification of small molecules that support human leukemia stem cell activity ex vivo. Nat Methods 11, 436-442, doi:10.1038/nmeth.2847 (2014).
42 Mellacheruvu, D. et al. The CRAPome: a contaminant repository for affinity purification-mass spectrometry data. Nat Methods 10, 730-736, doi:10.1038/nmeth.2557 (2013).
43 Braten, O. et al. Numerous proteins with unique characteristics are degraded by the 26S proteasome following monoubiquitination. Proc Natl Acad Sci USA 113, E4639-4647, doi:10.1073/pnas.1608644113 (2016).
44 Cox, J. & Mann, M. MaxQuant enables high peptide identification rates, individualized p.p.b.-range mass accuracies and proteome-wide protein quantification. Nat Biotechnol 26, 1367-1372, doi:10.1038/nbt.1511 (2008).
45 Shannon, P. et al. Cytoscape: a software environment for integrated models of biomolecular interaction networks. Genome Res 13, 2498-2504, doi:10.1101/gr.1239303 (2003).
46 Tzelepis, K. et al. A CRISPR Dropout Screen Identifies Genetic Vulnerabilities and Therapeutic Targets in Acute Myeloid Leukemia. Cell Rep 17, 1193-1205, doi:10.1016/j.celrep.2016.09.079 (2016).
47 Dobin, A. et al. STAR: ultrafast universal RNA-seq aligner. Bioinformaics 29, 15-21, doi:10.1093/bioinformatics/bts635 (2013).
48 Liao, Y., Smyth, G. K. & Shi, W. featureCounts: an efficient general purpose program for assigning sequence reads to genomic features. Bioinformatics 30, 923-930, doi:10.1093/bioinformatics/btt656 (2014).
49 Robinson, M. D. & Oshlack, A. A scaling normalization method for differential expression analysis of RNA-seq data. Genome Biol 11, R25, doi:10.1186/gb-2010-11-3-r25 (2010).
50 Varet, H., Brillet-Gueguen, L., Coppee, J. Y. & Dillies, M. A. SARTools: A DESeq2- and EdgeR-Based R Pipeline for Comprehensive Differential Analysis of RNA-Seq Data. PLoS One 11, e0157022, doi:10.1371/journal.pone.0157022 (2016).
51 Subramanian, A. et al. A Next Generation Connectivity Map: L1000 Platform and the First 1,000,000 Profiles. Cell 171, 1437-1452 e1417, doi:10.1016/j.cell.2017.10.049 (2017).

References 1-51 listed above are all incorporated herein by reference in their entirety for all purposes.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of treating acute myeloid leukemia (AML) in a subject in need thereof, comprising administering to the subject a therapeutically effective amount of a pharmaceutical composition comprising a really interesting new gene (RING) finger protein 5 (RNF5) inhibitor, or a retinoblastoma binding protein 4 (RBBP4) inhibitor, or both.

2. The method of claim 1, wherein the RNF5 inhibitor or the RBBP4 inhibitor comprises a short hairpin ribonucleic acid (RNA), a single guide RNA (sgRNA), or a small molecule.

3. The method of claim 1, wherein the RBBP4 inhibitor and the RNF5 inhibitor are in different pharmaceutical compositions.

4. The method of claim 1, wherein the RBBP4 and the RNF5 inhibitor are administered at different times.

5. The method of claim 1, wherein the pharmaceutical composition further comprises a histone deacetylase (HDAC) inhibitor.

6. The method of claim 5, wherein the HDAC inhibitor is selected from the group consisting of TMP269, pimelic diphenylamide 10⁶, mocetinostat, romidepsin, and N-acetyldinaline (CI-994).

7. The method of claim 1, wherein the pharmaceutical composition further comprises a compound that increases endoplasmic reticulum (ER) stress.

8. The method of claim 7, wherein the compound is thapsigargin or tunicamycin.

9. The method of claim 1, wherein the pharmaceutical composition comprises an inhibitor of endoplasmic reticulum associated protein degradation (ERAD).

10. The method of claim 9, wherein the inhibitor of ERAD comprises Eeyarestatin I.

11. The method of claim 1, wherein the pharmaceutical composition further comprises an inhibitor of unfolded protein response (UPR).

12. The method of claim 11, wherein the inhibitor of UPR comprises GSK2606414.

13. The method of claim 1, wherein the pharmaceutical composition further comprises a proteasomal inhibitor.

14. The method of claim 13, wherein the proteasomal inhibitor comprises bortezomib.

15. The method of claim 1, further comprising measuring a biomarker in a biological sample obtained from the subject prior to administering to the individual the therapeutically effective amount of the pharmaceutical composition, wherein the measuring the biomarker comprises assaying mRNA expression level and/or protein level of RNF5, RBBP4, or ubiquitinated RBBP4.

16. A method of treating acute myeloid leukemia (AML) in a subject in need thereof, comprising:
1) assaying an expression level or an amount of a biomarker in a biological sample obtained from the subject;
2) administering to the subject a therapeutically effective amount of a first pharmaceutical composition when the expression level or the amount of the biomarker is higher than a first predetermined value; and
3) administering to the subject a therapeutically effective amount of a second pharmaceutical composition when the expression level or the amount of the biomarker is lower than a second predetermined value; wherein the second pharmaceutical composition is different from the first pharmaceutical composition.

17. The method of claim 16, wherein the biomarker comprises RNF5, RBBP4, or ubiquitinated RBBP4.

18. The method of claim 16, wherein the first pharmaceutical composition comprises a RNF5 inhibitor, a RBBP4 inhibitor, a HDAC inhibitor, a UPR inhibitor, a proteasomal inhibitor, an ERAD inhibitor, or any combination thereof.

19. The method of claim 16, wherein the first predetermined value is a threshold on an average value in a cohort of AML patients.

20. The method of claim 16, wherein the therapeutically effective amount of the first pharmaceutical composition is proportional to the expression level or the amount of the biomarker measured in the subject.


SEQUENCE LISTING (40 sequences)

SEQ ID NO: 1 - DNA, 20 bp, synthetic construct
  source 1..20: mol_type = other DNA
aaagctgggatcagcagaga

SEQ ID NO: 2 - DNA, 20 bp, synthetic construct
  source 1..20: mol_type = other DNA
atcaccaaatggctggaatc

SEQ ID NO: 3 - DNA, 25 bp, synthetic construct
  source 1..25: mol_type = other DNA
atacagatgccagggctttgtatga

SEQ ID NO: 4 - DNA, 25 bp, synthetic construct
  source 1..25: mol_type = other DNA
tgggatgtctagtttccaccacaca

SEQ ID NO: 5 - DNA, 19 bp, synthetic construct
  source 1..19: mol_type = other DNA
aagcagactgcccgcaaat

SEQ ID NO: 6 - DNA, 21 bp, synthetic construct
  source 1..21: mol_type = other DNA
ggcctgtaacgatgaggtttc

SEQ ID NO: 7 - DNA, 20 bp, synthetic construct
  source 1..20: mol_type = other DNA
gctggcaggctctggggaag

SEQ ID NO: 8 - DNA, 20 bp, synthetic construct
  source 1..20: mol_type = other DNA
tgctgagtccgcagcaggtg

SEQ ID NO: 9 - DNA, 21 bp, synthetic construct
  source 1..21: mol_type = other DNA
ggaaacagagtggtcattccc

SEQ ID NO: 10 - DNA, 21 bp, synthetic construct
  source 1..21: mol_type = other DNA
ctgcttgagccgttcattctc

SEQ ID NO: 11 - DNA, 20 bp, synthetic construct
  source 1..20: mol_type = other DNA
cctctgcgctggaatcagtc

SEQ ID NO: 12 - DNA, 22 bp, synthetic construct
  source 1..22: mol_type = other DNA
ttctttctcgtcgcctcttttt

SEQ ID NO: 13 - DNA, 21 bp, synthetic construct
  source 1..21: mol_type = other DNA
caagggactggttatggtggc

SEQ ID NO: 14 - DNA, 20 bp, synthetic construct
  source 1..20: mol_type = other DNA
ccccgtcaccgataaaggtc

SEQ ID NO: 15 - DNA, 19 bp, synthetic construct
  source 1..19: mol_type = other DNA
agtccagtccatgacgcag

SEQ ID NO: 16 - DNA, 21 bp, synthetic construct
  source 1..21: mol_type = other DNA
atcaggcacgttgacatcagc

SEQ ID NO: 17 - DNA, 19 bp, synthetic construct
  source 1..19: mol_type = other DNA
tgtccgtcagaacccatgc

SEQ ID NO: 18 - DNA, 21 bp, synthetic construct
  source 1..21: mol_type = other DNA
aaagtcgaagttccatcgctc

SEQ ID NO: 19 - DNA, 21 bp, synthetic construct
  source 1..21: mol_type = other DNA
ctgcacctgacgcccttcacc

SEQ ID NO: 20 - DNA, 25 bp, synthetic construct
  source 1..25: mol_type = other DNA
cacatgaccccaccgaactcaaaga

SEQ ID NO: 21 - DNA, 21 bp, synthetic construct
  source 1..21: mol_type = other DNA
ggggcgatcaatccagagaac

SEQ ID NO: 22 - DNA, 21 bp, synthetic construct
  source 1..21: mol_type = other DNA
gtactcggtaagtgtgccctg

SEQ ID NO: 23 - DNA, 24 bp, synthetic construct
  source 1..24: mol_type = other DNA
acttttggtacattgtggcttcaa

SEQ ID NO: 24 - DNA, 20 bp, synthetic construct
  source 1..20: mol_type = other DNA
ccgccaggacaaaccagtat

SEQ ID NO: 25 - DNA, 20 bp, synthetic construct
  source 1..20: mol_type = other DNA
gcacctgtaccccggcggaa

SEQ ID NO: 26 - DNA, 20 bp, synthetic construct
  source 1..20: mol_type = other DNA
ttccgccggggtacaggtgc

SEQ ID NO: 27 - DNA, 20 bp, synthetic construct
  source 1..20: mol_type = other DNA
gttccgccggggtacaggtg

SEQ ID NO: 28 - DNA, 20 bp, synthetic construct
  source 1..20: mol_type = other DNA
cacctgtaccccggcggaac

SEQ ID NO: 29 - DNA, 25 bp, synthetic construct
  source 1..25: mol_type = other DNA
tcactttgtttttggacatagctga

SEQ ID NO: 30 - DNA, 24 bp, synthetic construct
  source 1..24: mol_type = other DNA
ccacacctagcaaccagaagttag

SEQ ID NO: 31 - DNA, 20 bp, synthetic construct
  source 1..20: mol_type = other DNA
tcatgcctgtaatcccaaca

SEQ ID NO: 32 - DNA, 20 bp, synthetic construct
  source 1..20: mol_type = other DNA
ctctgccttccaggttcaag

SEQ ID NO: 33 - DNA, 20 bp, synthetic construct
  source 1..20: mol_type = other DNA
ggtgtctaggtgctccaggt

SEQ ID NO: 34 - DNA, 20 bp, synthetic construct
  source 1..20: mol_type = other DNA
gcactctccaggaggacaca

SEQ ID NO: 35 - DNA, 21 bp, synthetic construct
  source 1..21: mol_type = other DNA
gagtgtccagtatgtaaagct

SEQ ID NO: 36 - DNA, 21 bp, synthetic construct
  source 1..21: mol_type = other DNA
cggcaagagtgtccagtatgt

SEQ ID NO: 37 - DNA, 19 bp, synthetic construct
  source 1..19: mol_type = other DNA
gaggatggattgagagaat

SEQ ID NO: 38 - DNA, 21 bp, synthetic construct
  source 1..21: mol_type = other DNA
gcctttctttcaatccttata

SEQ ID NO: 39 - DNA, 21 bp, synthetic construct
  source 1..21: mol_type = other DNA
tggtcatactgccaagatatc

SEQ ID NO: 40 - DNA, 21 bp, synthetic construct
  source 1..21: mol_type = other DNA
atgcgtcacactacgacagtg